(19) United States
(12) Patent Application Publication
(10) Pub. No.: US 2025/0269041 A1
(43) Pub. Date: Aug. 28, 2025

(54) Mutual Prodrugs of Cromoglicic Acid

(71) Applicant: Samdolite Pharmaceuticals, Inc., Saint Charles, MO (US)

(72) Inventor: Andrey Samsonov, Saint Charles, MO (US)

(73) Assignee: Samdolite Pharmaceuticals, Inc., Saint Charles, MO (US)

(21) Appl. No.: 18/858,309

(22) PCT Filed: May 16, 2023
(86) PCT No.: PCT/US2023/067055

§ 371 Date: Oct. 19, 2024

Related U.S. Application Data
(60) Provisional application No. 63/347,209, filed on May 31, 2022.
(60) Provisional application No. 63/345,514, filed on May 25, 2022.

(51) Int. Cl. A61K 47/55; A61K 47/54

(52) U.S. Cl. CPC A61K 47/55; A61K 47/542; A61K 47/545; A61K 47/551; A61K 47/552

(57) ABSTRACT

Disclosed herein are mutual prodrugs of cromoglicic acid and topical antiseptics, antibiotics, anti-inflammatory agents, keratin softeners, vasoconstrictors, retinoids and retinoid-like drugs and their compositions thereof, and methods for, e.g., treating disorders, and conditions by administration of the compositions. Topical and oral compositions of mutual prodrugs of cromoglicic acid, and derivatives and analogs of cromoglicic acid are also provided.

This application claims the benefit of priority of U.S. Provisional Patent Application Ser. No. 63/345,514 filed May 25, 2022, and also claims the benefit of priority of the U.S. Provisional Patent Application Ser. No. 63/347,209 filed May 31, 2022, the disclosures of which are each incorporated by reference in their entireties for all purposes.

The present disclosure relates to mutual prodrugs of cromoglicic acid.

According to the US National Institutes of Health (NIH), more than 15 million people in the US have symptoms of atopic dermatitis (AD), with an estimated 20% of those being infants and young children. About 60% of these infants continue to have one or more AD symptoms in adulthood. The NIH annually documents over 3 million US cases of chronic urticaria (CU) and chronic idiopathic urticaria (CIU).

Currently, topical corticosteroids are the primary anti-inflammatory treatment for AD and CU. Steroids control acute exacerbations and are used for long-term maintenance when emollients alone do not provide adequate control. However, local side effects are common, particularly skin thinning, telangiectasis, bruising, hypopigmentation, acne, striae, and secondary infection. Recently, topical formulations of the immunosuppressive drugs tacrolimus and pimecrolimus have been introduced as anti-inflammatory agents. However, there is limited evidence of their long-term safety, and other currently approved drugs (mostly antihistamines and hormones) only manage the symptoms of AD and CU without addressing a potential root cause of the disease.

Mast cells (MC) help the immune system defend tissues from diseases. MC actively participate in the innate immune response to skin barrier dysfunction and allergen penetration into deep layers of skin. During the mast cell degranulation process, they release histamines, prostaglandin 2 (PGD2), cytokine IL-17, and other substances that induce the recruitment of other innate lymphoid cells (ILC2, basophils, eosinophils) to the damaged site. Many studies suggest that mast cells are important in initiating and maintaining skin and mucous membrane inflammation in such diseases as mastocytosis, atopic dermatitis, chronic urticaria, rosacea, prurigo nodularis, acne vulgaris, inflammatory bowel disease and ulcerative colitis.

Cromoglicate sodium (the water-soluble salt of cromoglicic acid, also known as cromolyn sodium, or CS) is currently being used as a mast cell stabilizer for diseases characterized by hyperactive mast cells. However, the specialized outermost layer of skin, the stratum corneum (SC), protects against the penetration of polar and charged chemicals and microorganisms from the environment. Numerous attempts to develop topical formulations with cromolyn sodium to treat atopic dermatitis, chronic urticaria, or eczema have failed because of low transdermal absorption. One example of failure is Altoderm™, which was designed to enhance the absorption of cromolyn sodium to treat atopic dermatitis or eczema.

Despite the significant attention focused on cromoglicic acid and cromolyn sodium as topical and oral therapeutics, there is a need for compounds and methods to treat inflammatory diseases such as atopic dermatitis, chronic urticaria, rosacea, prurigo nodularis, acne vulgaris, inflammatory bowel disease, and ulcerative colitis. The present disclosure fulfills these and other needs, as evident in reference to the following disclosure.

### SUMMARY

Provided herein is a compound of Formula A:

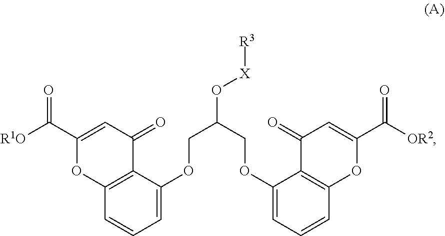

or a salt thereof, wherein:
X is chosen from a bond, —C(O), and -L-C(O)—;
L is chosen from a bond and a linker;
R¹, R², and R³ are independently chosen from hydrogen or alkyl, cycloalkyl, heterocycloalkyl, alkenyl, acetoxy, aryl, heteroaryl,

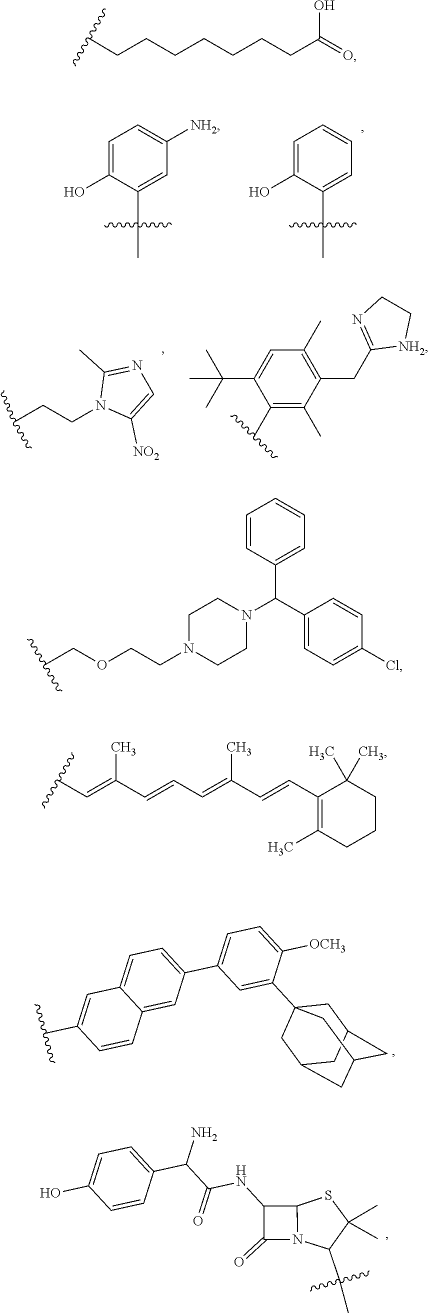

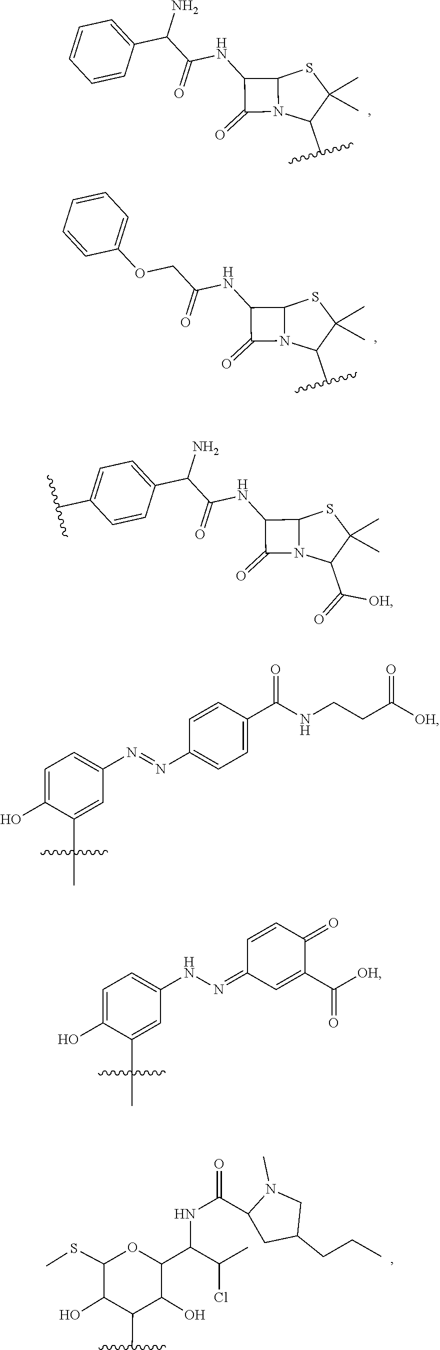

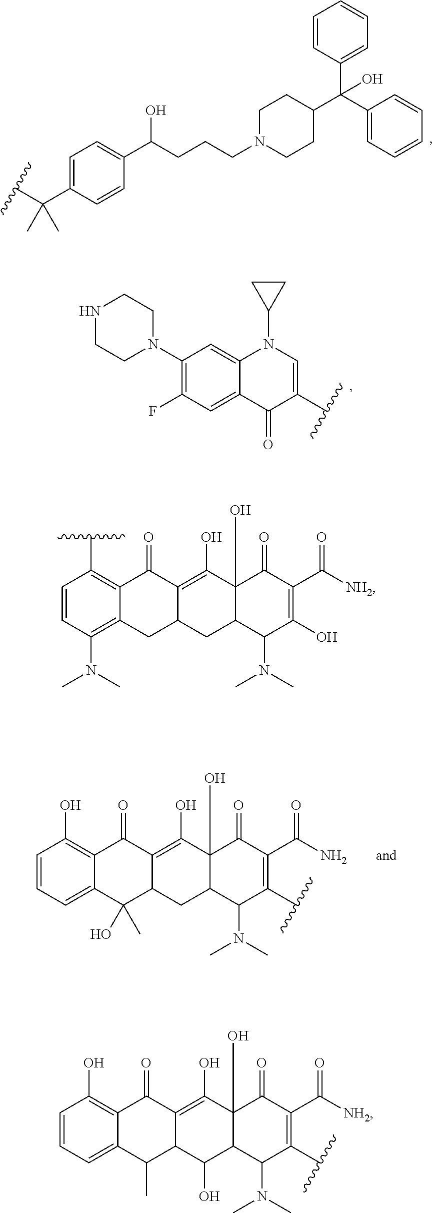 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]

any of which may be optionally substituted by one or more R⁴,
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl; and
wherein at least one of R¹, R², and R³ is chosen from

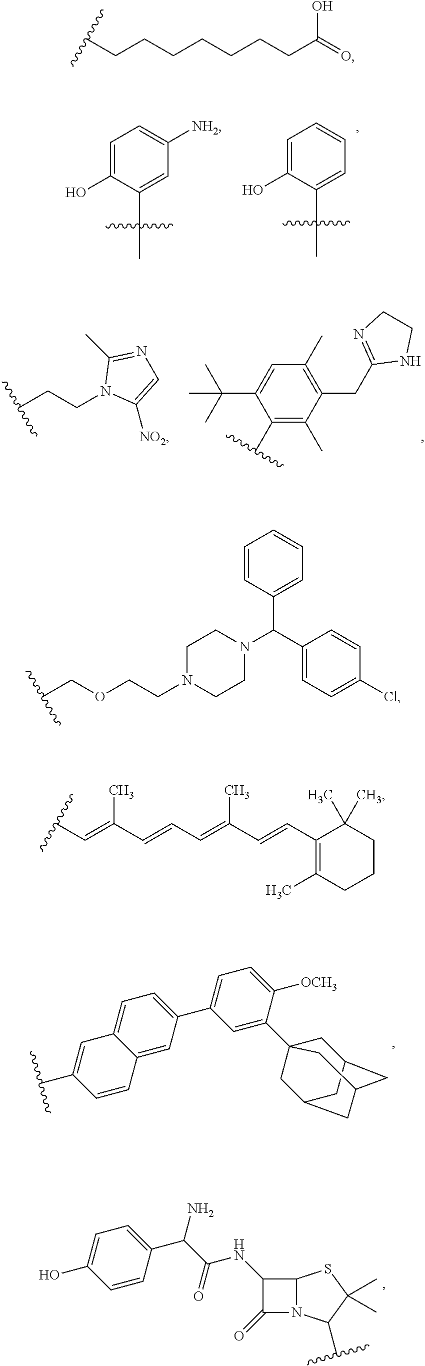

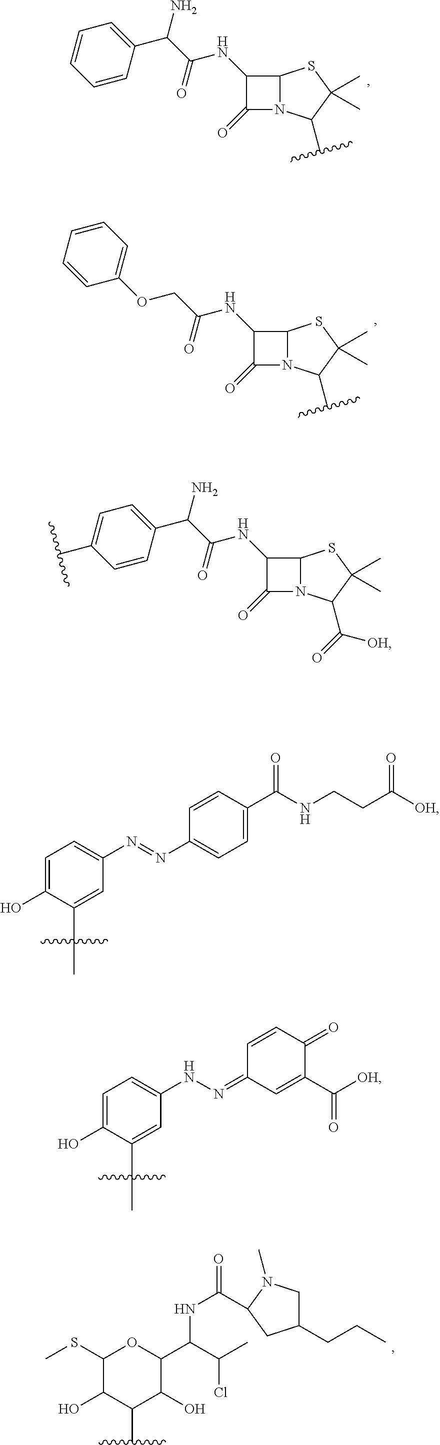

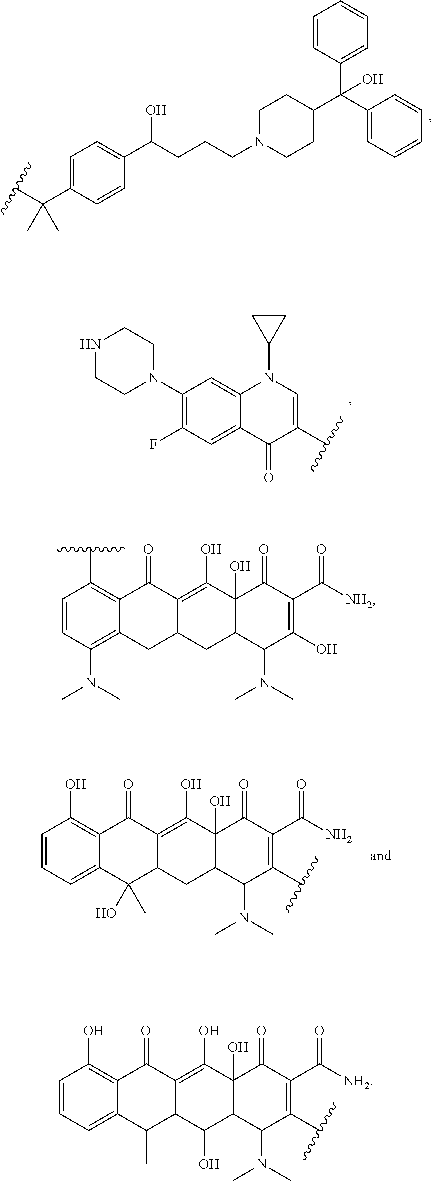 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]

Also provided is a compound disclosed herein or a salt thereof.

Also provided is a pharmaceutical formulation comprising a compound as disclosed herein, or a salt thereof, together with a pharmaceutically acceptable carrier.

Also provided is a method of inhibiting the release of chemical mediators from sensitized mast cells, comprising contacting a target cell or cells with a compound as recited herein, or a salt thereof, or a pharmaceutical composition as recited herein.

Also provided is a method of inhibition of a histaminic receptor, comprising contacting a target cell or cells with a compound as recited herein, or a salt thereof, or a pharmaceutical composition as recited herein.

Also provided is a method of treatment of a skin condition, comprising administering a therapeutically effective amount of a compound as recited herein, or a salt thereof, or a pharmaceutical composition as recited herein, to a patient in need thereof.

Also provided is method of treatment of an inflammatory disease chosen from mastocytosis, inflammatory bowel disease (IBD), Crohn's disease, and ulcerative colitis (UC), comprising administering a therapeutically effective amount of a compound as recited herein, or a salt thereof, or a pharmaceutical composition as recited herein, to a patient in need thereof.

These and other aspects of the disclosure will be apparent in the following detailed description.

### BRIEF DESCRIPTION OF THE DRAWINGS

FIG. 1 depicts a vertical Franz cell with a modified permeability membrane.

### DETAILED DESCRIPTION

In the following description, certain specific details are set forth to provide a thorough understanding of various embodiments. However, one skilled in the art will understand that the disclosure may be practiced without these details. In other instances, well-known structures have not been shown or described in detail to avoid unnecessarily obscuring descriptions of the embodiments.

As disclosed herein, in certain embodiments, cromoglicic acid is covalently linked via ester bonds with topical antiseptics, antibiotics, anti-inflammatory agents, keratin softener, vasoconstrictors, retinoids, and retinoid-like drugs to compose mutual prodrugs. The disclosure also relates to methods of preparing mutual prodrugs and their pharmaceutical compositions for preventing or alleviating the symptoms of inflammation for inflammatory diseases such as atopic dermatitis, chronic urticaria, rosacea, prurigo nodularis, acne vulgaris, inflammatory bowel disease, and ulcerative colitis. The primary target of cromolyn, the sodium salt of cromoglicic acid, is hyperactive mast cells. Other parental active pharmaceutical ingredients of mutual prodrugs disclosed herein are, for example, topical antiseptics, topical and oral antibiotics, anti-inflammatory agents, keratin softeners, vasoconstrictors, retinoids, and retinoid-like drugs.

In certain embodiments, the disclosed compounds and compositions treat chronic idiopathic urticaria. Chronic idiopathic urticaria, which has no discernable external cause, comprises most cases of chronic urticaria. Over half of all cases of CIU are thought to occur by an autoimmune mechanism. AD and CIU are chronic (long-lasting) diseases that affect the skin. They are not contagious and cannot be passed from one person to another. In atopic dermatitis, the skin becomes extremely itchy. Scratching leads to redness, swelling, cracking, “weeping” clear fluid, and crusting and scaling. The most common symptoms are dry, itchy skin and rashes on the face, inside the elbows and behind the knees, and on the hands and feet.

In certain embodiments, the disclosed compounds and compositions treat rosacea. Rosacea is even more common than AD and CU. Most people who get rosacea are between 30 and 50 years of age. Current medications for rosacea include topical antiseptics and topical antibiotics, including azelaic acid (Azelex®, Finacea®) and metronidazole (Metrogel®, Noritate®, and others). In addition, topical decongestants and vasoconstrictors like brimonidine (Mirvaso®) and oxymetazoline reduce flushing by constricting blood vessels.

In certain embodiments, the disclosed compounds and compositions treat acne vulgaris. Acne vulgaris is a chronic inflammatory skin disease that appears concomitantly with hormonal changes at puberty and constitutes the most common cutaneous disorder in adolescents and young adults. It affects the skin's pilosebaceous units, which consist of the hair shaft and the hair follicle with an attached sebaceous gland. The most common topical prescription medications for acne are retinoids and retinoid-like drugs, such as tretinoin (Avita®, Retin-A®) and adapalene. Retinoids prevent the plugging of hair follicles and can reduce sebum production. Oral antibiotics are also used to treat acne, such as tetracycline, one of its analogs such as minocycline or doxycycline, or a macrolide (erythromycin, azithromycin).

The skin's immune surveillance system is complex; several resident sentinel cells (dendritic cells, macrophages, and mast cells) are involved in perceiving the alarm signals and starting the early acne response. The role of mast cells in acne is poorly understood, but these cells support the skin's immune system. Mast cells are particularly abundant in skin and mucosa and are strategically located near blood vessels and nerve endings. The pathogenesis of acne is not fully understood. Many studies have shown that activated mast cells that produce IL-17 cytokines actively participate in the early stage of acne via stimulation of CD4⁺ T cells.

In certain embodiments, the disclosed compounds and compositions treat prurigo nodularis. Prurigo nodularis (PN) is a chronic condition with highly pruritic, hyperkeratotic papules or nodules arising in chronic pruritus. Neuronal proliferation, eosinophils, mast cells, and small-fiber neuropathy play a role in the production of pruritus in PN, although the exact mechanism has not yet been established. Treatment typically relies on topical or intralesional steroids, though more severe or recalcitrant cases use phototherapy or systemic immunosuppressives. Subcutaneous mast cell numbers have shown a significant increase in prurigo nodularis lesional skin compared to normal controls. These results indicate that hyperactive mast cells, together with cutaneous nerve fibers, are actively involved in the pathogenesis of the disease.

In certain embodiments, the disclosed compounds and compositions treat inflammatory bowel disease (IBD), Crohn's disease, and ulcerative colitis (UC). IBD is commonly used to describe both Crohn's disease and ulcerative colitis. While different diseases, Crohn's disease and ulcerative colitis both involve colon inflammation. Markedly increased numbers of mast cells have been observed in the mucosa of the ileum and colon of patients with IBD, which are accompanied by great changes in the content in mast cells, including dramatically increased expression of TNFa, IL-16, and substance P. Evidence of mast cell degranulation has been found in the walls of the intestines of patients with IBD using the immunohistochemistry technique. Highly elevated histamine and tryptase levels have been detected in the mucosa of patients with IBD, strongly suggesting that mast cell degranulation is involved in the pathogenesis of IBD. Antibiotics are used for treating the primary disease process of IBD (including luminal disease and fistulizing disease for CD and colitis in the case of UC), for treating bacterial overgrowth, or for treating septic complications of IBD, such as abscesses and post-operative wound infections. Frequently prescribed antibiotics include ciprofloxacin (Cipro®) and metronidazole. Anti-inflammatory drugs are often the first step in treating inflammatory bowel disease. Anti-inflammatoir include corticosteroids and aminosalicylates, such as mesalamine (Asacol HD®, Delzicol®, and others), balsalazide (Colazal®) and olsalazine (Dipentum®). Therefore, in certain embodiments, treating IBD and UC includes a mast cell stabilizer, an antibiotic, and an anti-inflammatory drug.

All skin and intestinal diseases mentioned above have chronic autoimmune and inflammatory mechanisms. Numerous studies indicate that hyperactive mast cells are actively involved in the pathogenesis of these diseases. MC actively participates in the innate immune response to skin barrier dysfunction and allergen penetration into deep layers of skin. MC migrates into these skin layers after allergen penetration or during the active stage of skin inflammation diseases. During the mast cell degranulation, they release histamines, prostaglandin 2 (PGD2), cytokine IL-17, and other substances that recruit other innate lymphoid cells (ILC2, basophils, eosinophils) to the damaged site. Many studies suggest that mast cells initiate and maintain skin inflammation in diseases like atopic dermatitis, chronic urticaria, rosacea, prurigo nodularis, acne vulgaris, inflammatory bowel disease, and ulcerative colitis probably through induction of secretion of IL-13, TSLP, and IL-4. It has been demonstrated in an eczema animal model that inducible selective expression of IL-13 in mouse skin causes AD-like phenotypes, including increased mast cells and mediators.

In certain embodiments, the simultaneous application of mast cell stabilizers and other medications that help to alleviate inflammation and suppress bacterial infection in damaged sites has a synergetic effect. In such embodiments, mast cell stabilizers are combined with other APIs such as anti-inflammatory, antibacterial, topical antiseptics, antibiotics, retinoids, and retinoid-like drugs. For example, for intestinal inflammatory diseases such as IBD and UC, mast cell stabilizers are combined with antibiotics like ciprofloxacin (Cipro®) and metronidazole and/or with anti-inflammatoires compounds aminosalicylates, such as mesalamine (Asacol HD®, Delzicol®, and others), balsalazide (Colazal®) and olsalazine (Dipentum®).

In certain embodiments, mast cell stabilizers and other APIs are combined separately. These drugs may be chemically conjugated to enhance pharmacological activity, prevent clinical side effects, or improve pharmaceutical properties, such as stability, lipophilicity, and transdermal permeability.

Cromoglicate sodium (the water-soluble salt of cromoglicic acid, also known as cromolyn sodium or CS) is currently used as a mast cell stabilizer for diseases characterized by hyperactive mast cells. This drug prevents the release of inflammatory chemicals such as histamine and tryptase/chymase from mast cells. There is evidence that treatment with cromoglicate sodium treats the disease itself, as it has been shown to slow the progression of inflammatory diseases such as asthma. Cromoglicate sodium is thus expected to be effective in treating asthma and mastocytosis and is currently regulated in the United States as an active pharmaceutical ingredient. Cromoglicate sodium is typically administered orally but has poor bioavailability. A small percentage of the active ingredient (e.g., 1-2%) is believed to be available following oral administration. At a normal pH of 7.4, cromolyn sodium has logP=−4.8 because it contains two negatively charged carboxylic groups and one polar hydroxy group. Therefore, cromolyn sodium is unsuitable for transdermal or submucosal delivery despite being a mast cell stabilizer.

### Definitions

Unless the context requires otherwise, throughout the specification and claims which follow, the word “comprise” and variations thereof, such as “comprises” and “comprising,” are to be construed in an open, inclusive sense, that is, as “including, but not limited to.” Further, the headings provided herein are for convenience only and do not interpret the scope or meaning of the claimed disclosure.

Reference throughout this specification to “one embodiment,” “an embodiment,” “some embodiments,” or “a certain embodiment” means that a particular feature, structure, or characteristic described for the embodiment is included in at least one embodiment. Thus, the appearances of the phrases “in one embodiment,” “in an embodiment,” “in some embodiments,” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

As used herein, “about” means ±20% of the stated value and includes, more specifically, values of ±10%, ±5%, ±2%, and ±1% of the stated value.

The term “alkenyl,” as used herein, refers to a straight-chain or branched-chain hydrocarbon radical having one or more double bonds and containing from 2 to 20 carbon atoms. In some embodiments, alkenyl will comprise 2 to 6 carbon atoms. The term “alkenylene” refers to a carbon-carbon double bond system attached at two or more positions, such as ethenylene [(—CH═CH—), (—C::C—)].

The term “alkoxy” and, interchangeably, “(alkyl)oxy,” as used herein, refers to an alkyl radical attached to a molecule by oxygen.

The term “alkyl,” as used herein, refers to a straight-chain or branched-chain saturated hydrocarbon radical containing from 1 to 20 carbon atoms. In some embodiments, alkyl will comprise from 1 to 10 carbon atoms. In some embodiments, alkyl will comprise from 1 to 8 carbon atoms.

The term “alkynyl,” as used herein, refers to a straight-chain or branched chain hydrocarbon radical having one or more triple bonds and containing from 2 to 20 carbon atoms. In certain embodiments, said alkynyl comprises 2 to 6 carbon atoms. In further embodiments, said alkynyl comprises 2 to 4 carbon atoms.

The terms “amido” and “carbamoyl,” as used herein, refer to an amino group described below attached to the parent molecular moiety through a carbonyl group or vice versa. The term “C-amido,” as used herein, refers to a —C(O)N(RR′) group, wherein R and R′ are independently chosen from hydrogen, alkyl, heteroalkyl, aryl, cycloalkyl, heteroaryl, and heterocycloalkyl. Additionally, R and R′ may combine with the amino nitrogen atom to form heterocycloalkyl. The term “N-amido,” as used herein, refers to an RC(O)N(R′)— group, wherein R and R′ are independently chosen from hydrogen, alkyl, heteroalkyl, aryl, cycloalkyl, heteroaryl, and heterocycloalkyl.

The term “amino,” as used herein, refers to —NRR′, wherein R and R′ are independently chosen from hydrogen, alkyl, acyl, heteroalkyl, aryl, cycloalkyl, heteroaryl, and heterocycloalkyl. Additionally, R and R′ may combine with the amino nitrogen atom to form heterocycloalkyl.

The term “aryl,” as used herein, means a carbocyclic aromatic system containing one, two, or three rings wherein such polycyclic ring systems are fused.

The term “carbonyl,” as used herein, is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is chosen from alkyl and cycloalkyl. A “C-carboxy” group refers to a —C(O)OR groups where R is chosen from alkyl and cycloalkyl.

As used herein, the term “cycloalkyl” or “carbocycle” refers to a saturated monocyclic, bicyclic, or tricyclic alkyl group wherein each cyclic moiety contains from 3 to 12 carbon atom ring members. In some embodiments, cycloalkyl will comprise 5 to 7 carbon atoms. In some embodiments, cycloalkyl will comprise a spirocyclic ring system. “Bicyclic” and “tricyclic” as used herein are intended to include both fused ring systems, as well as the multicyclic (multicentered) saturated type.

The term “halo,” or “halogen,” as used herein, refers to fluorine, chlorine, bromine, or iodine.

The term “haloalkoxy,” as used herein, refers to a haloalkyl group attached to the parent molecular moiety through an oxygen atom.

The term “haloalkyl,” as used herein, refers to an alkyl radical with the meaning defined above wherein one or more hydrogens are replaced with a halogen. Specifically embraced are monohaloalkyl, dihaloalkyl, and polyhaloalkyl radicals. Dihalo and polyhaloalkyl radicals may have two or more of the same halo atoms or a combination of different halo radicals.

The term “heteroaryl,” as used herein, refers to a 3 to 15-membered unsaturated heteromonocyclic ring or a fused monocyclic, bicyclic, or tricyclic ring system in which at least one of the fused rings is aromatic, which contains at least one atom chosen from N, O, and S. In some embodiments, heteroaryl will comprise from 1 to 4 heteroatoms as ring members. In some embodiments, heteroaryl will comprise from 1 to 2 heteroatoms as ring members. In some embodiments, heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings.

The terms “heterocycloalkyl” and, interchangeably, “heterocycle,” as used herein, refers to a saturated, partially unsaturated, or fully unsaturated (but nonaromatic) monocyclic; saturated, partially unsaturated, or fully unsaturated (but not fully aromatic) bridged; saturated, partially unsaturated, or fully unsaturated (but not fully aromatic) bicyclic; or saturated, partially unsaturated, or fully unsaturated (but not fully aromatic)tricyclic heterocyclic group containing at least one heteroatom as a ring member wherein each heteroatom may be independently chosen from nitrogen, oxygen, and sulfur.

In some embodiments, heterocycloalkyl will comprise a spirocyclic ring system. In some embodiments, heterocycloalkyl will comprise from 1 to 4 heteroatoms as ring members. In some embodiments, heterocycloalkyl will comprise from 1 to 2 heteroatoms as ring members. In some embodiments, heterocycloalkyl will comprise from 3 to 8 ring members in each ring. In some embodiments, heterocycloalkyl will comprise from 3 to 7 ring members in each ring. In some embodiments, heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl or heteroaryl group, as defined herein, or an additional heterocycle group.

The terms “hydroxy” and, interchangeably, “hydroxyl,” as used herein, refer to —OH.

Asymmetric centers exist in the compounds and pharmaceutically acceptable salts thereof, disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds, and pharmaceutically acceptable salts thereof, can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds, and pharmaceutically acceptable salts thereof, of particular stereochemistry are either commercially available or can be made and resolved by techniques known in the art. Additionally, the compounds, and pharmaceutically acceptable salts thereof, disclosed herein may exist as geometric isomers. The present disclosure includes all cis, trans, syn, anti, entgegen (E), and zusammen (Z) isomers and the appropriate mixtures thereof.

Additionally, the compounds disclosed herein can exist in unsolvated and solvated forms with pharmaceutically acceptable solvents such as water, ethanol, and the like. In general, the solvated forms are considered equivalent to the unsolvated forms.

The term “bond” refers to a covalent linkage between two atoms or moieties when the atoms joined by the bond are part of a larger substructure. A bond may be single, double, or triple unless otherwise specified. For example, a dashed line between two atoms in a drawing of a molecule indicates that an additional bond may be present or absent at that position.

As used herein, “administering to a patient” refers to introducing a composition or dosage form into the patient via an art-recognized means of introduction.

The term “disease,” as used herein, is intended to be generally synonymous. It is used interchangeably with the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means administering two or more therapeutic agents to treat a therapeutic condition or disorder described in the present disclosure. Such administration encompasses the co-administration of these therapeutic agents in a substantially simultaneous manner, such as in a single capsule having a fixed ratio of active ingredients or in multiple, separate capsules for each active ingredient. In addition, such administration also encompasses the use of each type of therapeutic agent sequentially. In either case, the treatment regimen will provide beneficial effects of the drug combination in treating the conditions or disorders described herein.

The phrase “therapeutically effective” is intended to qualify the amount of active ingredients used in treating a disease or disorder or on the effecting of a clinical endpoint. The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the clinician's judgment.

As used herein, the term “treat,” “treating,” or “treatment” means administering therapy to an individual who already manifests at least one symptom of a disease or condition or who has previously manifested at least one symptom of a disease or condition. For example, “treating” can include alleviating, abating, or ameliorating a disease or condition's symptoms, preventing additional symptoms, ameliorating the underlying metabolic causes of symptoms, inhibiting the disease or condition, e.g., arresting developing the disease or condition, relieving the disease or condition, causing regression of the disease or condition, relieving a condition caused by the disease or condition, or stopping the symptoms of the disease or condition. For example, the term “treating” a disorder means reducing the severity of one or more symptoms for that particular disorder. Therefore, treating a disorder does not necessarily mean a reduction in the severity of all symptoms of a disorder and does not necessarily mean a complete reduction in the severity of one or more symptoms of a disorder.

The term “patient” is generally synonymous with the term “subject” and includes all mammals, including humans. Examples of patients include humans, livestock such as cows, goats, sheep, pigs, and rabbits, and companion animals such as dogs, cats, rabbits, and horses. In certain embodiments, the patient is a human.

### Prodrugs

As used herein, the term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

As used herein, the term “mutual prodrug” refers to a compound comprised of two or more chemically conjugated APIs with separate pharmacological activities. In a mutual prodrug, each component drug functions as the “pro” portion with respect to the other. Like a prodrug, a mutual prodrug is converted into the component active drugs within the body through enzymatic or non-enzymatic reactions. The effect of this cleavage of the mutual prodrug is a simultaneous application of all linked APIs at the site of cleavage. In some embodiments, the linkage of two or more drugs with polar and charged groups (like carboxyl and/or hydroxy groups) increases the lipophilicity of the mutual prodrug. In some embodiments, the mutual prodrug may have a higher permeability through hydrophobic barriers, such as human skin, intestinal epithelial cell lipid membranes, or the blood-brain barrier.

The present disclosure comprises a method to convert the hydrophilic and polar mast cell stabilizer, cromolyn sodium, into a lipophilic mutual prodrug. Such a mutual prodrug is composed of two or three drug compounds covalently linked with each other via ester bonds. When covalently linked, the drug components are rendered pharmaceutically inactive, and the mutual prodrug possesses greater hydrophobicity than the individual components alone. The ester linkages between components are cleavable. For example, the linkages may be hydrolyzable and/or may be enzymatically cleavable. Preferably, the linkages are cleavable under physiological conditions such as those in a mammalian body, particularly a human body. The ester linkages are easily degraded by mammalian esterase, thereby allowing the release of each drug component in vivo. After ester bond cleavage, each drug component is rendered pharmaceutically active.

In some embodiments, the mutual prodrugs of the present disclosure exhibit aqueous/lipid solubility profiles different from those of each parental drug individually. In addition to facilitating the delivery of the active components, linking the components may impart a protective effect on the mutual prodrug, thereby reducing or preventing unwanted degradation and/or side effects of either or both of the drugs before the cleavage of the mutual prodrug in vivo. In some embodiments, the lipophilicity of the mutual prodrug is increased by linking a neutral lipophilic compound with a high log P. In some embodiments, the neutral lipophilic compound is heptanoic acid.

In some embodiments, cromoglicate sodium, or a derivative or analog thereof, is covalently linked with an anti-inflammatory agent, a topical antiseptic, a topical antibiotic, a keratin softener, a vasoconstrictor, a retinoid or a retinoid-like drug to form a mutual prodrug, as described herein. The disclosure provides pharmaceutical compositions for topical and/or transdermal applications of a mutual prodrug.

In some embodiments, the mutual prodrug is a compound having the structural formula:

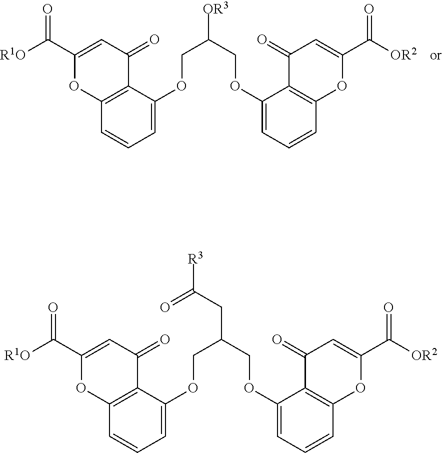

wherein R¹, R², and R³ are independently chosen from H and active pharmaceutical ingredients chosen from topical antiseptics, topical or oral antibiotics, anti-inflammatory agents, keratin softener, vasoconstrictors, retinoids, and retinoid-like drugs, wherein at least one of R¹, R², and R³ must be an active pharmaceutical ingredient. In some embodiments, the active pharmaceutical ingredients are linked via ester bonds.

In some embodiments, cromoglicate sodium, or a derivative or analog thereof, is covalently linked with one or more organic groups to form a lipophilic prodrug, as described herein. The disclosure provides pharmaceutical compositions for topical and/or transdermal applications of a lipophilic prodrug.

In some embodiments, the lipophilic prodrug is a compound having the structural formula:

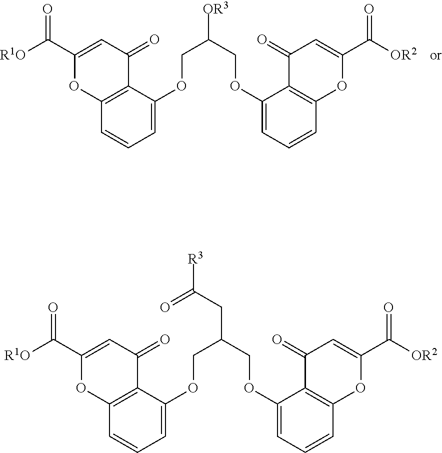

wherein R¹, R², and R³ are independently chosen from H, alkyl, acetoxymethyl, alkenyl, benzyl, and aryl, any of which may be optionally substituted by one or more hydroxyl groups. In some embodiments, the active pharmaceutical ingredients are linked via ester bonds.

In some embodiments, R¹, R², and R³ are independently chosen from H, methyl, ethyl, propyl, isopropyl, butyl, isobutyl, tert-butyl, pentyl, isopentyl, tert-pentyl, hexyl, isohexyl, heptyl, isoheptyl, phenyl, benzyl, and acetoxymethyl. In some embodiments, the phenyl is substituted with hydroxyl.

In some embodiments, hydrophilic cromolyn sodium is converted into its lipophilic mutual prodrug via the covalent linking of one, two, or three API molecules to cromoglicic acid.

In some embodiments, hydrophilic cromolyn sodium is converted into a lipophilic mutual prodrug via the covalent linking of two API molecules to two carboxylic groups of cromoglicic acid. In some embodiments, the API comprises one or more functional hydroxy group(s) that form ester bonds with functional carboxylic groups of cromoglicic acid. In some embodiments, the product of the conversion is a double mutual prodrug that, after hydrolysis by endogenous esterases, will release one molecule of cromoglicate sodium and two molecules of other APIs chosen from anti-inflammatory agents, topical antiseptics, topical antibiotics, keratin softeners, vasoconstrictors, retinoids, and retinoid-like drugs.

In some embodiments, hydrophilic cromolyn sodium is converted into a lipophilic mutual prodrug via covalently linking one API molecule to a hydroxy group of cromoglicic acid. In some embodiments, the API contains functional carboxyl group(s) that form ester bonds with the functional hydroxy group of cromoglicic acid. In some embodiments, the product of the conversion will be a double mutual prodrug that, after hydrolysis by endogenous esterases, releases one molecule of cromoglicate sodium and one molecule of another API that is chosen from an anti-inflammatory agent, a topical antiseptic, a topical antibiotic, a keratin softener, a vasoconstrictor, a retinoid, and a retinoid-like drug.

In some embodiments, hydrophilic cromolyn sodium is converted into a lipophilic mutual prodrug via covalently linking two equal API molecules to two carboxylic groups of cromoglicic acid and covalent linking of one molecule of different API to one hydroxy group of cromoglicic acid. In some embodiments, the product of the conversion will be a triple mutual prodrug that, after hydrolysis by endogenous esterases, releases one molecule of cromoglicate sodium, two molecules of APIs chosen from anti-inflammatory agents, topical antiseptics, topical antibiotics, keratin softeners, vasoconstrictors, retinoids and retinoid-like drugs, and one molecule of another API that is chosen from an anti-inflammatory agent, a topical antiseptic, a topical antibiotic, a keratin softener, a vasoconstrictor, a retinoid, and a retinoid-like drug.

In some embodiments, parental APIs are compounds already approved by FDA for treatment and alleviation of symptoms of atopic dermatitis, chronic urticaria, rosacea, prurigo nodularis, acne vulgaris, inflammatory bowel disease, and ulcerative colitis. In some embodiments, parental APIs for mutual prodrugs are selected based on the disease type. In some embodiments, parental APIs for skin inflammation diseases are chosen from anti-inflammatory agents, topical antiseptics, topical and oral antibiotics, keratin softeners, vasoconstrictors, and retinoid and retinoid-like drugs.

In some embodiments, the antibiotic is chosen from ciprofloxacin, clindamycin, minocycline, doxycycline, tetracycline, amoxicillin, ampicillin, penicillin, and metronidazole. In some embodiments, the anti-inflammatory agent is chosen from aminosalicylates, mesalamine, balsalazide, olsalazine, azelaic acid, cetirizine, and fexofenadine. In some embodiments, the vasoconstrictor is chosen from oxymetazoline. In some embodiments, the keratin softener is salicylic acid. In some embodiments, the retinoid is chosen from tretinoin and adapalene.

Mesalamine, balsalazide, and olsalazine treat and prevent mild to moderately active ulcerative colitis flare-ups. They work inside the bowels to reduce inflammation and other disease symptoms. They help reduce ulcerative colitis symptoms such as bowel inflammation, diarrhea, rectal bleeding, and stomach pain. To be a parental drug for a mutual prodrug of cromoglicic acid, the API must have at least one functional hydroxy group, at least one functional carboxyl group, or both hydroxy and carboxyl groups.

In some embodiments, the mutual prodrug uses an ester linkage formed synthetically through the reaction of functional carboxyl groups on one drug with functional hydroxy groups on another drug. Ester linkages formed between two compounds result from a Fisher esterification reaction. These ester linkages are degraded by extracellular and intracellular mammalian esterases, thereby allowing the release of each drug component in vivo. After hydrolysis by endogenous esterases, each mutual prodrug component becomes pharmaceutically active. Thus, after administration to a patient, cleavage of the mutual prodrug permits each drug component, pharmaceutically activated by cleavage, to produce its respective intended pharmacological action. In addition, in the proposed mutual prodrug of cromoglicic acid, each component facilitates the transmembrane and transdermal delivery of the other component by “masking” their charged and polar groups, such as carboxyl and hydroxy groups.

In some embodiments, the lipophilicity of the mutual prodrug of cromoglicic acid is increased via a combination of hydrophilic APIs (log P<0) attached to carboxyl groups of cromoglicic acid and one molecule of hydrophobic API (log P>2-5) attached to the hydroxy group of cromoglicic acid. This combination significantly increases the mutual prodrug log P to a feasible level for topical application.

In some embodiments, the lipophilicity of the mutual prodrug of cromoglicic acid is increased via a combination of hydrophilic APIs (log P<0) attached to the hydroxy group of cromoglicic acid and two molecules of hydrophobic APIs (log P>2-5) attached to the carboxyl groups of cromoglicic acid.

In some embodiments, the lipophilicity of mutual prodrug of cromoglicic acid is increased via a combination of hydrophilic APIs (log P<0) attached to carboxyl groups of cromoglicic acid with a long chain carboxylic acid (log P>2-5) attached to the hydroxy group of cromoglicic acid.

In some embodiments, the lipophilicity of mutual prodrug of cromoglicic acid is increased via a combination of hydrophilic APIs (log P<0) attached to the hydroxy group of cromoglicic acid with long chain alcohols (log P>2-5) attached to the carboxyl groups of cromoglicic acid.

Provided herein is a compound of Formula A:

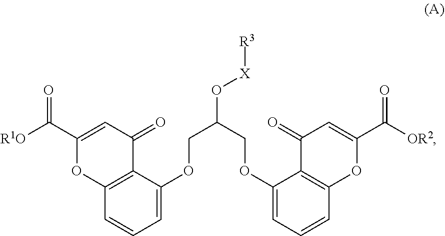

or a salt thereof, wherein:
X is chosen from a bond, —C(O), and -L-C(O)—;
L is chosen from a bond and a linker;
R¹, R², and R³ are independently chosen from hydrogen or alkyl, cycloalkyl, heterocycloalkyl, alkenyl, acetoxy, aryl, heteroaryl,

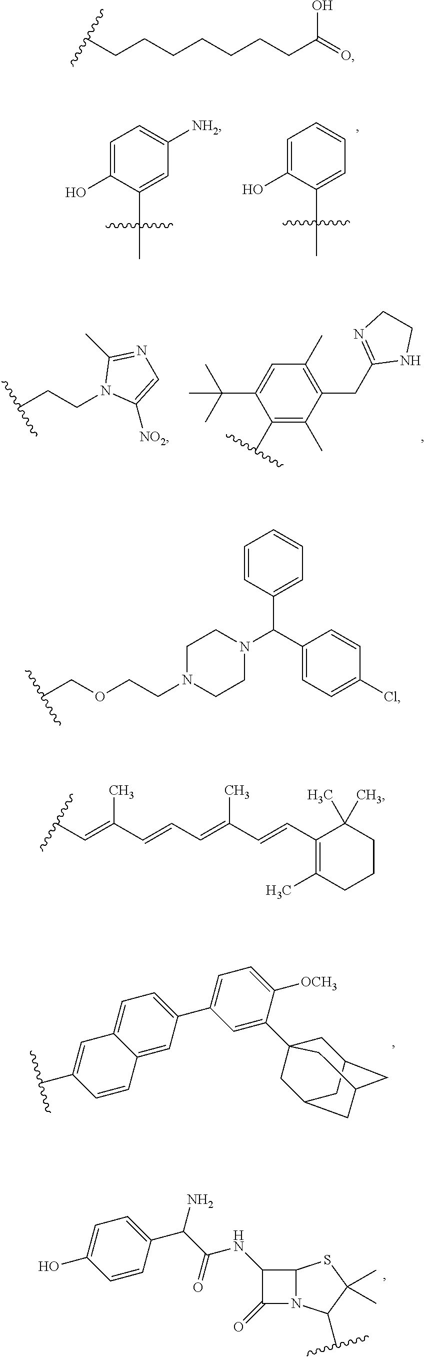

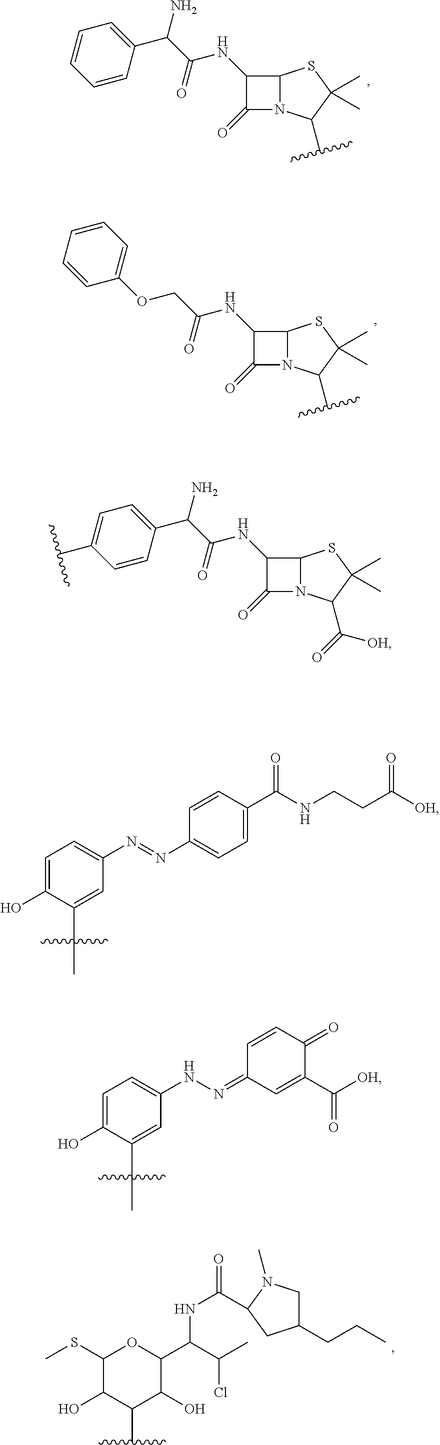

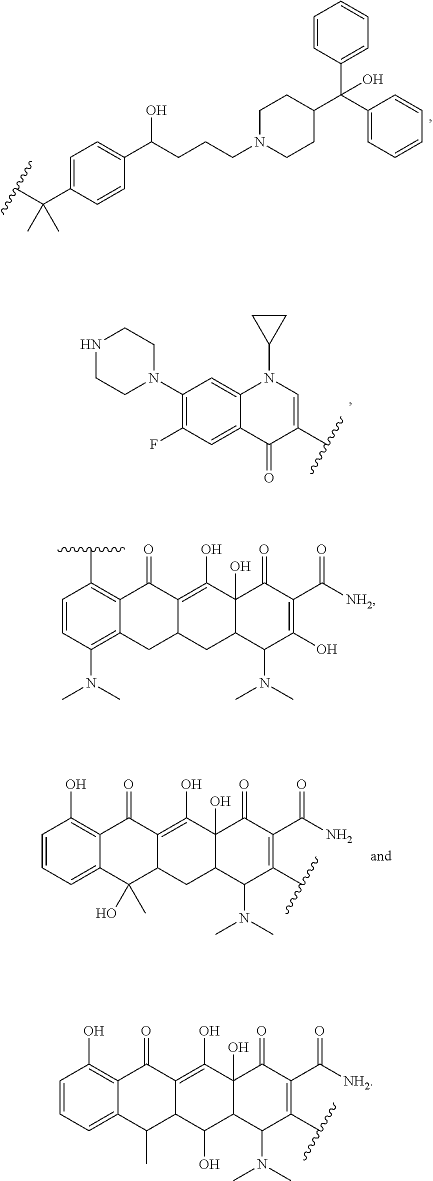 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]

any of which may be optionally substituted by one or more R⁴;
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl; and
wherein at least one of R¹, R², and R³ is chosen from

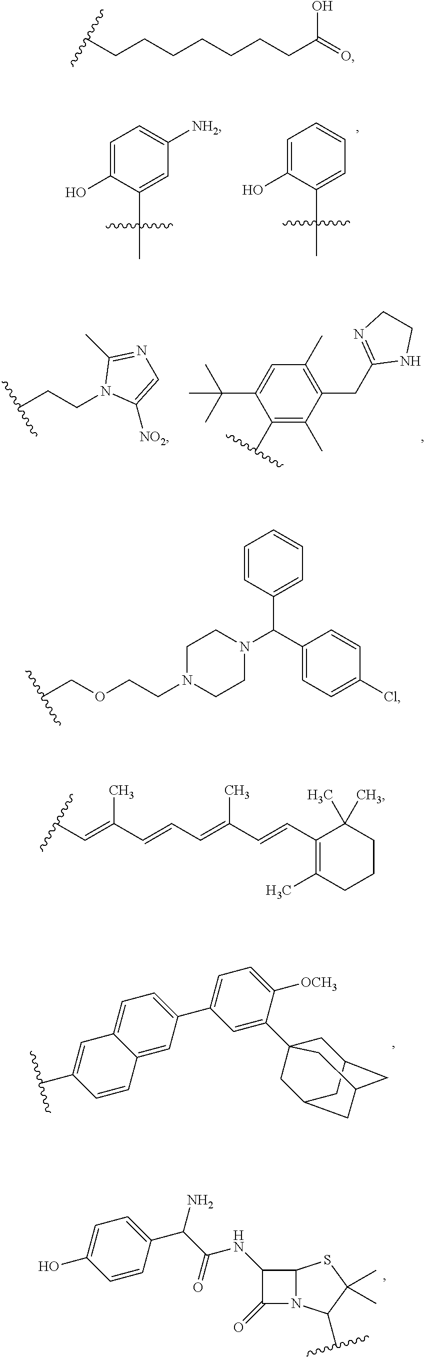

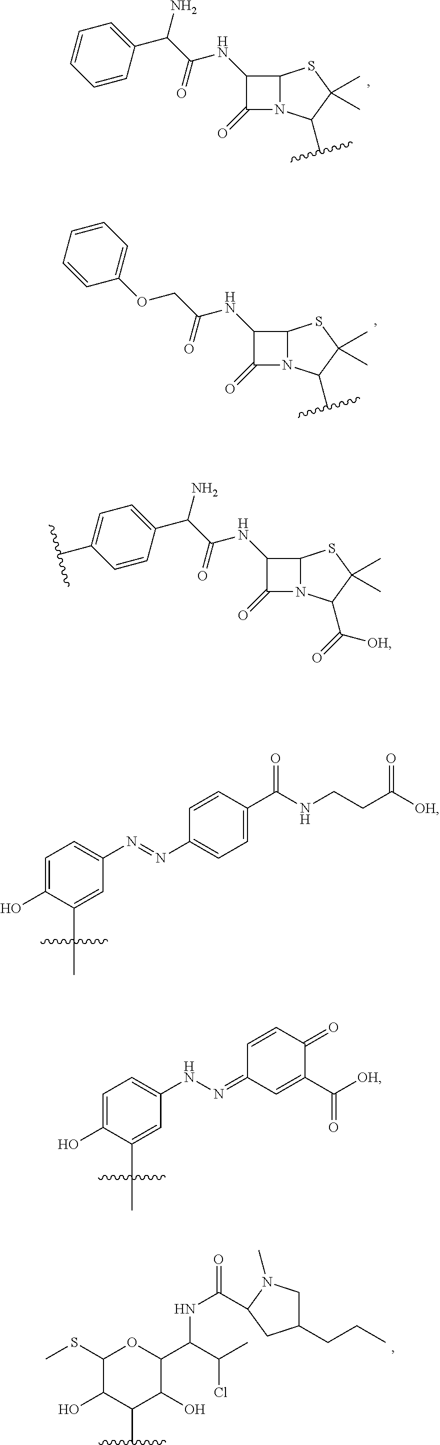

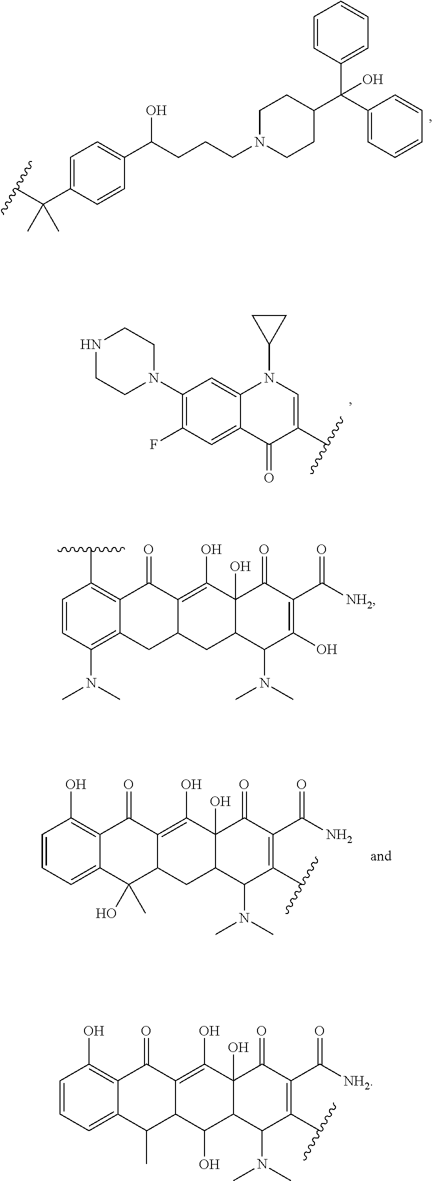 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]

In some embodiments, the compound is a compound of Formula I:

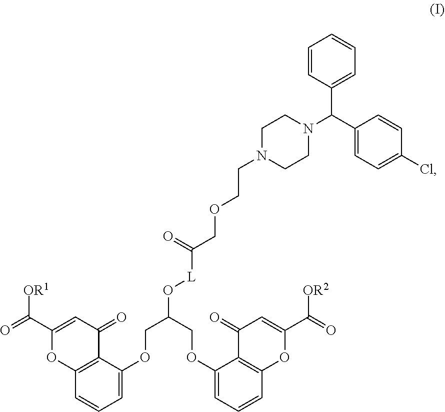

or a salt thereof, wherein:
L is chosen from a bond and a linker;
R¹ and R² are independently chosen from hydrogen or alkyl, cycloalkyl, heterocycloalkyl, alkenyl, acetoxy, aryl, heteroaryl,

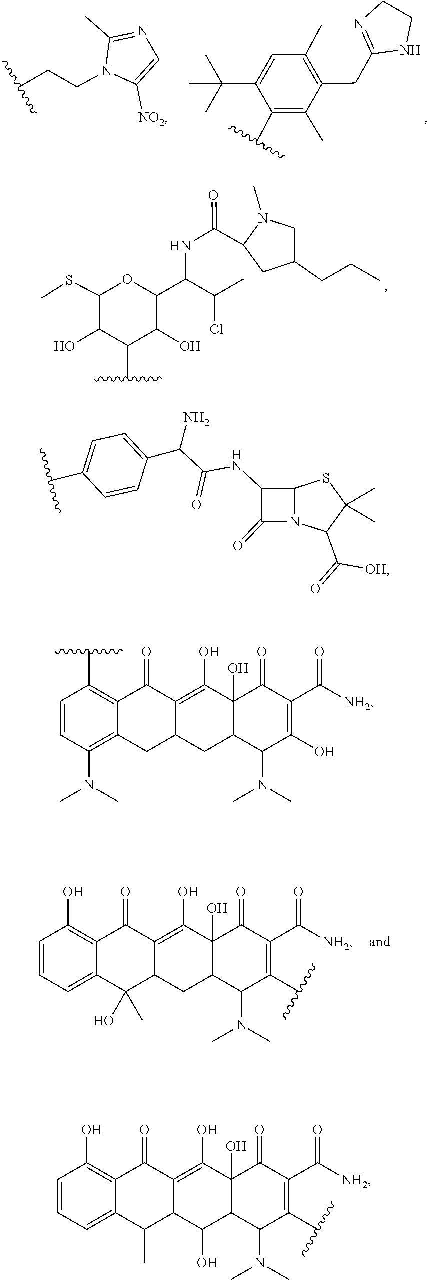

any of which may be optionally substituted by one or more R⁴; and
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carbonyl.

In some embodiments, L is a bond.

In some embodiments, L is a linker.

In some embodiments, the linker is hydrolyzable and/or enzymatically cleavable. In some embodiments, the linkage is cleavable under physiological conditions in human skin layers epidermis and dermis, such as esterases catalyzing the hydrolysis of esters, thioesters, amides, and amidases. In some embodiments, the linkage between the first component and L and between L and second component are cleavable.

In some embodiments, L is chosen from —C(O)—(CH₂)ₙ—O—, and —C(O)—(CH₂CH₂O)ₙ—, wherein n is an integer is chosen from 1 to 12.

In some embodiments, R¹ and R² are hydrogen.

In some embodiments, R¹ and R² are independently chosen from C₁-C₈ alkyl, C₃-C₈ cycloalkyl, C₃-C₈ heterocycloalkyl, C₂-C₈ alkenyl, aryl, and heteroaryl, any of which may be optionally substituted by one or more R².

In some embodiments, R¹ and R² are C₁-C₈ alkyl optionally substituted by one or more R².

In some embodiments, R¹ and R² are chosen from methyl, ethyl, n-propyl, isopropyl, n-butyl, t-butyl, and n-pentyl, any of which may be optionally substituted by one R².

In some embodiments, R¹ and R² are benzyl.

In some embodiments, R¹ and R² are chosen from

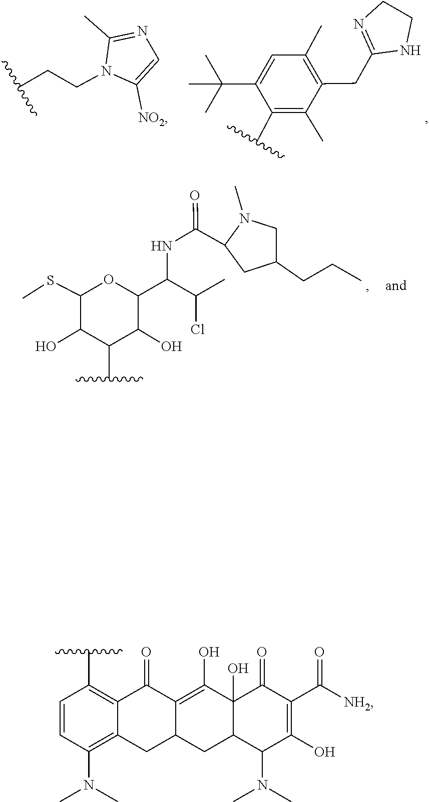

In some embodiments, the compound of Formula I is chosen from:

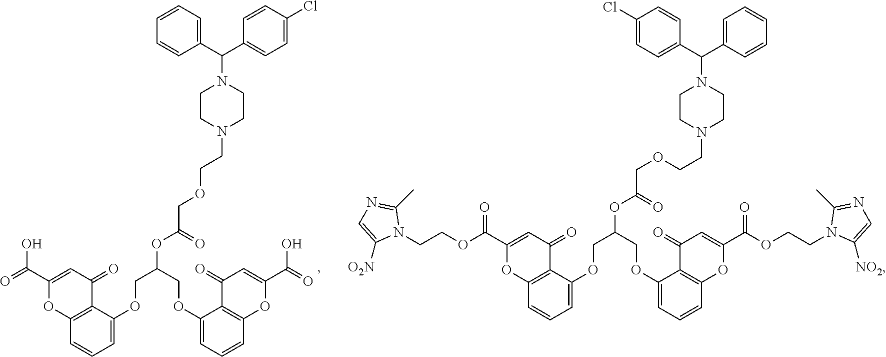 [SMILES: O=C(COCCN1CCN(C(c2ccccc2)c2ccc(Cl)cc2)CC1)OC(COc1cccc2oc(C(=O)O)cc(=O)c12)COc1cccc2oc(C(=O)O)cc(=O)c12 ; Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)COCCN3CCN(C(c4ccccc4)c4ccc(Cl)cc4)CC3)cccc2o1]

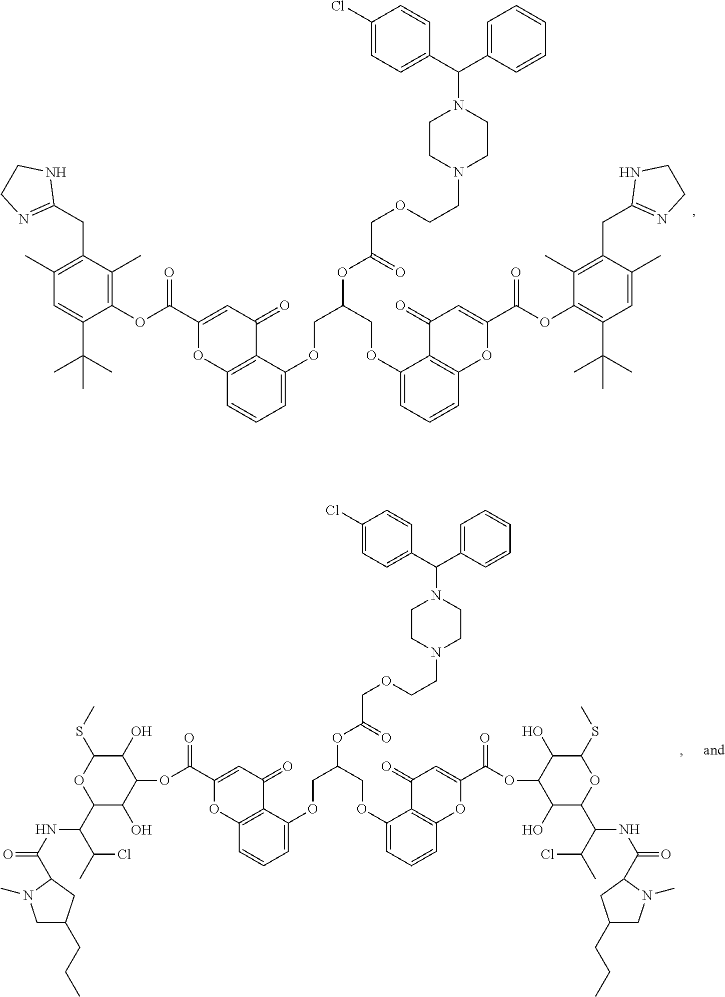

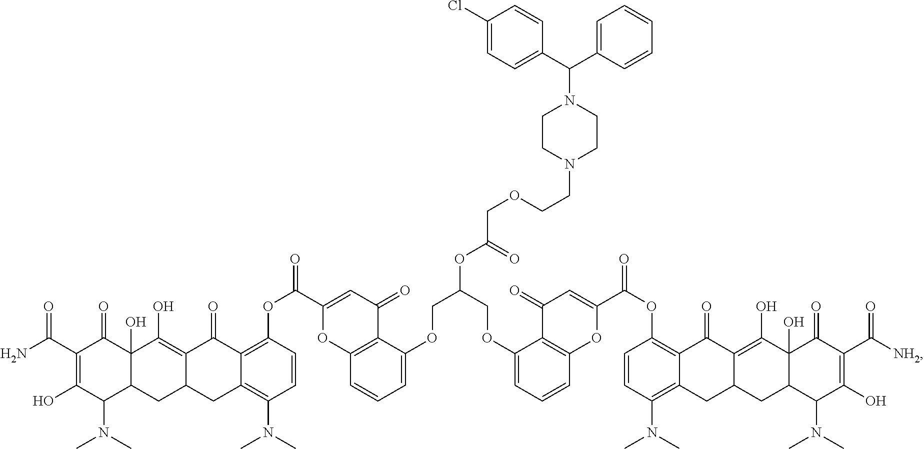 [SMILES: CN(C)c1ccc(OC(=O)c2cc(=O)c3c(OCC(COc4cccc5oc(C(=O)Oc6ccc(N(C)C)c7c6C(=O)C6=C(O)C8(O)C(=O)C(C(N)=O)=C(O)C(N(C)C)C8CC6C7)cc(=O)c45)OC(=O)COCCN4CCN(C(c5ccccc5)c5ccc(Cl)cc5)CC4)cccc3o2)c2c1CC1CC3C(N(C)C)C(O)=C(C(N)=O)C(=O)C3(O)C(O)=C1C2=O]

or a salt thereof.

In some embodiments, the compound is a compound of Formula II:

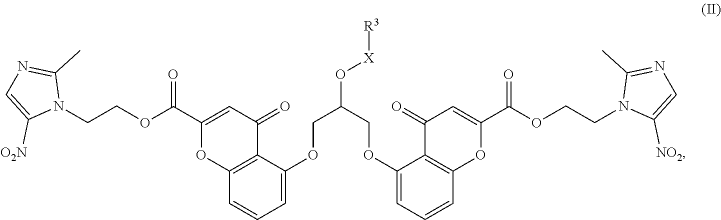

or a salt thereof, wherein:
X is —C(O)— or a bond;
R³ is hydrogen or is chosen from alkyl, cycloalkyl, heterocycloalkyl, alkenyl, alkoxy, aryl, heteroaryl,

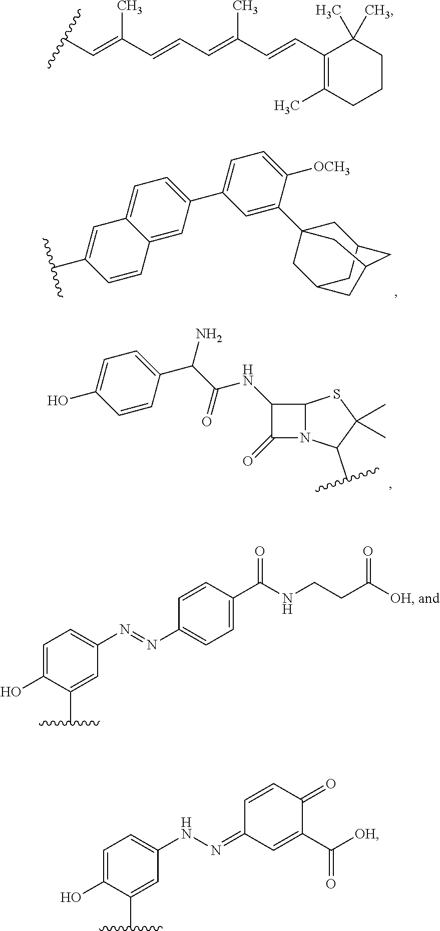

any of which may be optionally substituted by one or more R⁴; and
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

In some embodiments, the compound is a compound of Formula III:

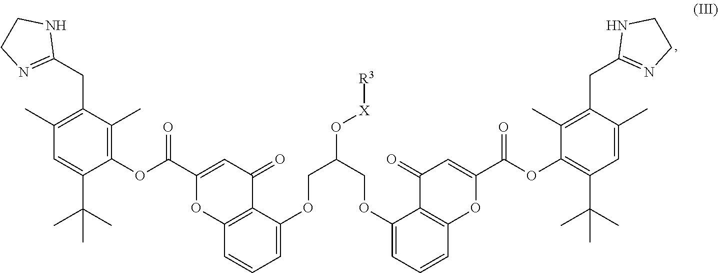

or a salt thereof, wherein:
X is —C(O)— or a bond;
R³ is hydrogen or is chosen from alkyl, cycloalkyl, heterocycloalkyl, alkenyl, alkoxy, aryl, heteroaryl,

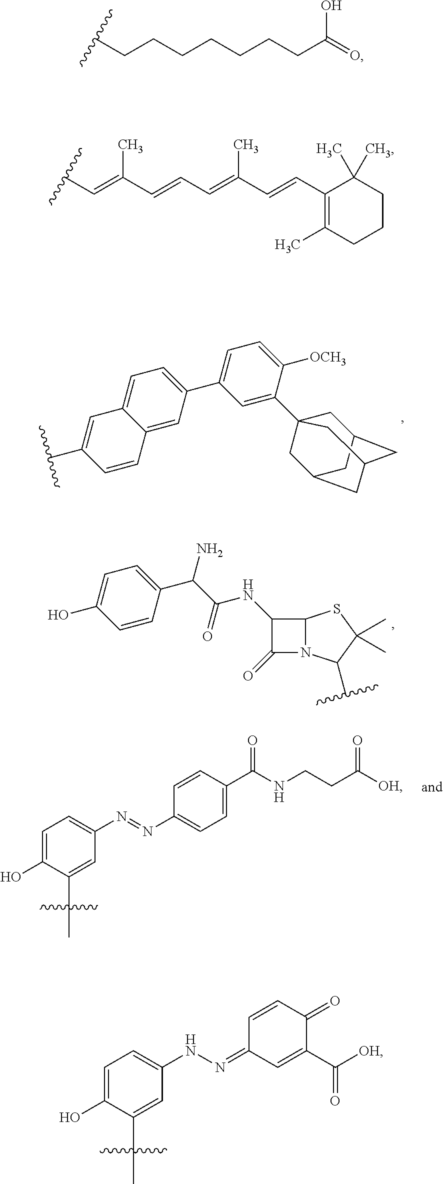 [SMILES: *CCCCCCCC(=O)O ; */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *c1cc(/N=N/c2ccc(C(=O)NCCC(=O)O)cc2)ccc1O ; *c1cc(N/N=C2\C=CC(=O)C(C(=O)O)=C2)ccc1O]

any of which may be optionally substituted by one or more R⁴; and
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

In some embodiments, the compound is a compound of Formula IV:

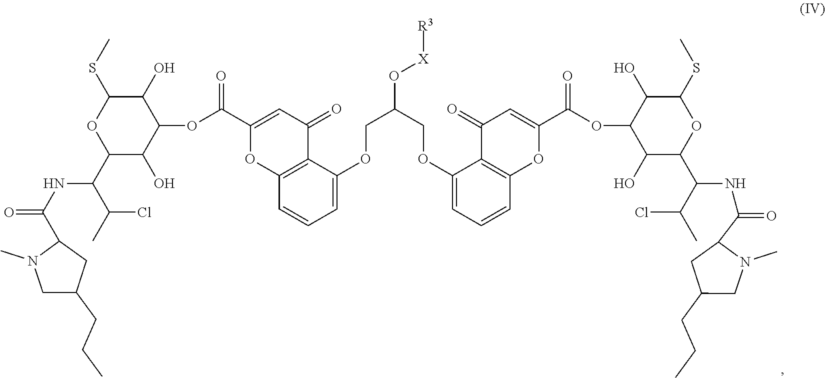

or a salt thereof, wherein:
X is —C(O)— or is a bond;
R³ is hydrogen or is chosen from alkyl, cycloalkyl, heterocycloalkyl, alkenyl, alkoxy, aryl, heteroaryl,

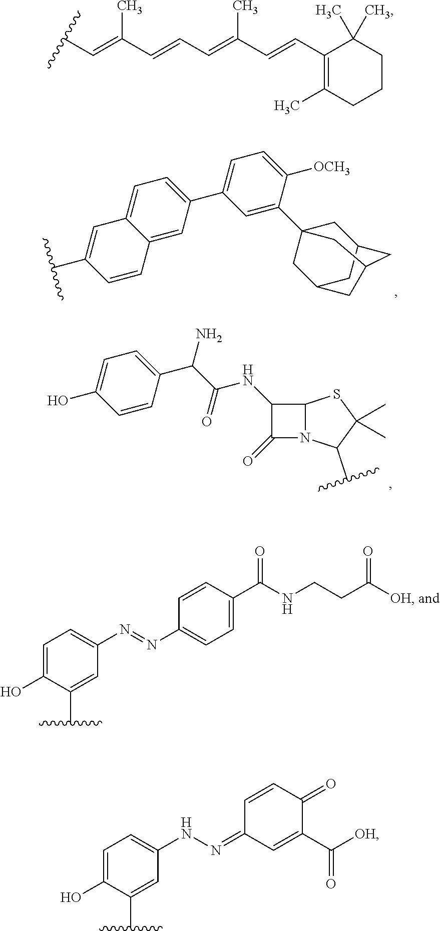

any of which may be optionally substituted by one or more R⁴; and
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

In some embodiments, the compound is a compound of Formula V:

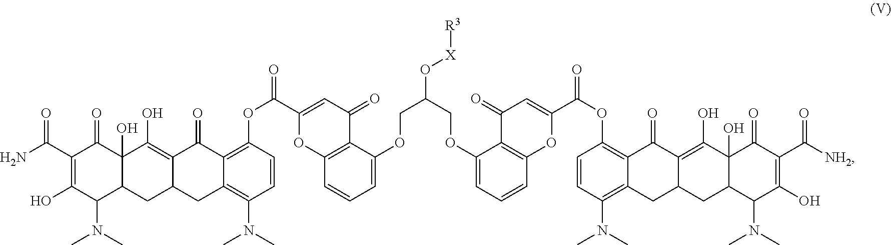

or a salt thereof, wherein:
X is —C(O)— or is a bond;
R³ is hydrogen or is chosen from alkyl, cycloalkyl, heterocycloalkyl, alkenyl, alkoxy, aryl, heteroaryl,

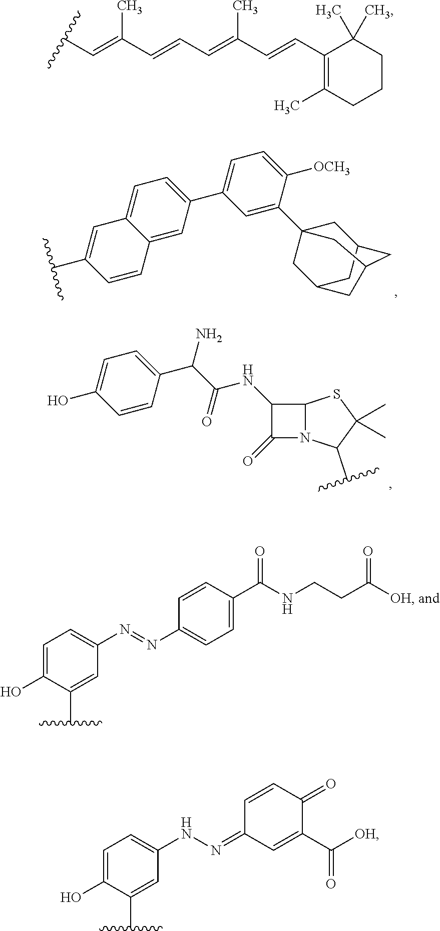

any of which may be optionally substituted by one or more R⁴; and
each R⁴ is independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

In some embodiments, X is —C(O)—.

In some embodiments, X is a bond.

In some embodiments, R³ is alkyl optionally substituted with one R⁴.

In some embodiments, R³ is hexyl.

In some embodiments, R³ is

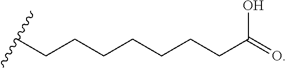 [SMILES: *CCCCCCCC(=O)O]

In some embodiments, R³ is aryl optionally substituted with one or two R⁴.

In some embodiments, R³ is phenyl optionally substituted with one or two R⁴.

In some embodiments, R³ is

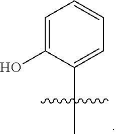 [SMILES: *c1ccccc1O]

In some embodiments, R³ is

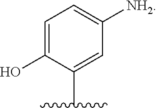 [SMILES: *c1cc(N)ccc1O]

In some embodiments, the compound is a compound of Formula VI:

or a salt thereof, wherein:

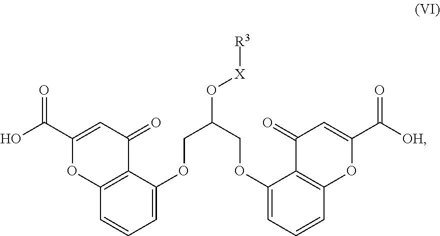

R¹ is chosen from

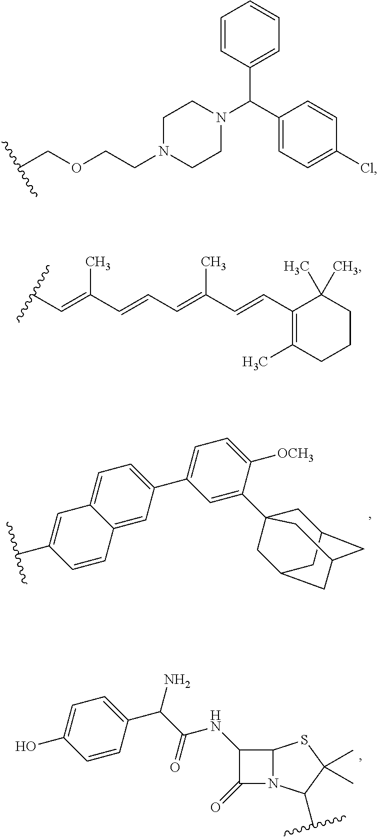 [SMILES: *COCCN1CCN(C(c2ccccc2)c2ccc(Cl)cc2)CC1 ; */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C]

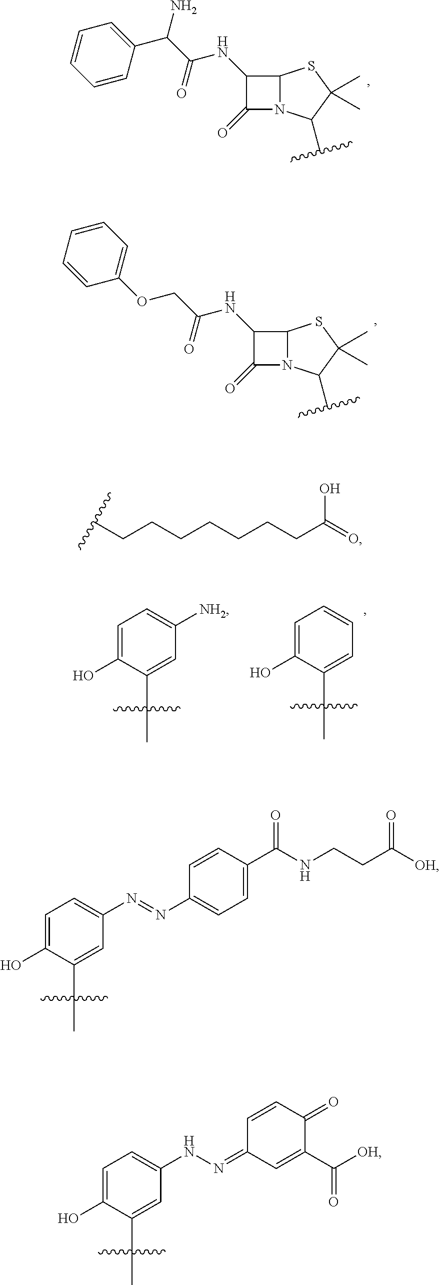

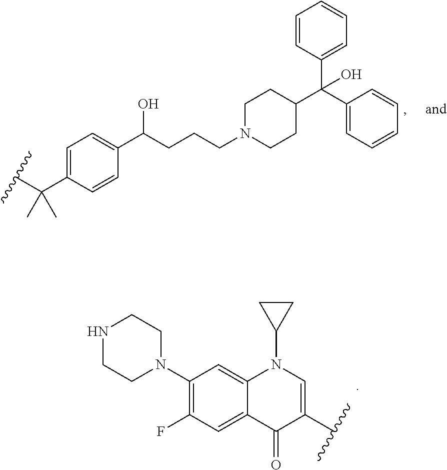 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O]

In some embodiments, the compound is chosen from

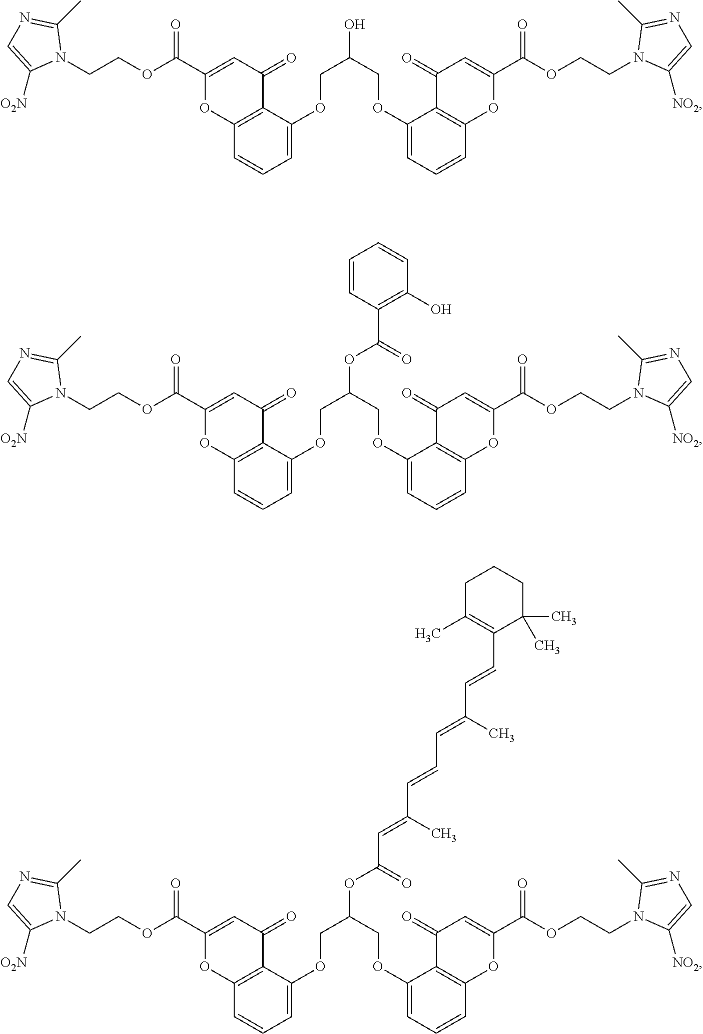 [SMILES: Cc1ncc([N+](=O)[O-])n1CCOC(=O)c1cc(=O)c2c(OCC(O)COc3cccc4oc(C(=O)OCCn5c([N+](=O)[O-])cnc5C)cc(=O)c34)cccc2o1 ; Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)c3ccccc3O)cccc2o1 ; CC1=C(/C=C/C(C)=C/C=C/C(C)=C/C(=O)OC(COc2cccc3oc(C(=O)OCCn4c([N+](=O)[O-])cnc4C)cc(=O)c23)COc2cccc3oc(C(=O)OCCn4c([N+](=O)[O-])cnc4C)cc(=O)c23)C(C)(C)CCC1]

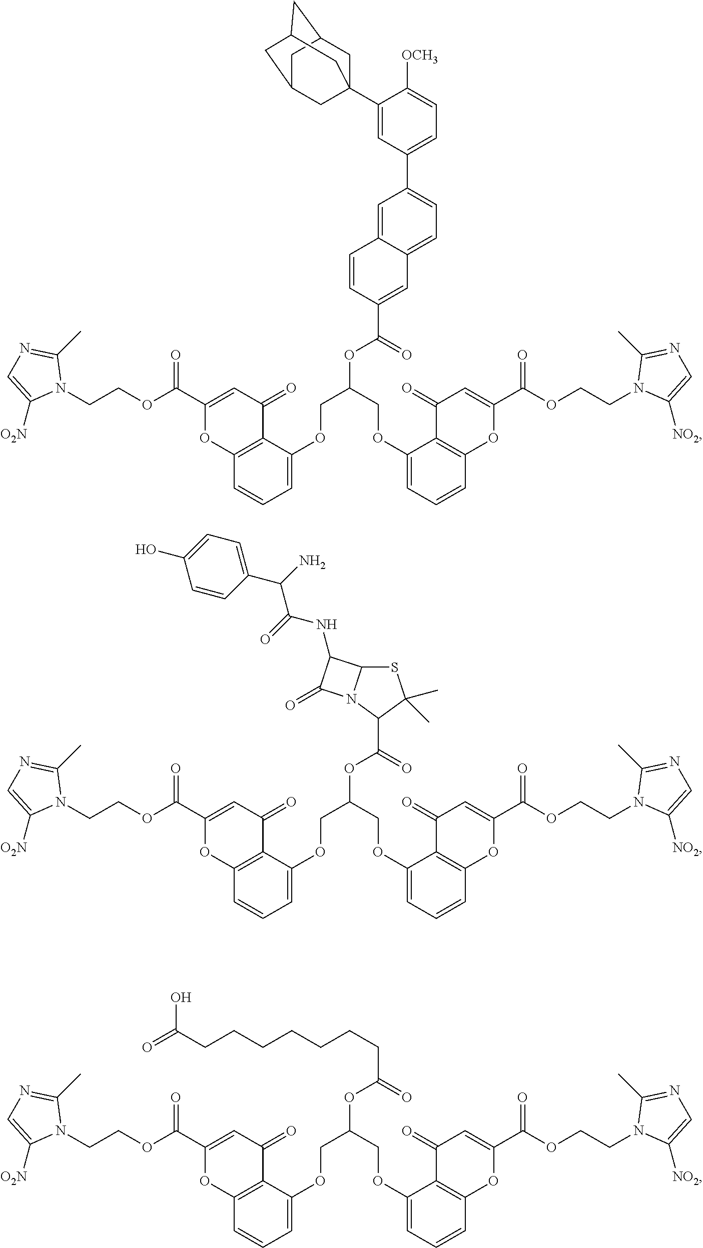

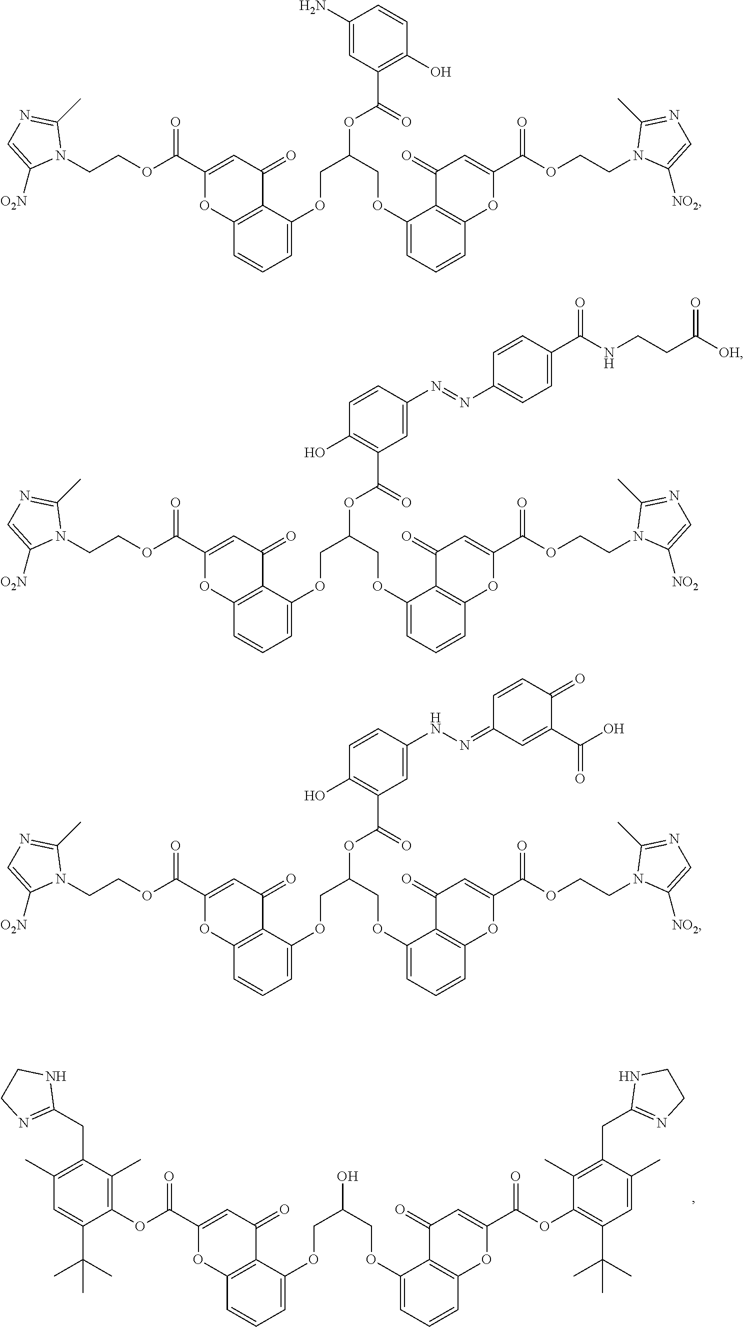

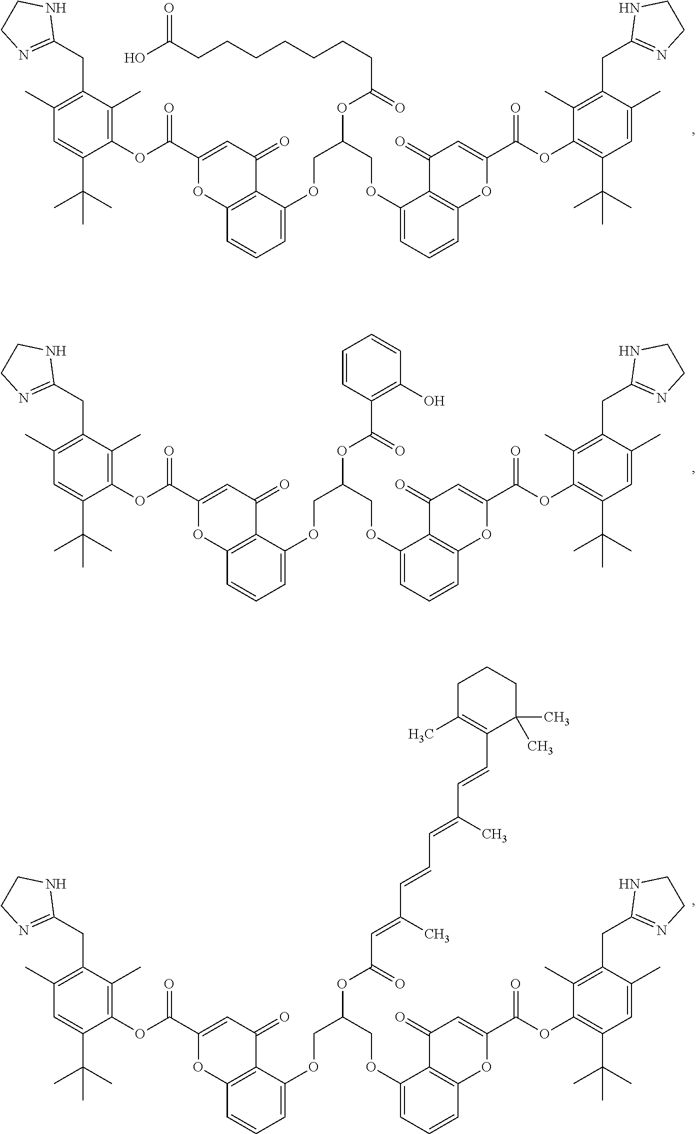 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)CCCCCCCC(=O)O)cccc3o2)c(C)c1CC1=NCCN1 ; Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)c4ccccc4O)cccc3o2)c(C)c1CC1=NCCN1 ; CC1=C(/C=C/C(C)=C/C=C/C(C)=C/C(=O)OC(COc2cccc3oc(C(=O)Oc4c(C(C)(C)C)cc(C)c(CC5=NCCN5)c4C)cc(=O)c23)COc2cccc3oc(C(=O)Oc4c(C(C)(C)C)cc(C)c(CC5=NCCN5)c4C)cc(=O)c23)C(C)(C)CCC1]

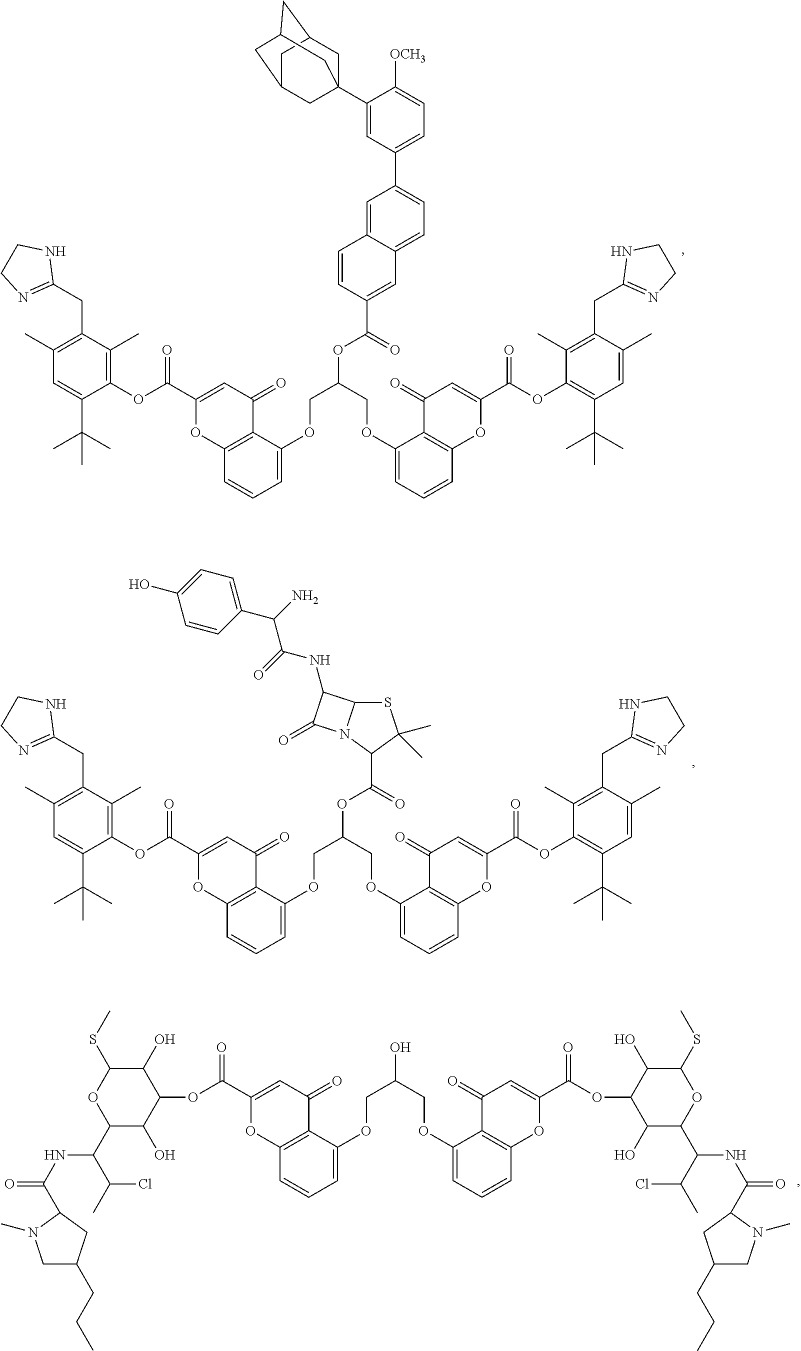

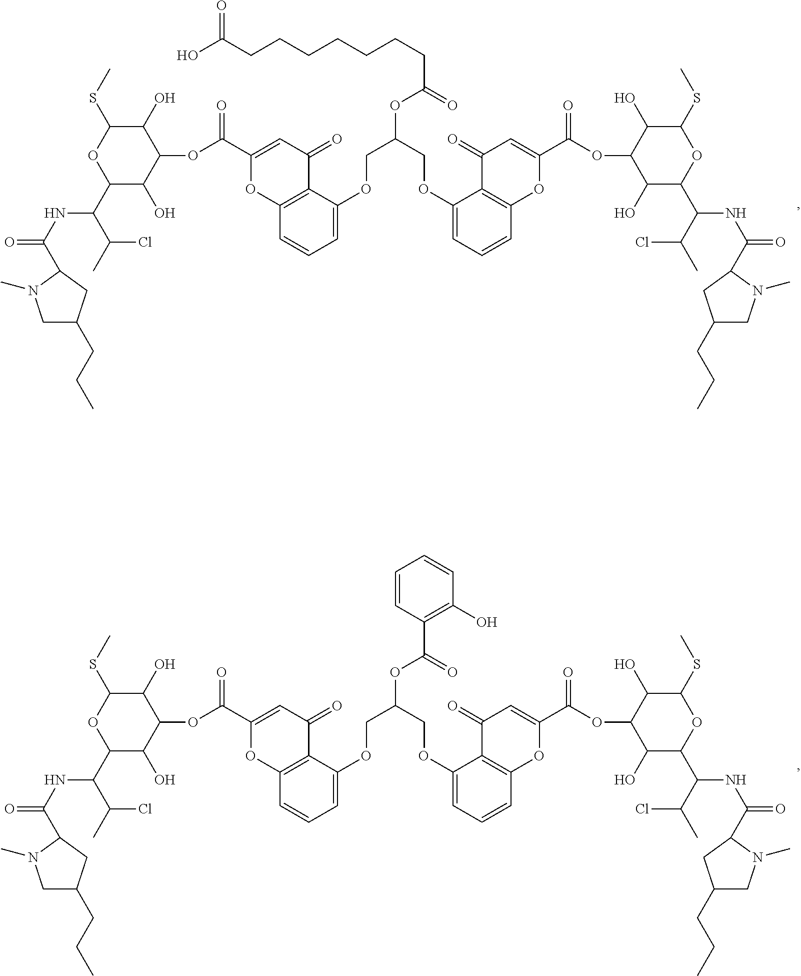

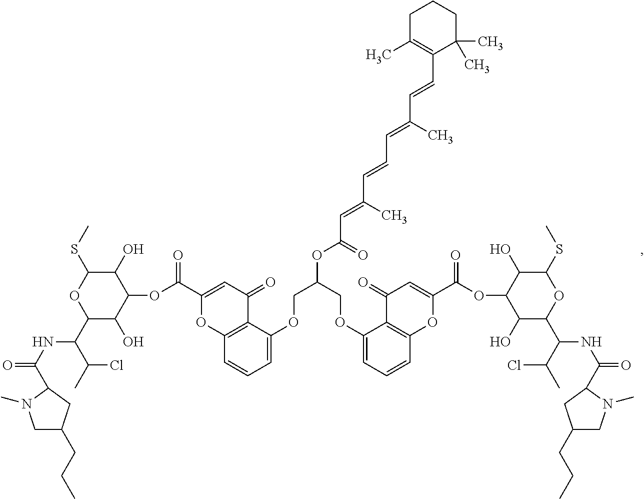

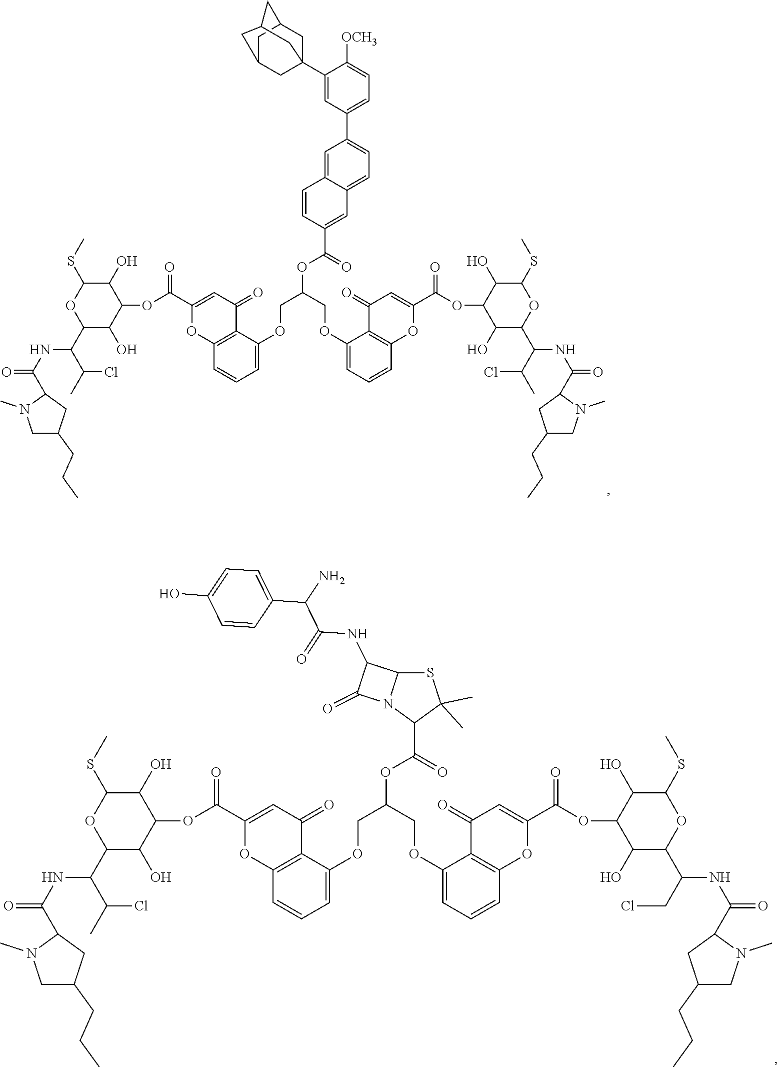

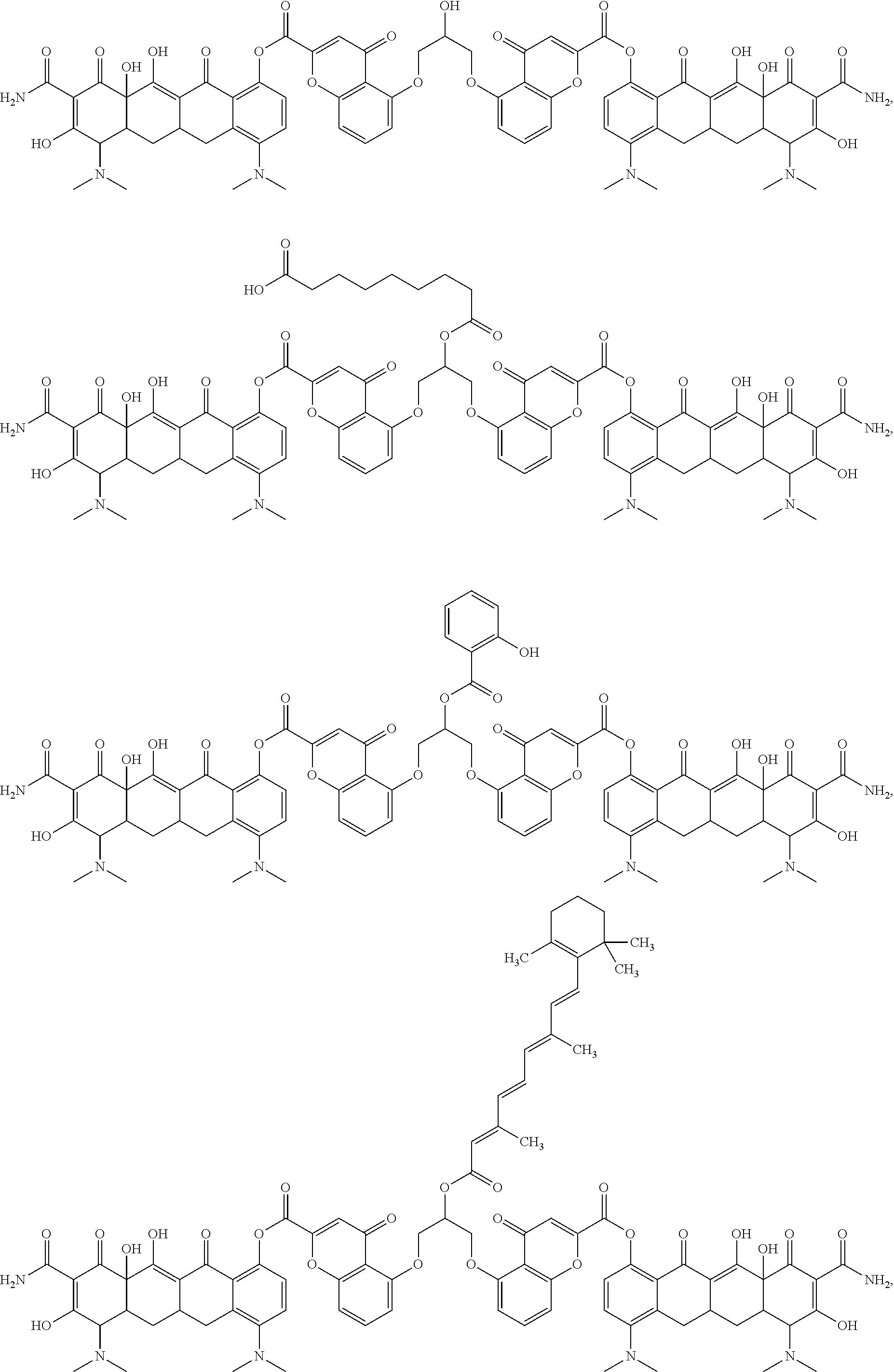

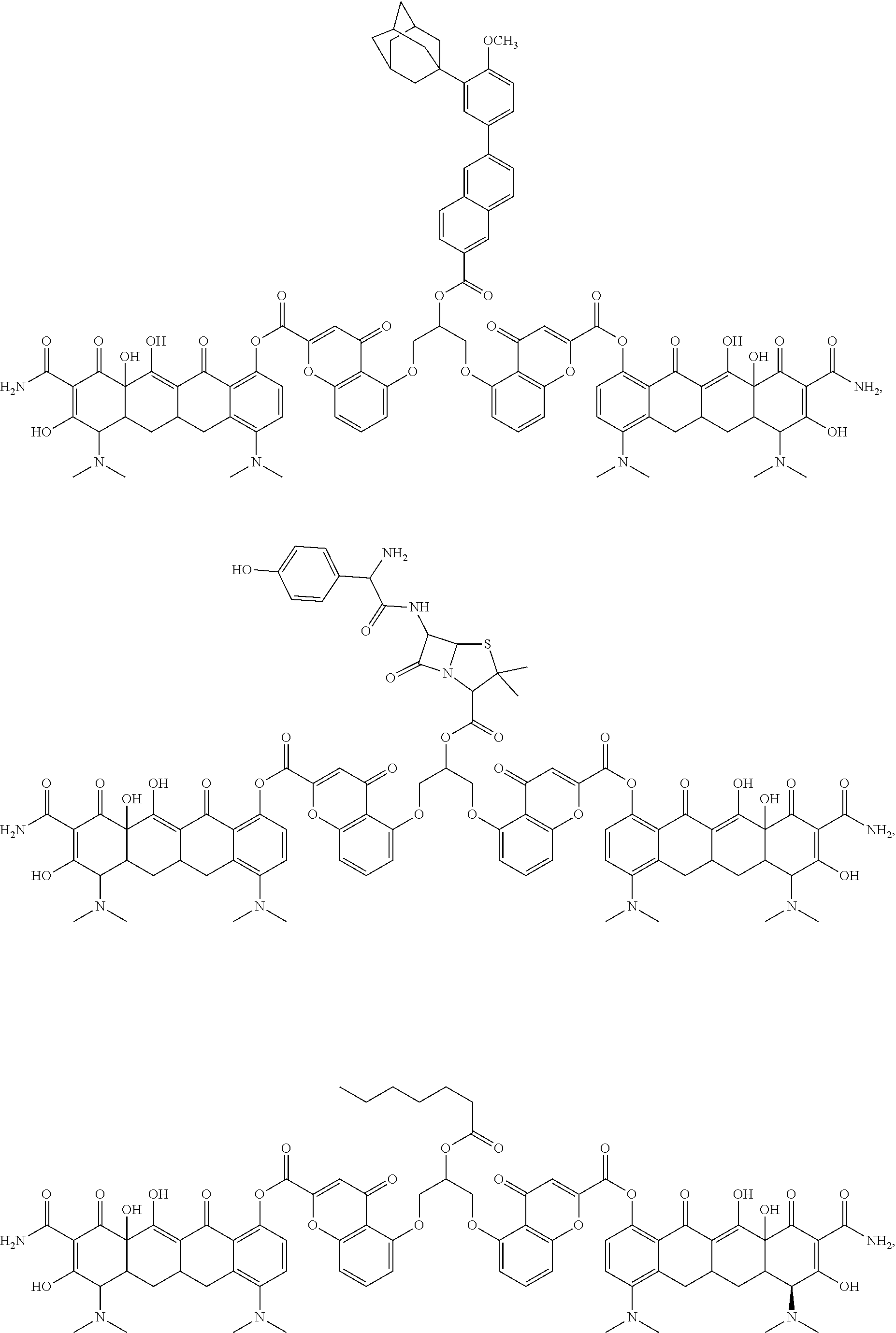

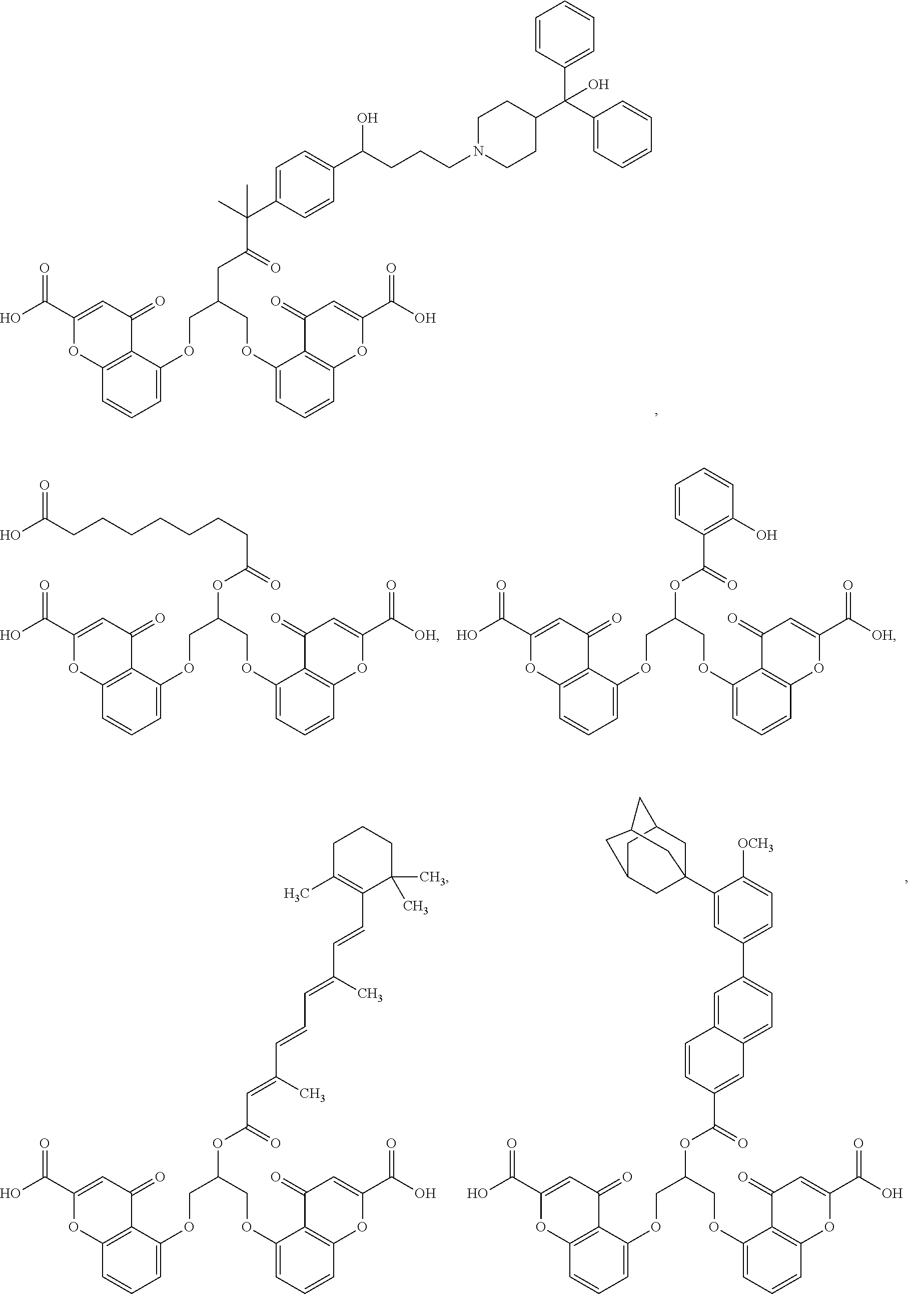

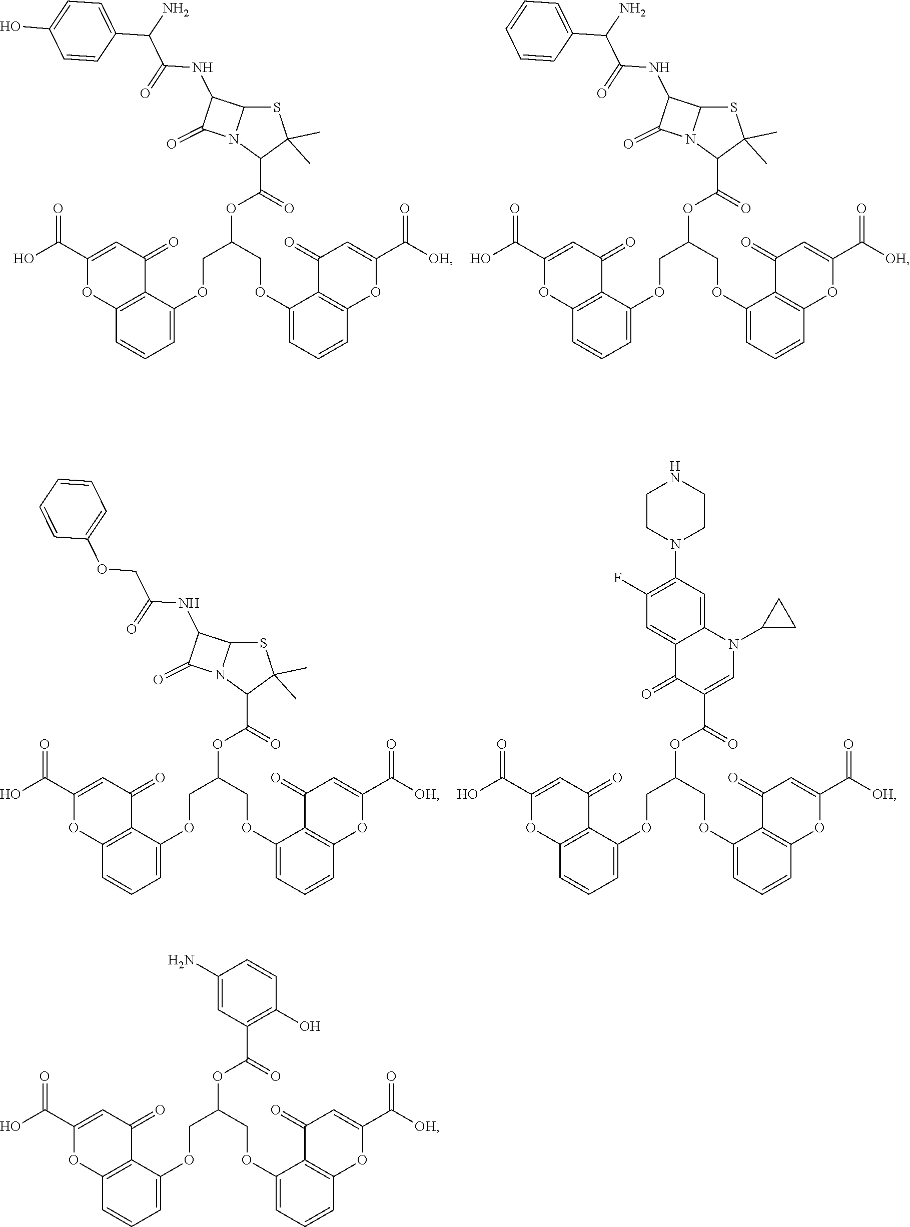

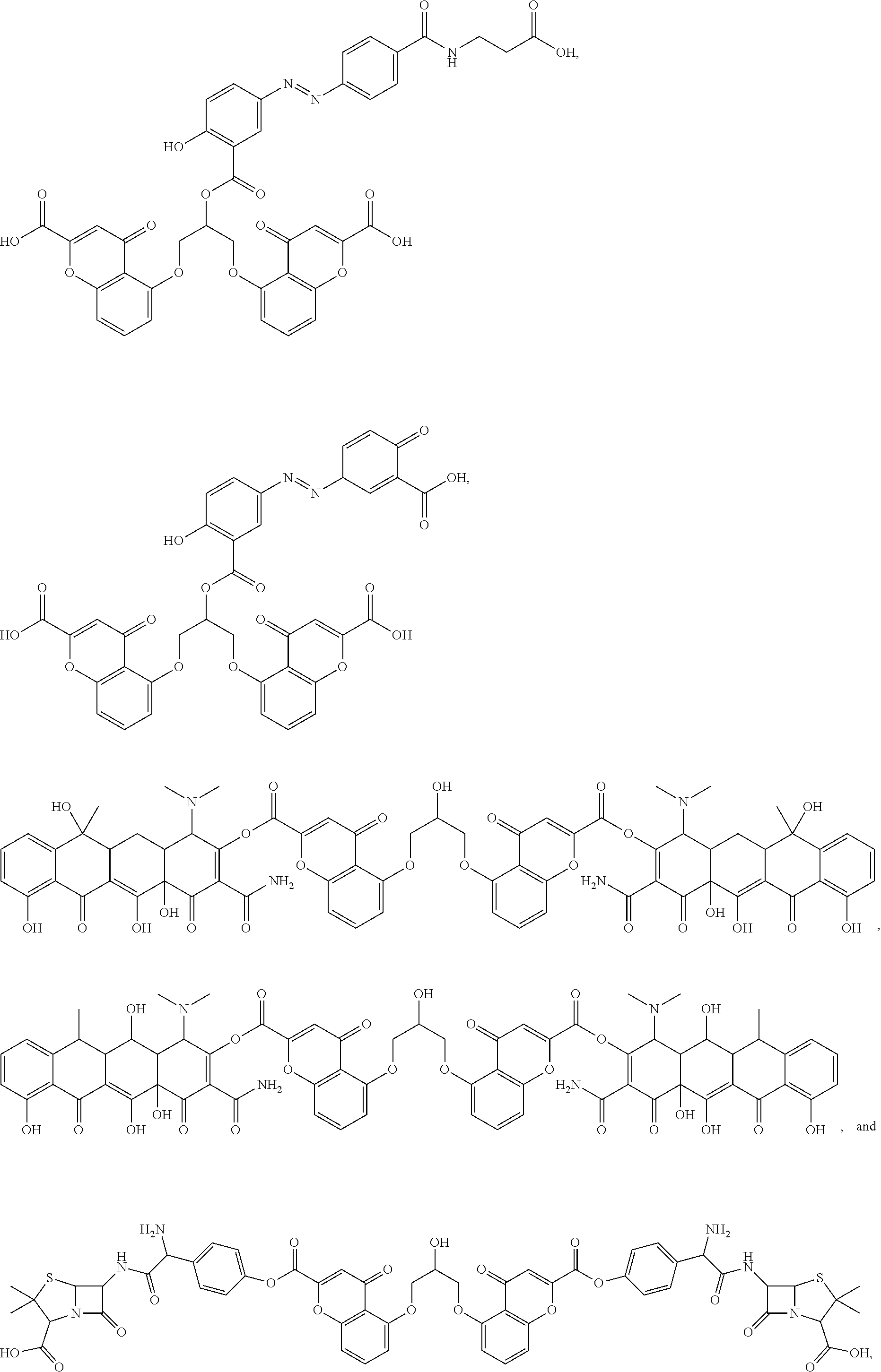

or a salt thereof.

Also provided is a pharmaceutical formulation comprising a compound as recited herein, or a salt thereof, together with a pharmaceutically acceptable carrier.

In some embodiments, the pharmaceutical formulation is formulated for topical administration.

In some embodiments, the pharmaceutical formulation additionally comprises another therapeutic agent.

Also provided is a method of inhibition of the release of chemical mediators from sensitized mast cells, comprising contacting a target cell or cells with a compound as recited herein, or a salt thereof, or a pharmaceutical composition as recited herein.

Also provided is a method of inhibition of a histaminic receptor, comprising contacting a target cell or cells with a compound as recited herein, or a salt thereof, or a pharmaceutical composition as recited herein.

In some embodiments, the histaminic receptor is the H₁ receptor.

In some embodiments, the compound or pharmaceutical composition selectively inhibits the H₁ receptor.

In some embodiments, the contacting occurs in the body of a patient.

In some embodiments, the patient is human and the target cell or cells are human.

Also provided is a method of treatment of a skin condition, comprising administering a therapeutically effective amount of a compound as recited herein, or a salt thereof, or a pharmaceutical composition as recited herein, to a patient in need thereof.

In some embodiments, the skin condition is characterized by elevated levels of mast cells, basophils, eosinophils, histamine, bradykinin, leukotriene C4, prostaglandin D2, or a combination thereof.

In some embodiments, the skin condition is characterized by elevated levels of mast cells.

In some embodiments, the skin condition is urticaria.

In some embodiments, the urticaria is chosen from chronic urticaria (CU), chronic idiopathic urticaria (CIU), or chronic spontaneous urticaria (CSU)

In some embodiments, the skin condition is chosen from atopic dermatitis, rosacea, prurigo nodularis, and acne vulgaris.

In some embodiments, the administration is topical. In some embodiments, the administration directly applies to disease-induced skin lesions on the patient.

Also provided is method of treatment of an inflammatory disease chosen from mastocytosis, inflammatory bowel disease (IBD), Crohn's disease, and ulcerative colitis (UC), comprising administering a therapeutically effective amount of a compound as recited herein, or a salt thereof, or a pharmaceutical composition as recited herein, to a patient in need thereof.

In some embodiments, the skin condition is treatable with anti-inflammatory agents, topical antiseptics, topical or oral antibiotics, anti-inflammatory agents, keratin softener, vasoconstrictors, retinoids, or retinoid-like drugs. In some embodiments, the inflammatory disease is treatable with mast cell stabilizers, anti-inflammatory agents, or oral antibiotics.

The compounds disclosed herein can exist as pharmaceutically acceptable salts. The present disclosure includes salts of the compounds listed herein, including acid addition salts. Suitable salts include those formed with both organic and inorganic acids. Such acid addition salts will normally be pharmaceutically acceptable. However, salts of non-pharmaceutically acceptable salts may be useful in preparing and purifying the compound in question. Basic addition salts may also be formed and be pharmaceutically acceptable. For a complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein. The salts can be prepared during the final isolation and purification of the compounds or separately by reacting the appropriate compound as the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids that can form pharmaceutically acceptable addition salts include inorganic acids such as hydrochloric, hydrobromic, sulfuric, and phosphoric, and organic acids such as oxalic, maleic, succinic, and citric. The coordination of the compounds with an alkali metal or alkaline earth ion can also form salts. Hence, the present disclosure contemplates sodium, potassium, magnesium, and calcium salts of the compounds disclosed herein and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines helpful in forming base addition salts include ethylenediamine, ethanolamine, diethanolamine, piperidine, and piperazine.

### Pharmaceutical Compositions

While it may be possible for the compounds, and pharmaceutically acceptable salts thereof, of the subject disclosure to be administered as the raw chemical, it is also possible to present them as a pharmaceutical formulation.

Also provided is a pharmaceutical formulation comprising a compound as disclosed herein, or a pharmaceutically acceptable salt thereof, together with a pharmaceutically acceptable carrier. The formulations may conveniently be presented in unit dosage form and prepared by any methods well-known in pharmacy. Typically, these methods include the step of bringing into association a compound, or pharmaceutically acceptable salts thereof, of the subject disclosure or a pharmaceutically acceptable salt thereof (“active ingredient”) with the carrier, which constitutes one or more accessory ingredients. In general, the formulations are prepared by uniformly and intimately associating the active ingredient with liquid carriers or finely divided solid carriers, or both, and then, if necessary, shaping the product into the desired formulation.

Preferred unit dosage formulations contain an effective dose, or an appropriate fraction thereof, of the active ingredient.

Compounds, or pharmaceutically acceptable salts thereof, may be administered at a dose of from 0.1 to 500 mg/kg per day. The dose range for adult humans is generally from 5 mg to 2 g/day. The amount of active ingredient that may be combined with the carrier materials to produce a single dosage form will vary depending upon the host treated and the particular mode of administration.

The compounds, or pharmaceutically acceptable salts thereof, can be administered in various modes. In some embodiments, the pharmaceutical formulation is formulated for oral administration.

In certain instances, it may be appropriate to administer at least one compound described herein (or a pharmaceutically acceptable salt thereof) in combination with another therapeutic agent. By way of example only, if one side effect experienced by a patient upon receiving one compound herein, or a pharmaceutically acceptable salt thereof, is hypertension, then it may be appropriate to administer an anti-hypertensive agent in combination with the initial therapeutic agent. Or, by way of example only, the therapeutic effectiveness of one compound described herein, or pharmaceutically acceptable salts thereof, may be enhanced by administration of an adjuvant (i.e., by itself, the adjuvant may only have minimal therapeutic benefit, but in combination with another therapeutic agent, the overall therapeutic benefit to the patient is enhanced). Or, by way of example, only, the benefit experienced by a patient may be increased by administering at least one compound described herein, or pharmaceutically acceptable salts thereof, with another therapeutic agent (which also includes a therapeutic regimen) that also has therapeutic benefit. Regardless of the disease, disorder, or condition being treated, the overall benefit experienced by the patient may be additive of the two therapeutic agents, or the patient may experience a synergistic benefit.

The multiple therapeutic agents (at least one compound disclosed herein or a pharmaceutically acceptable salt thereof) may be administered in any order or even simultaneously. If simultaneously, the multiple therapeutic agents may be provided in a single, unified form or multiple forms (by example only, either as a single pill or as two separate pills). One of the therapeutic agents may be given in multiple doses, or both may be given in multiple doses. If not simultaneous, the timing between the multiple doses may vary from a few minutes to four weeks.

In certain embodiments, the lipophilic and esterase-cleavable mutual prodrug is used for oral oil-based formulations to treat mastocytosis. In the case of oral formulations, the lipophilic mutual prodrug is diluted in pharmaceutical oil(s) and encapsulated in hard-shell gelatin pills (in some cases, the pills can be enteric-coated). In the intestine, the lipophilic mutual prodrug is absorbed with the oil and then hydrolyzed by endogenous esterases to Cromolyn sodium and parental APIs.

In certain embodiments, the lipophilic and esterase-cleavable mutual prodrug is used for oral oil-based formulations to treat inflammatory bowel disease and ulcerative colitis. For oral formulations, the lipophilic mutual prodrug is diluted in pharmaceutical oil(s) and encapsulated in hard-shell gelatin pills (in some cases, the pills can be enteric-coated). In the intestine, the lipophilic mutual prodrug is absorbed with the oil and then cleaved by endogenous esterases to Cromolyn sodium and parental APIs.

In certain embodiments, the lipophilic and esterase-cleavable mutual prodrug is used for inhalation formulation in the form of oil-based water emulsion to treat chronic asthma.

In certain embodiments, the lipophilic and esterase-cleavable mutual prodrug is used as an ophthalmic solution (oil-based water emulsion) for use in rhinitis and other ocular conditions, particularly for the treatment of vernal keratoconjunctivitis (vernal catarrh, spring catarrh), allergic conjunctivitis, and hay fever.

Certain other additional components, such as emulsifying and stiffening agents, may provide, e.g., stability and desired consistency to the compositions. In some embodiments, the emulsifying agents are chosen from poloxamers, lecithin, carbomers, polyoxyethylene ethers, fatty acid esters, and stearates. In some embodiments, the poloxamer is Pluronic F-127. Typical stiffening agents useful in the present compositions include long-chain fatty alcohols and long-chain fatty alcohol esters.

In addition to any of the above additional components, one or more of the agents discussed above for the mutual prodrug of the present disclosure may be included as a free component (e.g., not covalently bonded to cromoglicic acid or in addition to a composition including the mutual prodrug) in any of the compositions of the present disclosure. Furthermore, as these agents are not covalently linked to cromoglicic acid, the choice of this compound present as a free agent is not limited in this way.

In some embodiments, the mutual prodrug is present in the composition in an amount of at least about 1% (w/w) or at least about 10% (w/w). Further, the mutual prodrug is typically present in the compositions in an amount of no greater than about 75% (w/w) and preferably no greater than about 40% (w/w).

In some embodiments, topical compositions containing the mutual prodrug may be prepared from various combinations of additional components. In some embodiments, the additional components are chosen from isopropyl palmitate, isopropyl myristate, a stiffening agent like long-chain fatty alcohols, long-chain fatty alcohol esters, waxes like spermaceti, nonionic gelling/emulsifiers like poloxamers, USP approved antimicrobial agents, cetyl alcohol, cetearyl alcohol, cocoa butter, isopropyl palmitate, lanolin, liquid paraffin, polyethylene glycols, shea butter, silicone oils, stearic acid, stearyl alcohol, petrolatum, dimethicone, castor oil, safflower oil, mineral oil, and other common emollients. In some embodiments, the additional components are selected to provide desired characteristics to the compositions, including but not limited to enhanced permeability and/or improved bioavailability.

In some embodiments, the mutual prodrug is administered to a patient topically and/or transdermally via local application to the skin or other external or internal membranes. In some embodiments, the mutual prodrug is administered orally, such as in the form of tablets, powders, capsules, suspensions, emulsions, gels, etc.

Further embodiments include the embodiments disclosed in the following Schemes and Examples, which are not to be construed as limiting in any way.

### Schemes

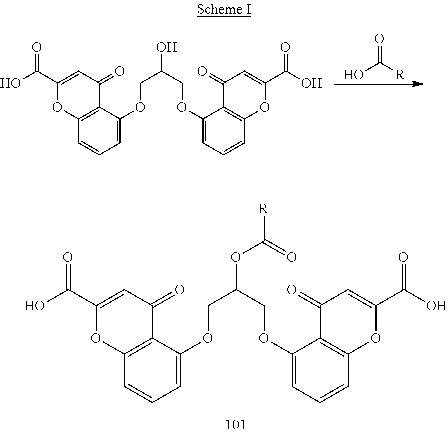

Referring to Scheme I, to a solution of cromoglicic acid in an organic solvent, such as toluene, is added a compound of R—CO₂H (R=any of R¹, R², or R³ as described herein), and a strong acid, such as p-toluenesulfonic acid. In some embodiments, the mixture is under an inert atmosphere. The mixture is stirred, optionally at elevated temperatures. In some embodiments, the mixture is stirred for 1-12 h. The product, a compound of Formula 101, is isolated and purified using methods known in the art. Individual enantiomers can be separated using methods known in the art, such as chiral chromatography.

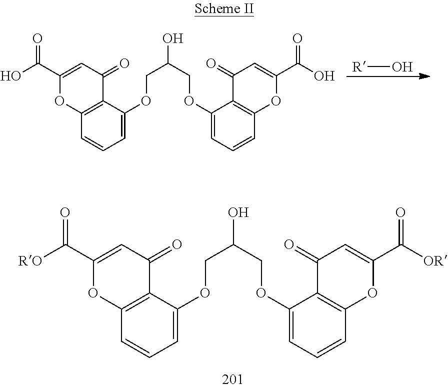

Referring to Scheme II, to a solution of cromoglicic acid in an organic solvent, such as toluene, is added a compound of R′—OH (R′=any of R¹, R², or R³ as described herein), and a strong acid, such as p-toluenesulfonic acid. In some embodiments, the mixture is under an inert atmosphere. The mixture is stirred, optionally at elevated temperatures. In some embodiments, the mixture is stirred for 1-12 h. The product, a compound of Formula 201, is isolated and purified using methods known in the art. Individual enantiomers can be separated using methods known in the art, such as chiral chromatography.

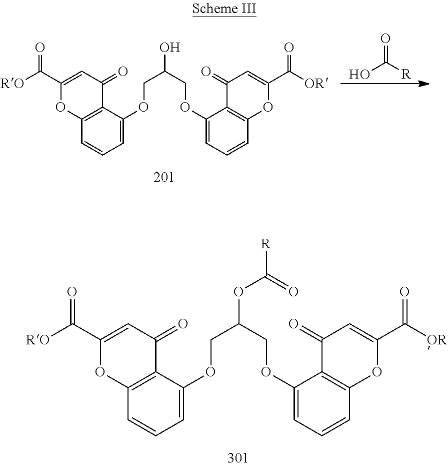

Referring to Scheme III, to a solution of a compound of Formula 201 (R′=any of R¹, R², or R³ as described herein) in an organic solvent, such as toluene, is added a compound of R—CO₂H (R=any of R¹, R², or R³ as described herein), and a strong acid, such as p-toluenesulfonic acid. In some embodiments, the mixture is under an inert atmosphere. The mixture is stirred, optionally at elevated temperatures. In some embodiments, the mixture is stirred for 1-12 h. The product, a compound of Formula 301, is isolated and purified using methods known in the art. Individual enantiomers can be separated using methods known in the art, such as chiral chromatography.

### Example 1

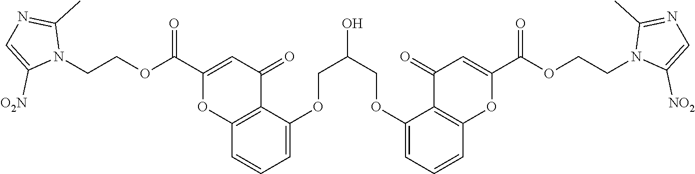 [SMILES: Cc1ncc([N+](=O)[O-])n1CCOC(=O)c1cc(=O)c2c(OCC(O)COc3cccc4oc(C(=O)OCCn5c([N+](=O)[O-])cnc5C)cc(=O)c34)cccc2o1]

bis(2-(2-methyl-5-nitro-1H-imidazol-1-yl)ethyl) 5,5′-((2-hydroxypropane-1,3-diyl)bis(oxy)) bis(4-oxo-4H-chromene-2-carboxylate) In a flask, 11.7 g of cromoglicic acid, 18.9 g of metronidazole, and 0.17 g of p-toluenesulfonic acid were charged under a nitrogen atmosphere. 50 mL of toluene was added, and the mixture was refluxed at 125° C. for 5 hours. Water formed during the reaction was separated azeotropically with a Dean-Stark apparatus. The reaction was monitored by thin-layer chromatography (TLC). After completion of the reaction according to TLC, the mixture was cooled to 25° C. and quenched with water. 100 mL of ethyl acetate was added, and the organic layer was separated. The aqueous layer was washed twice with ethyl acetate (50 mL each). The organic layers were combined, concentrated, and dried over anhydrous sodium sulfate to obtain a crude product. The crude product was purified using silica gel flash chromatography using a solvent system containing 5-10% ethyl acetate in hexanes to obtain the title compound (dimetronidazole cromoglicate) in 67% yield and 99% purity.

### Example 2

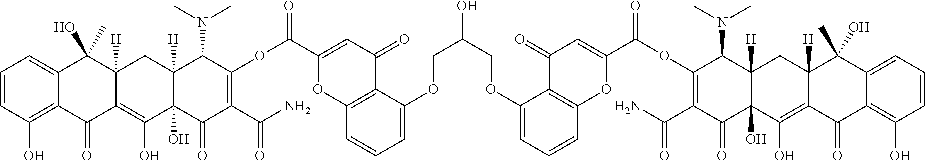 [SMILES: CN(C)[C@@H]1C(OC(=O)c2cc(=O)c3c(OCC(O)COc4cccc5oc(C(=O)OC6=C(C(N)=O)C(=O)[C@@]7(O)C(O)=C8C(=O)c9c(O)cccc9[C@@](C)(O)[C@H]8C[C@H]7[C@@H]6N(C)C)cc(=O)c45)cccc3o2)=C(C(N)=O)C(=O)[C@@]2(O)C(O)=C3C(=O)c4c(O)cccc4[C@@](C)(O)[C@H]3C[C@@H]12]

### Bis((1S,4aS,11S,11aS,12aS)-3-Carbamoyl-1-(Dimethylamino)-4a,5,7,11-Tetrahydroxy-11-Methyl-4,6-Dioxo-1,4,4a,6,11,11a,12,12a-Octahydrotetracen-2-Yl) 5,5′-((2-Hydroxypropane-1,3-Diyl)Bis(Oxy)) Bis(4-Oxo-4H-Chromene-2-Carboxylate)

In a flask, 11.7 g of cromoglicic acid, 24.6 g of tetracycline, and 0.17 g of p-toluenesulfonic acid were charged under a nitrogen atmosphere. 50 mL of toluene was added, and the mixture was refluxed at 125° C. for 5 hours. Water formed during the reaction was separated azeotropically with a Dean-Stark apparatus. The reaction was monitored by thin-layer chromatography (TLC). After completion of the reaction according to TLC, the mixture was cooled to 25° C. and quenched with water. 100 mL of ethyl acetate was added, and the organic layer was separated. The aqueous layer was washed twice with ethyl acetate (50 mL each). The organic layers were combined, concentrated, and dried over anhydrous sodium sulfate to obtain a crude product. The crude product was purified using silica gel flash chromatography using a solvent system containing 5-10% ethyl acetate in hexanes to obtain the title compound (ditetracycline cromoglicate) in 74% yield and 99% purity.

### Example 3

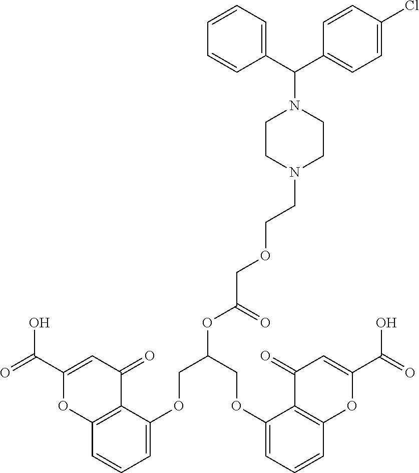 [SMILES: O=C(COCCN1CCN(C(c2ccccc2)c2ccc(Cl)cc2)CC1)OC(COc1cccc2oc(C(=O)O)cc(=O)c12)COc1cccc2oc(C(=O)O)cc(=O)c12]

### 5,5′-((2-(2-(2-(4-((4-Chlorophenyl) (Phenyl)Methyl) Piperazin-1-Yl) Ethoxy) Acetoxy) Propane-1,3-Diyl)Bis(Oxy)) Bis(4-Oxo-4H-Chromene-2-Carboxylic Acid

In a flask, 11.7 g of cromoglicic acid, 10.7 g of cetirizine, and 0.17 g of p-toluenesulfonic acid were charged under a nitrogen atmosphere. 50 mL of toluene was added, and the mixture was refluxed at 125° C. for 5 hours. Water formed during the reaction was separated azeotropically with a Dean-Stark apparatus. The reaction was monitored by thin-layer chromatography (TLC). After completion of the reaction according to TLC, the mixture was cooled to 25° C. and quenched with water. 100 mL of ethyl acetate was added, and the organic layer was separated. The aqueous layer was washed twice with ethyl acetate (50 mL each). The organic layers were combined, concentrated, and dried over anhydrous sodium sulfate to obtain a crude product. The crude product was purified using silica gel flash chromatography using a solvent system containing 5-10% ethyl acetate in hexanes to obtain the title compound (cetirizine cromoglicate) in 67% yield and 99% purity.

### Example 4

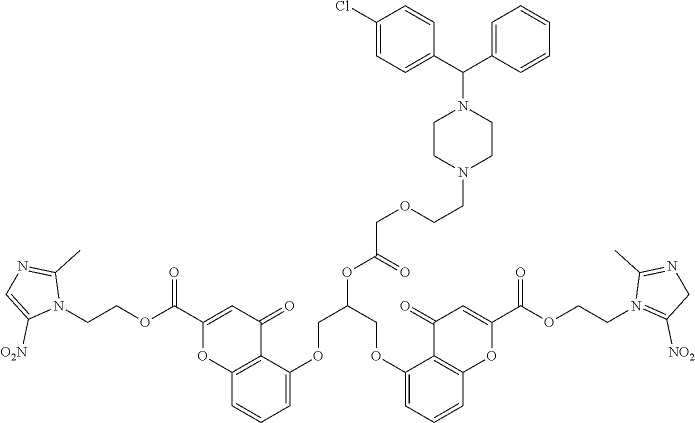

Bis(2-(2-methyl-5-nitro-1H-imidazol-1-yl)ethyl) 5,5′-((2-(2-(2-(4-((4-chlorophenyl) (phenyl)methyl) piperazin-1-yl) ethoxy) acetoxy) propane-1,3-diyl)bis(oxy)) bis(4-oxo-4H-chromene-2-carboxylate) In a flask, 11.7 g of cetirizine, 21.3 g of bis(2-(2-methyl-5-nitro-1H-imidazol-1-yl)ethyl) 5,5′-((2-hydroxypropane-1,3-diyl)bis(oxy)) bis(4-oxo-4H-chromene-2-carboxylate) (Example 1), and 0.17 g of p-toluenesulfonic acid were charged under a nitrogen atmosphere. 50 mL of toluene was added, and the mixture was refluxed at 125° C. for 5 hours. Water formed during the reaction was separated azeotropically with a Dean-Stark apparatus. The reaction was monitored by thin-layer chromatography (TLC). After completion of the reaction according to TLC, the mixture was cooled to 25° C. and quenched with water. 100 mL of ethyl acetate was added, and the organic layer was separated. The aqueous layer was washed twice with ethyl acetate (50 mL each). The organic layers were combined, concentrated, and dried over anhydrous sodium sulfate to obtain a crude product. The crude product was purified using silica gel flash chromatography using a solvent system containing 5-10% ethyl acetate in hexanes to obtain the title compound (cetirizine dimetronidazole cromoglicate) in 87% yield and 99% purity.

The following Examples were synthesized with procedures similar to those disclosed herein and can generally be made using methods disclosed herein from the appropriate starting materials.

TABLE 1
Example Compounds.

| Ex- | |
|---|---|
| am- | |
| ple | Structure |
| 5 | 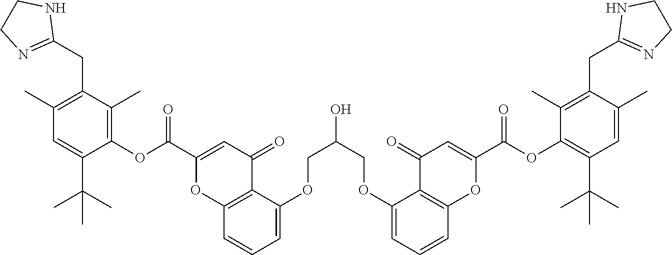 [SMILES: Cc1cc(C(C)(C)C)c(OC(=O)c2cc(=O)c3c(OCC(O)COc4cccc5oc(C(=O)Oc6c(C(C)(C)C)cc(C)c(CC7=NCCN7)c6C)cc(=O)c45)cccc3o2)c(C)c1CC1=NCCN1] |
| 6 | 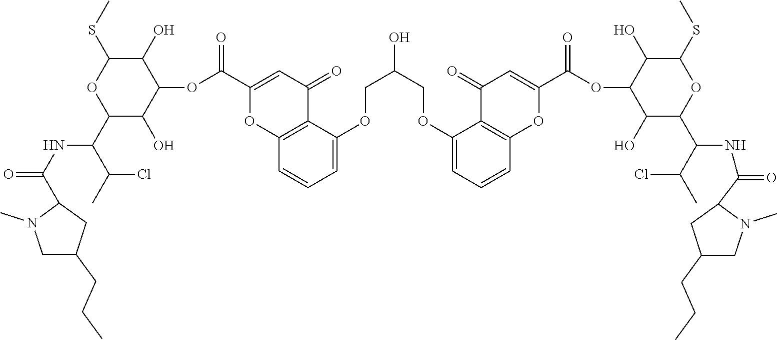 |
| 7 | 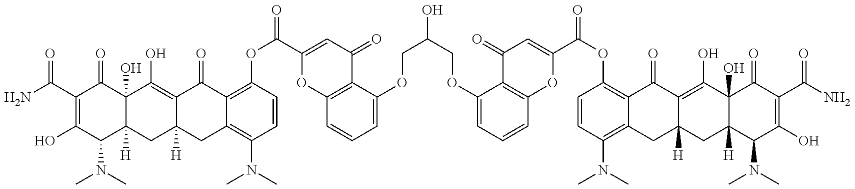 [SMILES: CN(C)c1ccc(OC(=O)c2cc(=O)c3c(OCC(O)COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)cccc3o2)c2c1C[C@H]1C[C@H]3[C@H](N(C)C)C(O)=C(C(N)=O)C(=O)[C@@]3(O)C(O)=C1C2=O] |
| 8 | 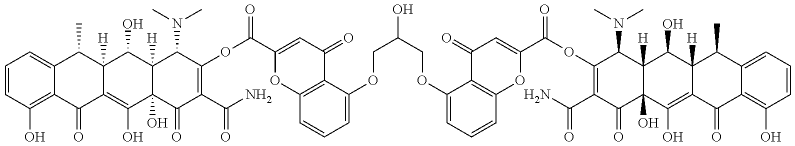 [SMILES: C[C@H]1c2cccc(O)c2C(=O)C2=C(O)[C@]3(O)C(=O)C(C(N)=O)=C(OC(=O)c4cc(=O)c5c(OCC(O)COc6cccc7oc(C(=O)OC8=C(C(N)=O)C(=O)[C@@]9(O)C(O)=C%10C(=O)c%11c(O)cccc%11[C@H](C)[C@H]%10[C@H](O)[C@H]9[C@@H]8N(C)C)cc(=O)c67)cccc5o4)[C@@H](N(C)C)[C@@H]3[C@@H](O)[C@@H]21] |
| 9 | 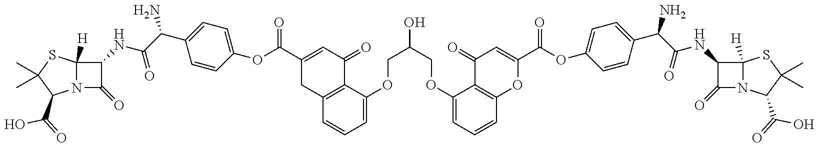 |
| 10 | 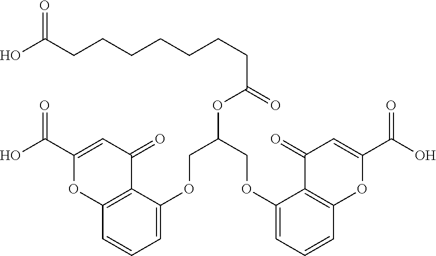 [SMILES: O=C(O)CCCCCCCC(=O)OC(COc1cccc2oc(C(=O)O)cc(=O)c12)COc1cccc2oc(C(=O)O)cc(=O)c12] |
| 11 | 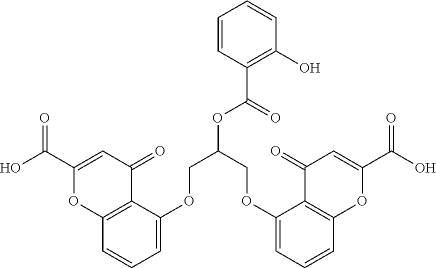 [SMILES: O=C(O)c1cc(=O)c2c(OCC(COc3cccc4oc(C(=O)O)cc(=O)c34)OC(=O)c3ccccc3O)cccc2o1] |
| 12 | 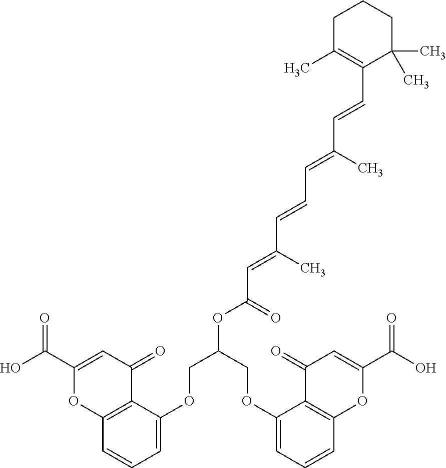 [SMILES: CC1=C(/C=C/C(C)=C/C=C/C(C)=C/C(=O)OC(COc2cccc3oc(C(=O)O)cc(=O)c23)COc2cccc3oc(C(=O)O)cc(=O)c23)C(C)(C)CCC1] |
| 13 | 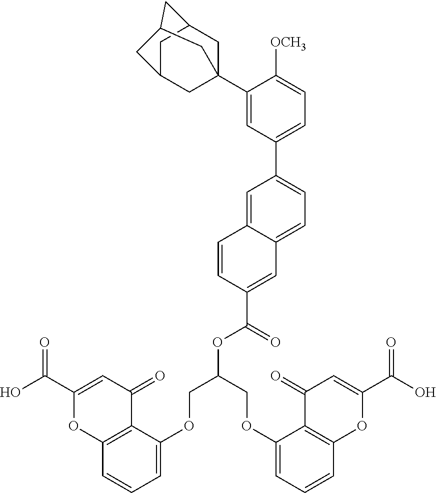 [SMILES: COc1ccc(-c2ccc3cc(C(=O)OC(COc4cccc5oc(C(=O)O)cc(=O)c45)COc4cccc5oc(C(=O)O)cc(=O)c45)ccc3c2)cc1C12CC3CC(CC(C3)C1)C2] |
| 14 | 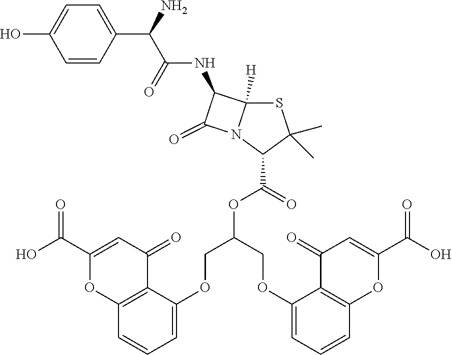 [SMILES: CC1(C)S[C@@H]2[C@H](NC(=O)[C@H](N)c3ccc(O)cc3)C(=O)N2[C@H]1C(=O)OC(COc1cccc2oc(C(=O)O)cc(=O)c12)COc1cccc2oc(C(=O)O)cc(=O)c12] |
| 15 | 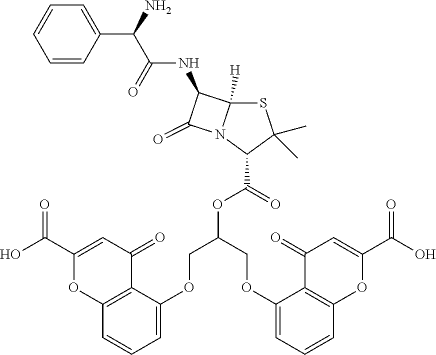 [SMILES: CC1(C)S[C@@H]2[C@H](NC(=O)[C@H](N)c3ccccc3)C(=O)N2[C@H]1C(=O)OC(COc1cccc2oc(C(=O)O)cc(=O)c12)COc1cccc2oc(C(=O)O)cc(=O)c12] |
| 16 | 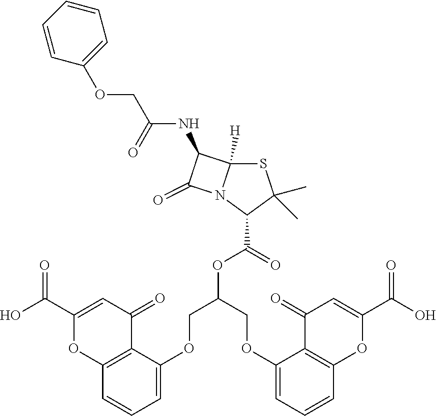 [SMILES: CC1(C)S[C@@H]2[C@H](NC(=O)COc3ccccc3)C(=O)N2[C@H]1C(=O)OC(COc1cccc2oc(C(=O)O)cc(=O)c12)COc1cccc2oc(C(=O)O)cc(=O)c12] |
| 17 | 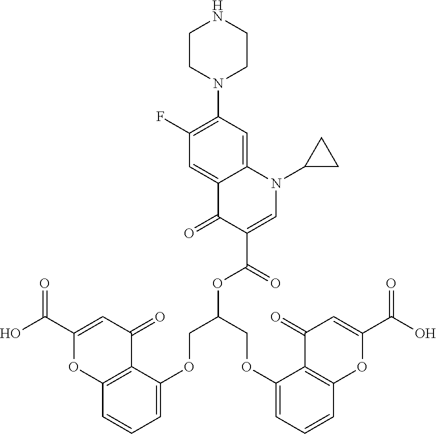 [SMILES: O=C(O)c1cc(=O)c2c(OCC(COc3cccc4oc(C(=O)O)cc(=O)c34)OC(=O)c3cn(C4CC4)c4cc(N5CCNCC5)c(F)cc4c3=O)cccc2o1] |
| 18 | 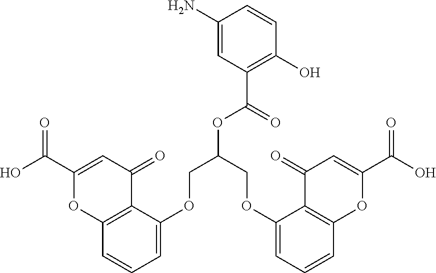 [SMILES: Nc1ccc(O)c(C(=O)OC(COc2cccc3oc(C(=O)O)cc(=O)c23)COc2cccc3oc(C(=O)O)cc(=O)c23)c1] |
| 19 | 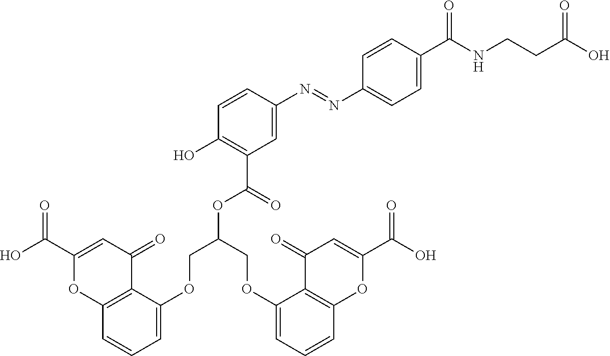 [SMILES: O=C(O)CCNC(=O)c1ccc(/N=N/c2ccc(O)c(C(=O)OC(COc3cccc4oc(C(=O)O)cc(=O)c34)COc3cccc4oc(C(=O)O)cc(=O)c34)c2)cc1] |
| 20 | 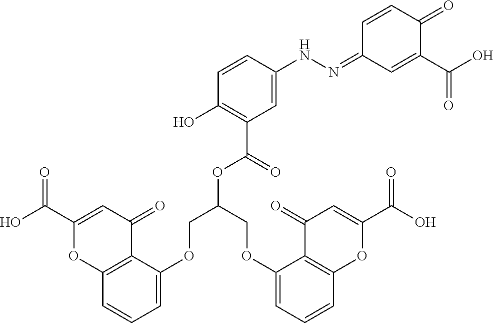 [SMILES: O=C(O)C1=C/C(=N/Nc2ccc(O)c(C(=O)OC(COc3cccc4oc(C(=O)O)cc(=O)c34)COc3cccc4oc(C(=O)O)cc(=O)c34)c2)C=CC1=O] |
| 21 | 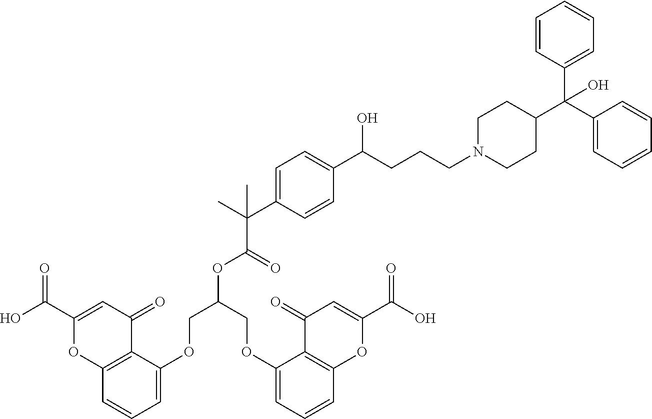 [SMILES: CC(C)(C(=O)OC(COc1cccc2oc(C(=O)O)cc(=O)c12)COc1cccc2oc(C(=O)O)cc(=O)c12)c1ccc(C(O)CCCN2CCC(C(O)(c3ccccc3)c3ccccc3)CC2)cc1] |
| 22 | 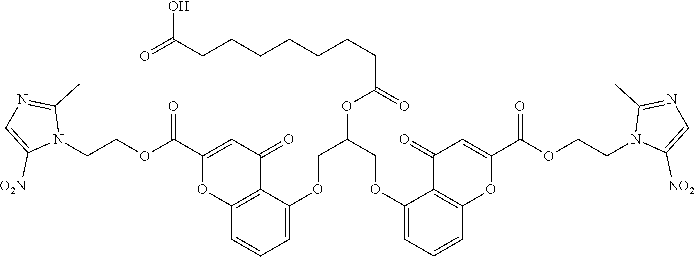 [SMILES: Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)CCCCCCCC(=O)O)cccc2o1] |
| 23 | 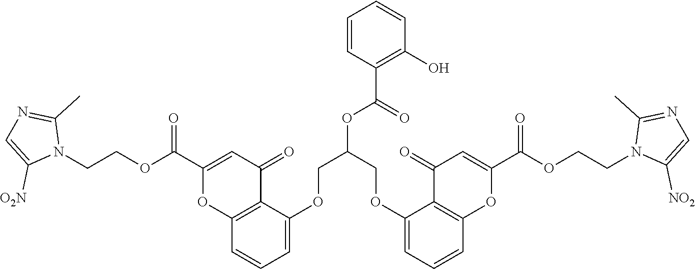 [SMILES: Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)c3ccccc3O)cccc2o1] |
| 24 | 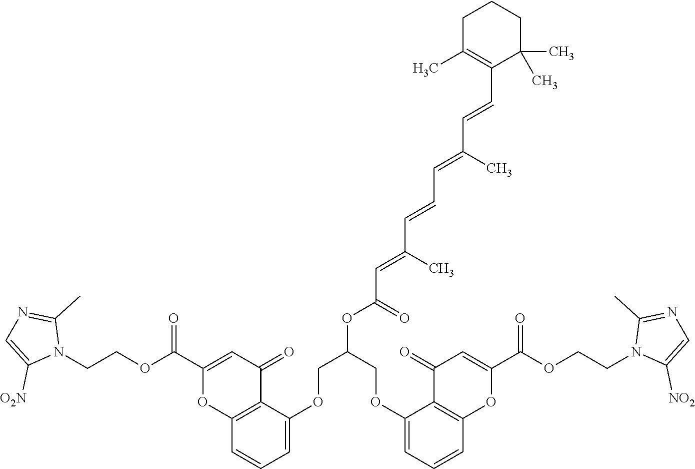 [SMILES: CC1=C(/C=C/C(C)=C/C=C/C(C)=C/C(=O)OC(COc2cccc3oc(C(=O)OCCn4c([N+](=O)[O-])cnc4C)cc(=O)c23)COc2cccc3oc(C(=O)OCCn4c([N+](=O)[O-])cnc4C)cc(=O)c23)C(C)(C)CCC1] |
| 25 | 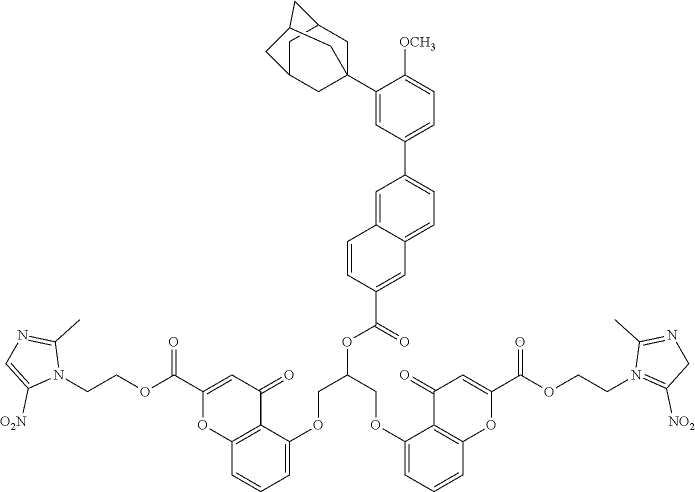 |
| 26 | 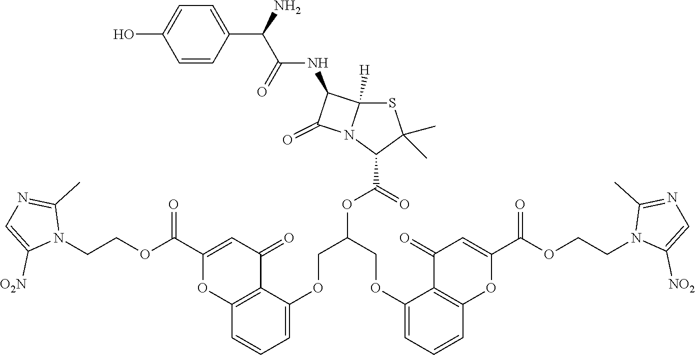 [SMILES: Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)[C@@H]3N4C(=O)[C@@H](NC(=O)[C@H](N)c5ccc(O)cc5)[C@H]4SC3(C)C)cccc2o1] |
| 27 | 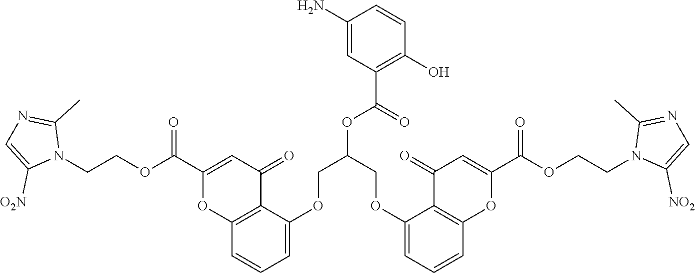 [SMILES: Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)c3cc(N)ccc3O)cccc2o1] |
| 28 | 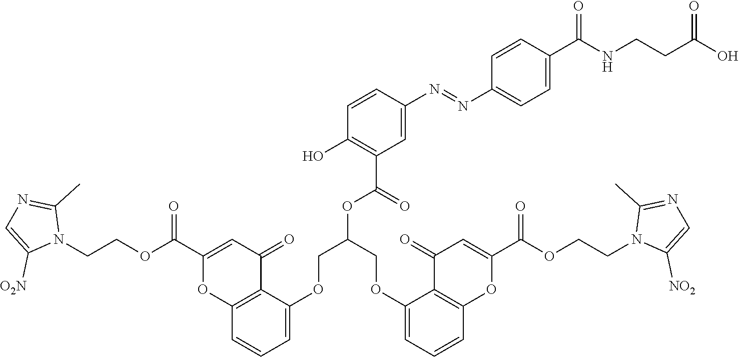 [SMILES: Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)c3cc(/N=N/c4ccc(C(=O)NCCC(=O)O)cc4)ccc3O)cccc2o1] |
| 29 | 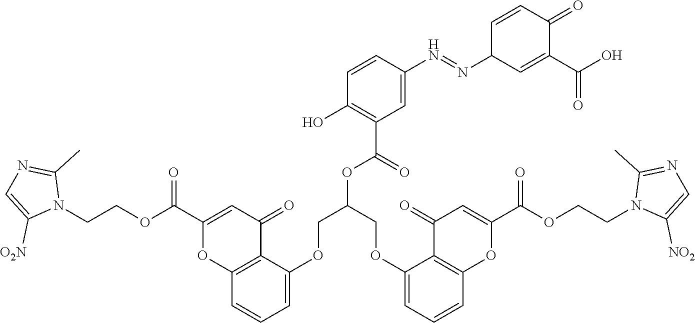 |
| 30 | 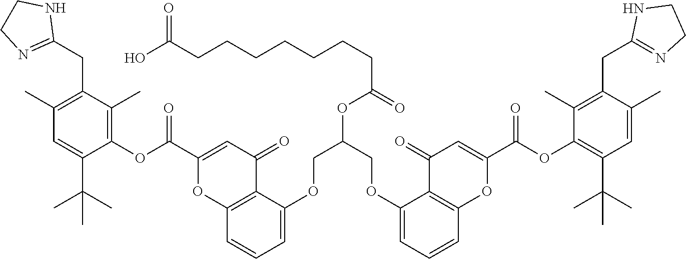 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)CCCCCCCC(=O)O)cccc3o2)c(C)c1CC1=NCCN1] |
| 31 | 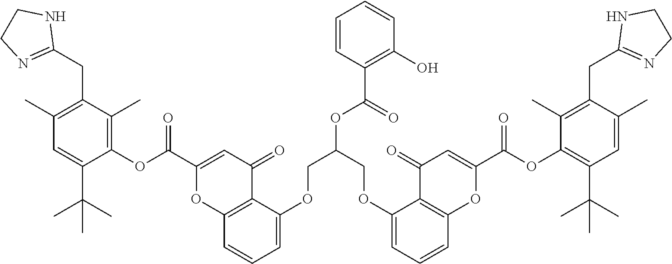 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)c4ccccc4O)cccc3o2)c(C)c1CC1=NCCN1] |
| 32 | 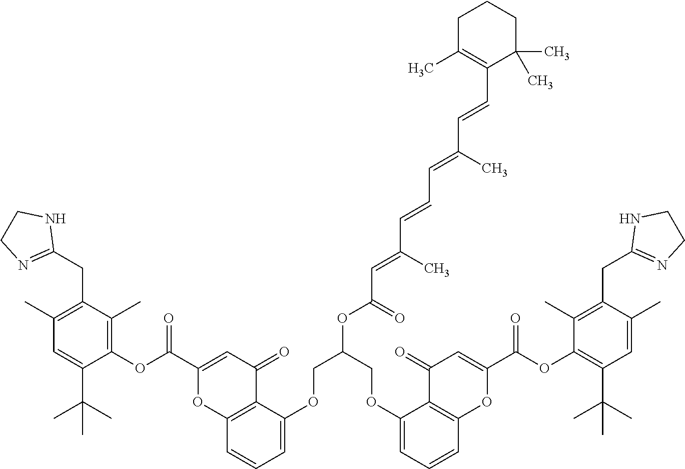 [SMILES: CC1=C(/C=C/C(C)=C/C=C/C(C)=C/C(=O)OC(COc2cccc3oc(C(=O)Oc4c(C(C)(C)C)cc(C)c(CC5=NCCN5)c4C)cc(=O)c23)COc2cccc3oc(C(=O)Oc4c(C(C)(C)C)cc(C)c(CC5=NCCN5)c4C)cc(=O)c23)C(C)(C)CCC1] |
| 33 | 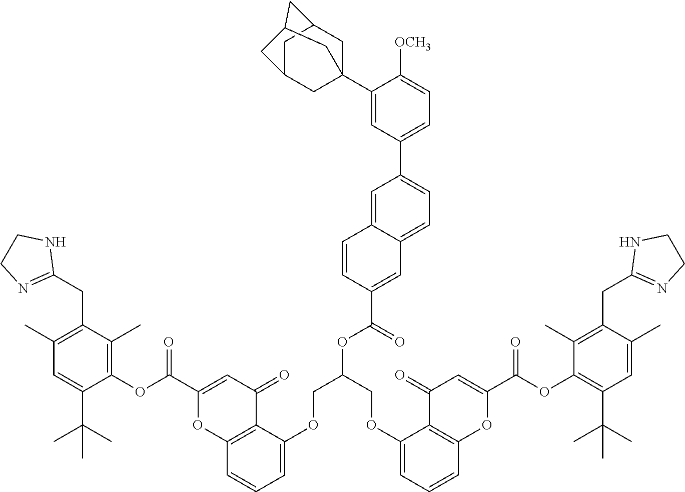 [SMILES: COc1ccc(-c2ccc3cc(C(=O)OC(COc4cccc5oc(C(=O)Oc6c(C(C)(C)C)cc(C)c(CC7=NCCN7)c6C)cc(=O)c45)COc4cccc5oc(C(=O)Oc6c(C(C)(C)C)cc(C)c(CC7=NCCN7)c6C)cc(=O)c45)ccc3c2)cc1C12CC3CC(CC(C3)C1)C2] |
| 34 | 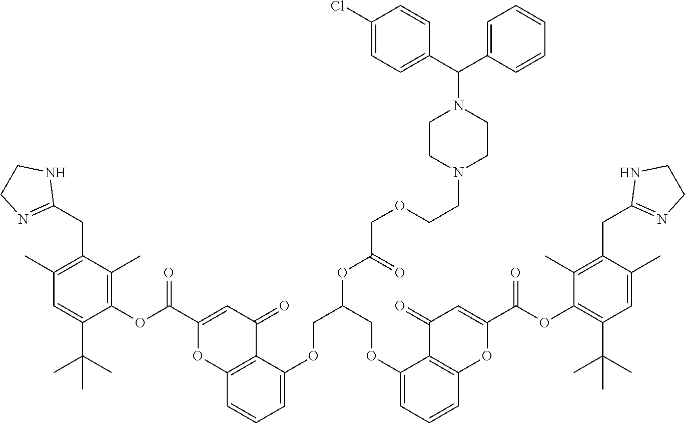 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)COCCN4CCN(C(c5ccccc5)c5ccc(Cl)cc5)CC4)cccc3o2)c(C)c1CC1=NCCN1] |
| 35 | 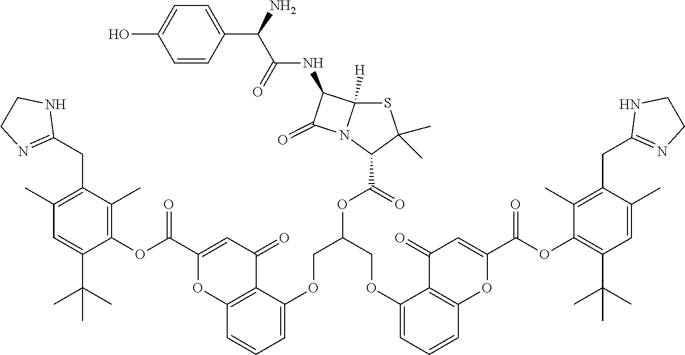 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)[C@@H]4N5C(=O)[C@@H](NC(=O)[C@H](N)c6ccc(O)cc6)[C@H]5SC4(C)C)cccc3o2)c(C)c1CC1=NCCN1] |
| 36 | 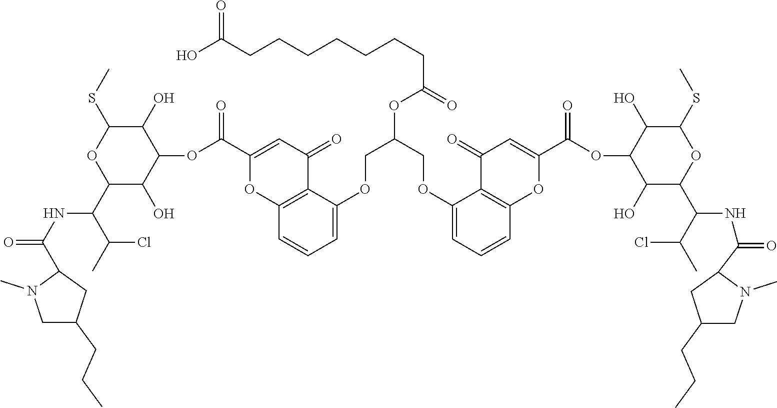 |
| 37 | 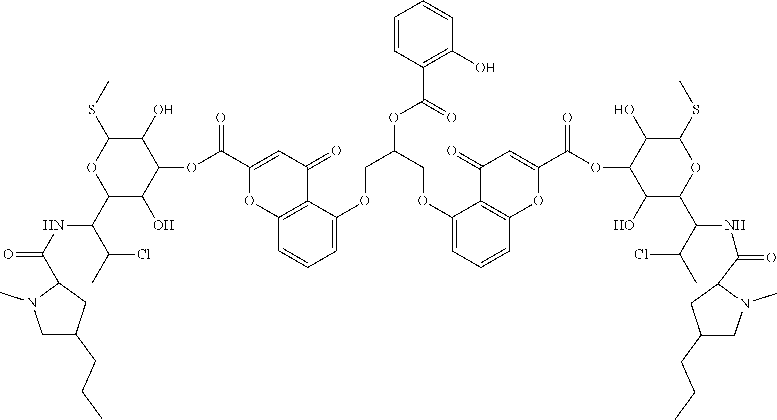 |
| 38 | 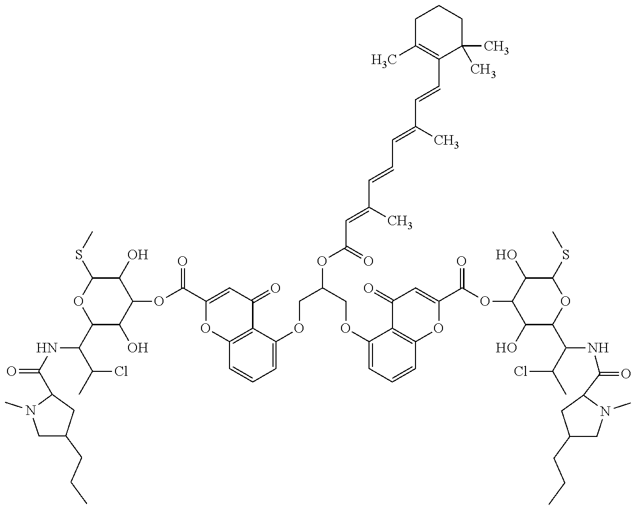 |
| 39 | 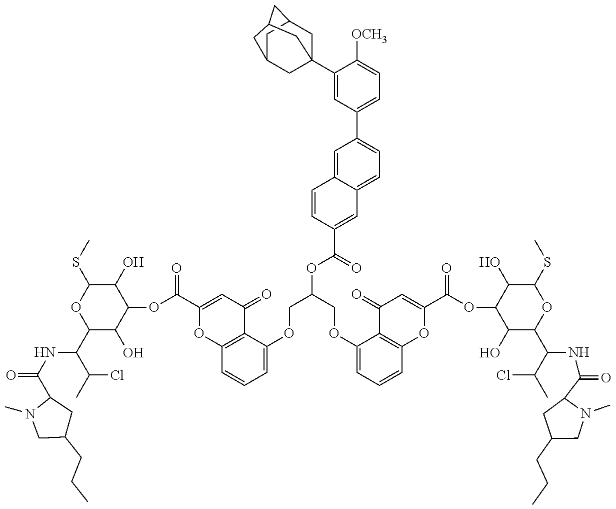 |
| 40 | 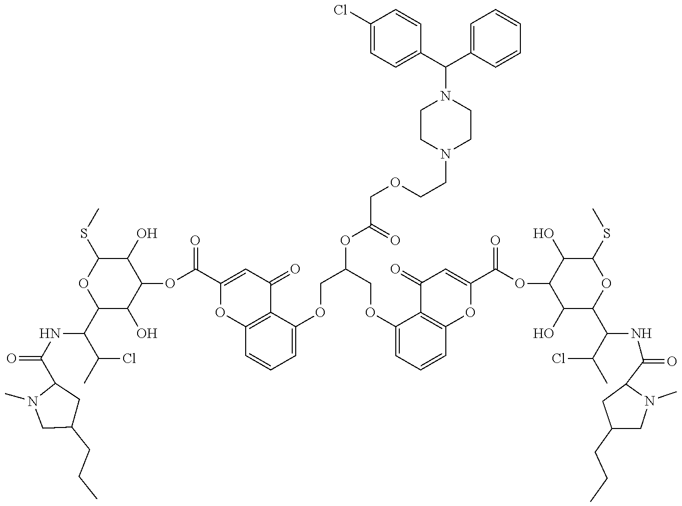 |
| 41 | 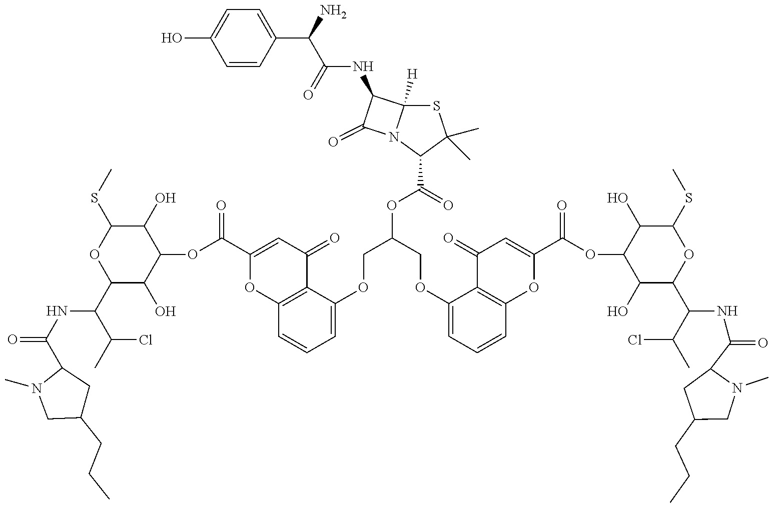 |
| 42 | 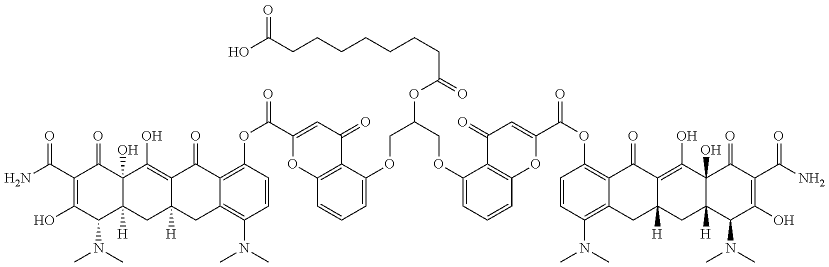 [SMILES: CN(C)c1ccc(OC(=O)c2cc(=O)c3c(OCC(COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)OC(=O)CCCCCCCC(=O)O)cccc3o2)c2c1C[C@H]1C[C@H]3[C@H](N(C)C)C(O)=C(C(N)=O)C(=O)[C@@]3(O)C(O)=C1C2=O] |
| 43 | 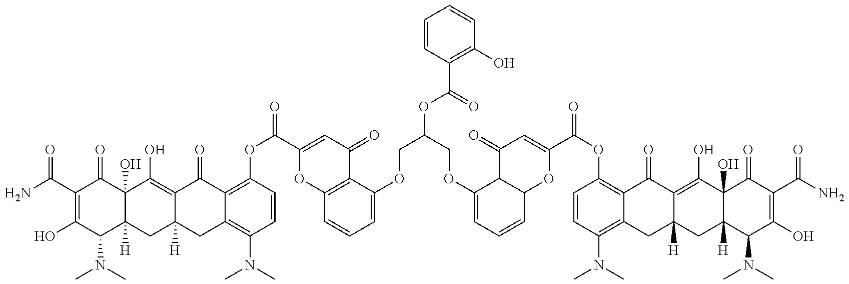 [SMILES: CN(C)c1ccc(OC(=O)C2=CC(=O)C3C(OCC(COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)OC(=O)c4ccccc4O)=CC=CC3O2)c2c1C[C@H]1C[C@H]3[C@H](N(C)C)C(O)=C(C(N)=O)C(=O)[C@@]3(O)C(O)=C1C2=O] |
| 44 | 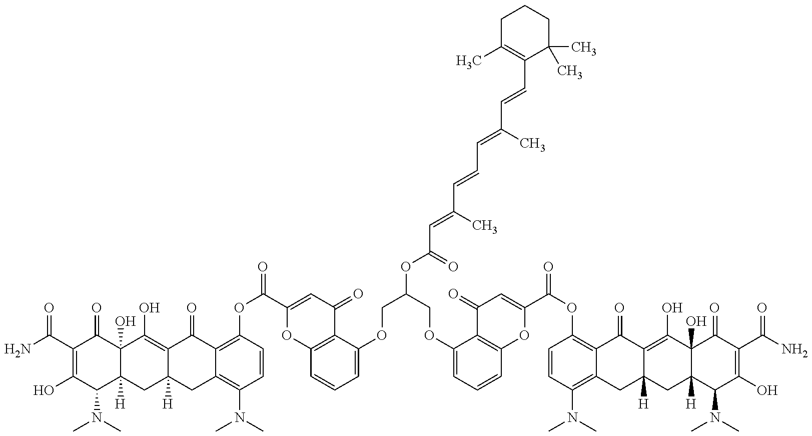 [SMILES: CC1=C(/C=C/C(C)=C/C=C/C(C)=C/C(=O)OC(COc2cccc3oc(C(=O)Oc4ccc(N(C)C)c5c4C(=O)C4=C(O)[C@]6(O)C(=O)C(C(N)=O)=C(O)[C@@H](N(C)C)[C@@H]6C[C@@H]4C5)cc(=O)c23)COc2cccc3oc(C(=O)Oc4ccc(N(C)C)c5c4C(=O)C4=C(O)[C@]6(O)C(=O)C(C(N)=O)=C(O)[C@@H](N(C)C)[C@@H]6C[C@@H]4C5)cc(=O)c23)C(C)(C)CCC1] |
| 45 | 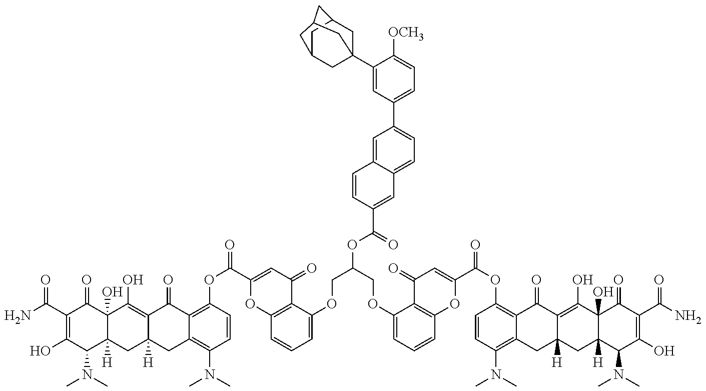 [SMILES: COc1ccc(-c2ccc3cc(C(=O)OC(COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)ccc3c2)cc1C12CC3CC(CC(C3)C1)C2] |
| 46 | 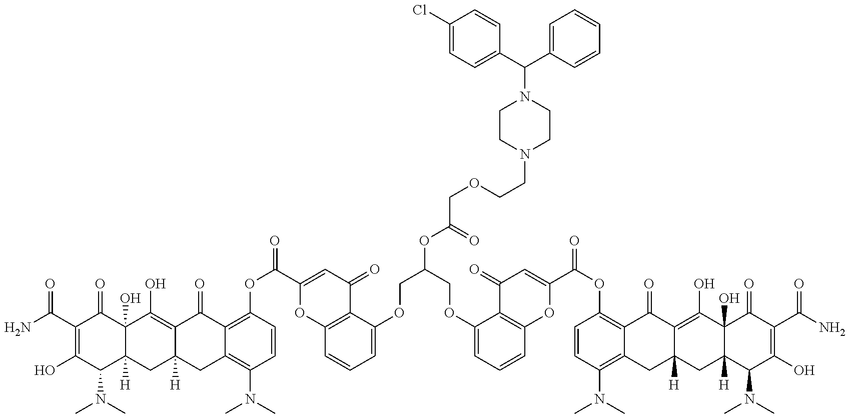 [SMILES: CN(C)c1ccc(OC(=O)c2cc(=O)c3c(OCC(COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)OC(=O)COCCN4CCN(C(c5ccccc5)c5ccc(Cl)cc5)CC4)cccc3o2)c2c1C[C@H]1C[C@H]3[C@H](N(C)C)C(O)=C(C(N)=O)C(=O)[C@@]3(O)C(O)=C1C2=O] |
| 47 | 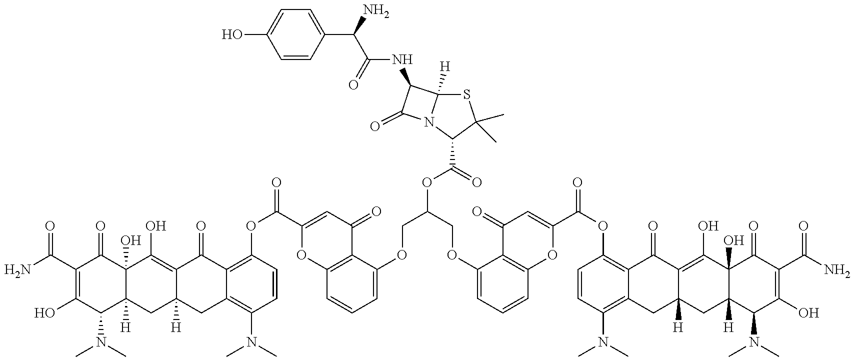 [SMILES: CN(C)c1ccc(OC(=O)c2cc(=O)c3c(OCC(COc4cccc5oc(C(=O)Oc6ccc(N(C)C)c7c6C(=O)C6=C(O)[C@]8(O)C(=O)C(C(N)=O)=C(O)[C@@H](N(C)C)[C@@H]8C[C@@H]6C7)cc(=O)c45)OC(=O)[C@@H]4N5C(=O)[C@@H](NC(=O)[C@H](N)c6ccc(O)cc6)[C@H]5SC4(C)C)cccc3o2)c2c1C[C@H]1C[C@H]3[C@H](N(C)C)C(O)=C(C(N)=O)C(=O)[C@@]3(O)C(O)=C1C2=O] |
| 48 | 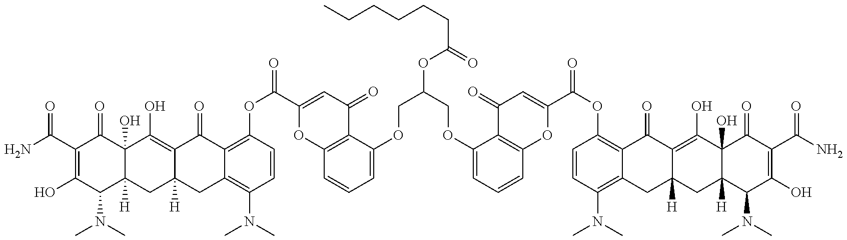 [SMILES: CCCCCCC(=O)OC(COc1cccc2oc(C(=O)Oc3ccc(N(C)C)c4c3C(=O)C3=C(O)[C@]5(O)C(=O)C(C(N)=O)=C(O)[C@@H](N(C)C)[C@@H]5C[C@@H]3C4)cc(=O)c12)COc1cccc2oc(C(=O)Oc3ccc(N(C)C)c4c3C(=O)C3=C(O)[C@]5(O)C(=O)C(C(N)=O)=C(O)[C@@H](N(C)C)[C@@H]5C[C@@H]3C4)cc(=O)c12] |
| 49 | 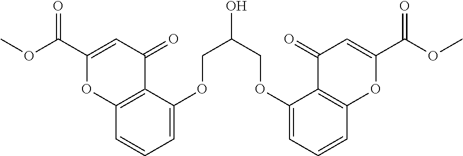 |
| 50 | 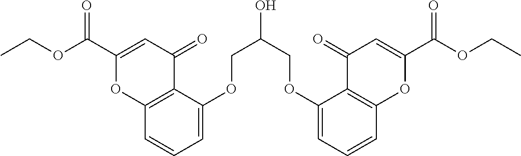 [SMILES: CCOC(=O)c1cc(=O)c2c(OCC(O)COc3cccc4oc(C(=O)OCC)cc(=O)c34)cccc2o1] |
| 51 | 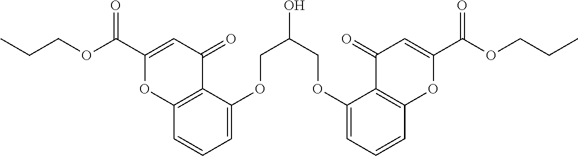 [SMILES: CCCOC(=O)c1cc(=O)c2c(OCC(O)COc3cccc4oc(C(=O)OCCC)cc(=O)c34)cccc2o1] |
| 52 | 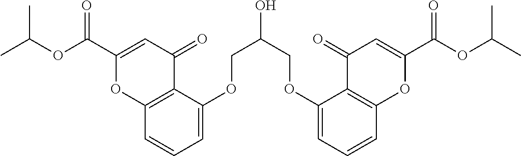 [SMILES: CC(C)OC(=O)c1cc(=O)c2c(OCC(O)COc3cccc4oc(C(=O)OC(C)C)cc(=O)c34)cccc2o1] |
| 53 | 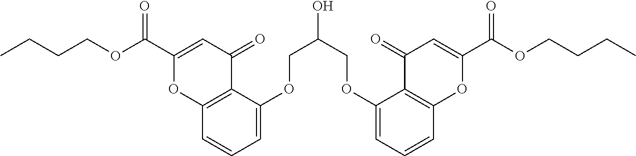 [SMILES: CCCCOC(=O)c1cc(=O)c2c(OCC(O)COc3cccc4oc(C(=O)OCCCC)cc(=O)c34)cccc2o1] |
| 54 | 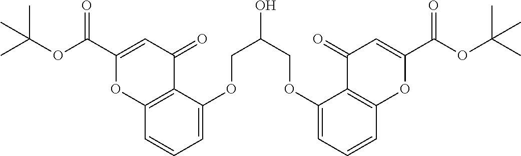 [SMILES: CC(C)(C)OC(=O)c1cc(=O)c2c(OCC(O)COc3cccc4oc(C(=O)OC(C)(C)C)cc(=O)c34)cccc2o1] |
| 55 | 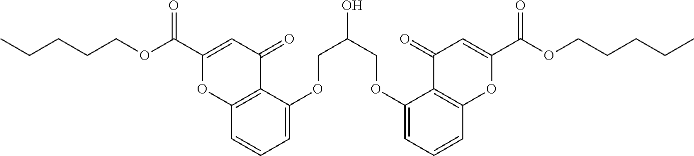 [SMILES: CCCCCOC(=O)c1cc(=O)c2c(OCC(O)COc3cccc4oc(C(=O)OCCCCC)cc(=O)c34)cccc2o1] |
| 56 | 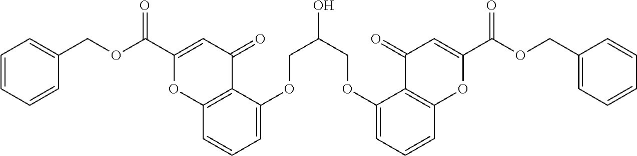 [SMILES: O=C(OCc1ccccc1)c1cc(=O)c2c(OCC(O)COc3cccc4oc(C(=O)OCc5ccccc5)cc(=O)c34)cccc2o1] |
| 57 | 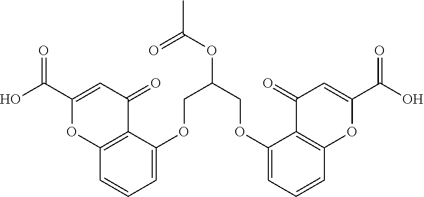 [SMILES: CC(=O)OC(COc1cccc2oc(C(=O)O)cc(=O)c12)COc1cccc2oc(C(=O)O)cc(=O)c12] |
| 58 | 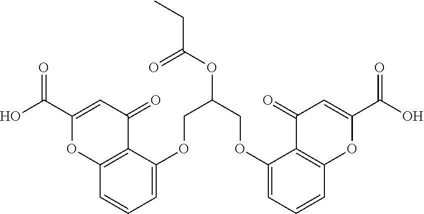 [SMILES: CCC(=O)OC(COc1cccc2oc(C(=O)O)cc(=O)c12)COc1cccc2oc(C(=O)O)cc(=O)c12] |
| 59 | 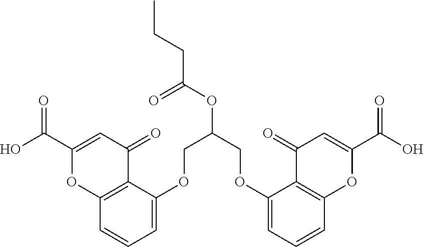 [SMILES: CCCC(=O)OC(COc1cccc2oc(C(=O)O)cc(=O)c12)COc1cccc2oc(C(=O)O)cc(=O)c12] |
| 60 | 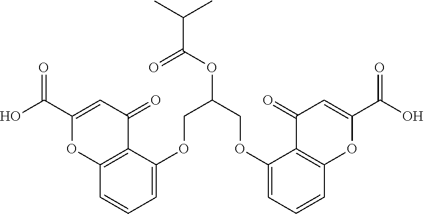 [SMILES: CC(C)C(=O)OC(COc1cccc2oc(C(=O)O)cc(=O)c12)COc1cccc2oc(C(=O)O)cc(=O)c12] |
| 61 | 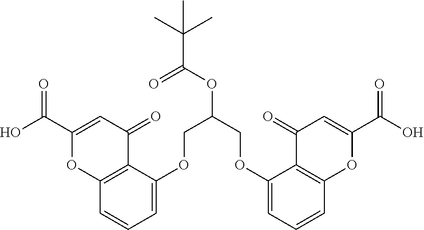 [SMILES: CC(C)(C)C(=O)OC(COc1cccc2oc(C(=O)O)cc(=O)c12)COc1cccc2oc(C(=O)O)cc(=O)c12] |
| 62 | 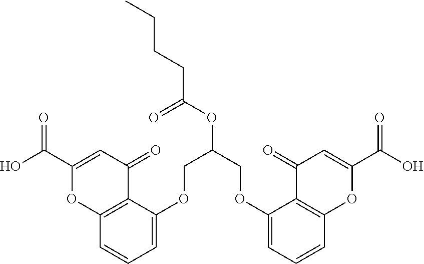 [SMILES: CCCCC(=O)OC(COc1cccc2oc(C(=O)O)cc(=O)c12)COc1cccc2oc(C(=O)O)cc(=O)c12] |
| 63 | 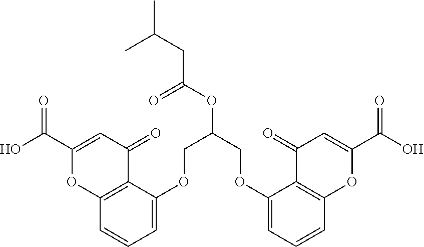 [SMILES: CC(C)CC(=O)OC(COc1cccc2oc(C(=O)O)cc(=O)c12)COc1cccc2oc(C(=O)O)cc(=O)c12] |
| 64 | 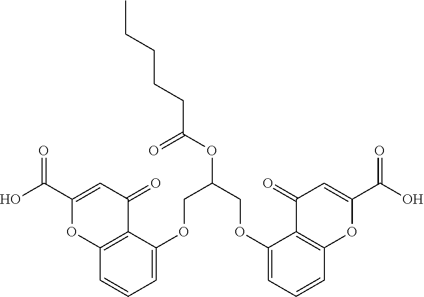 [SMILES: CCCCCC(=O)OC(COc1cccc2oc(C(=O)O)cc(=O)c12)COc1cccc2oc(C(=O)O)cc(=O)c12] |
| 65 | 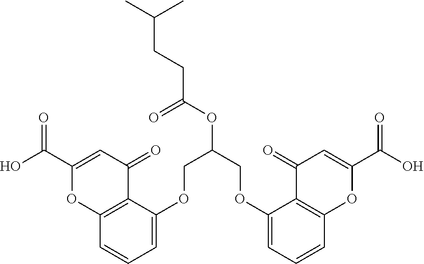 [SMILES: CC(C)CCC(=O)OC(COc1cccc2oc(C(=O)O)cc(=O)c12)COc1cccc2oc(C(=O)O)cc(=O)c12] |
| 66 | 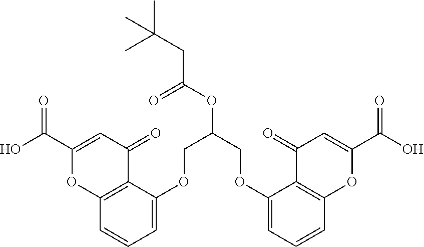 [SMILES: CC(C)(C)CC(=O)OC(COc1cccc2oc(C(=O)O)cc(=O)c12)COc1cccc2oc(C(=O)O)cc(=O)c12] |
| 67 | 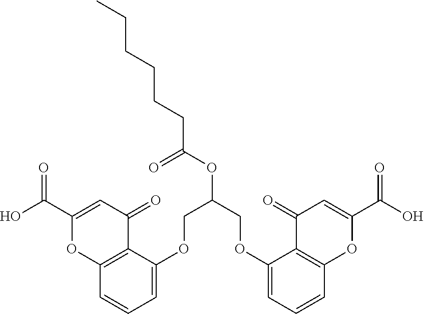 [SMILES: CCCCCCC(=O)OC(COc1cccc2oc(C(=O)O)cc(=O)c12)COc1cccc2oc(C(=O)O)cc(=O)c12] |
| 68 | 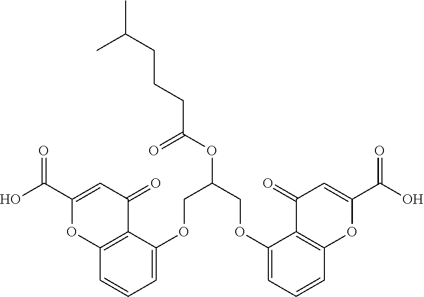 [SMILES: CC(C)CCCC(=O)OC(COc1cccc2oc(C(=O)O)cc(=O)c12)COc1cccc2oc(C(=O)O)cc(=O)c12] |
| 69 | 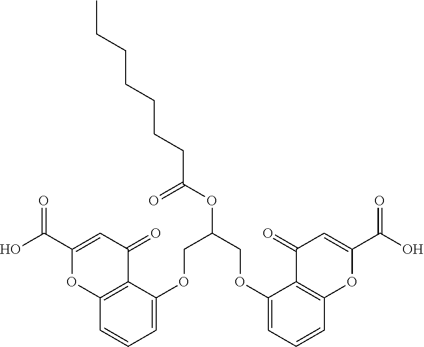 [SMILES: CCCCCCCC(=O)OC(COc1cccc2oc(C(=O)O)cc(=O)c12)COc1cccc2oc(C(=O)O)cc(=O)c12] |
| 70 | 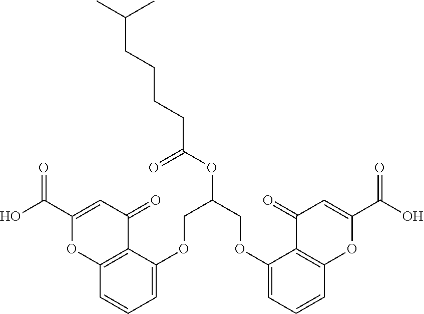 [SMILES: CC(C)CCCCC(=O)OC(COc1cccc2oc(C(=O)O)cc(=O)c12)COc1cccc2oc(C(=O)O)cc(=O)c12] |
| 71 | 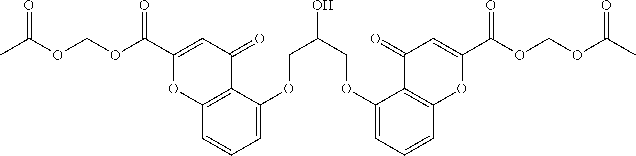 [SMILES: CC(=O)OCOC(=O)c1cc(=O)c2c(OCC(O)COc3cccc4oc(C(=O)OCOC(C)=O)cc(=O)c34)cccc2o1] |
| 72 | 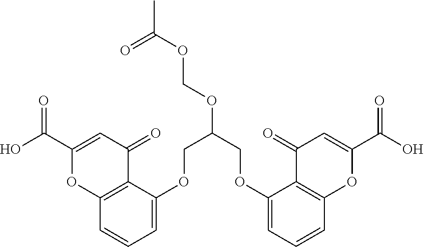 [SMILES: CC(=O)OCOC(COc1cccc2oc(C(=O)O)cc(=O)c12)COc1cccc2oc(C(=O)O)cc(=O)c12] |
| 73 | 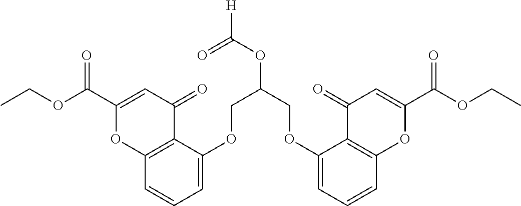 [SMILES: CCOC(=O)c1cc(=O)c2c(OCC(COc3cccc4oc(C(=O)OCC)cc(=O)c34)OC=O)cccc2o1] |
| 74 | 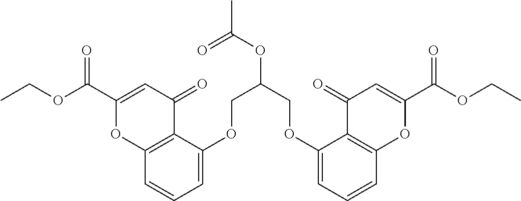 [SMILES: CCOC(=O)c1cc(=O)c2c(OCC(COc3cccc4oc(C(=O)OCC)cc(=O)c34)OC(C)=O)cccc2o1] |
| 75 | 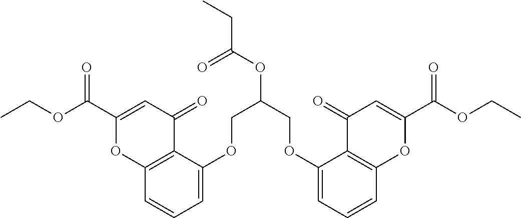 [SMILES: CCOC(=O)c1cc(=O)c2c(OCC(COc3cccc4oc(C(=O)OCC)cc(=O)c34)OC(=O)CC)cccc2o1] |
| 76 | 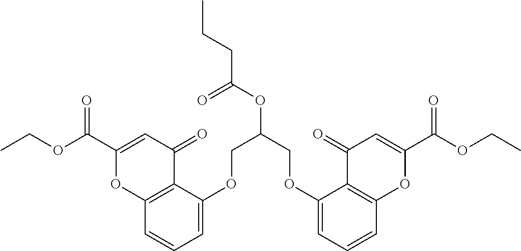 [SMILES: CCCC(=O)OC(COc1cccc2oc(C(=O)OCC)cc(=O)c12)COc1cccc2oc(C(=O)OCC)cc(=O)c12] |
| 77 | 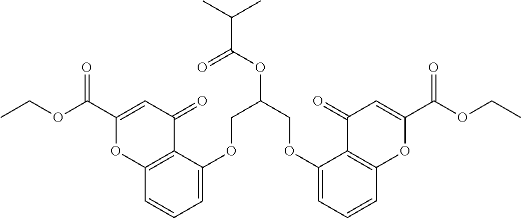 [SMILES: CCOC(=O)c1cc(=O)c2c(OCC(COc3cccc4oc(C(=O)OCC)cc(=O)c34)OC(=O)C(C)C)cccc2o1] |
| 78 | 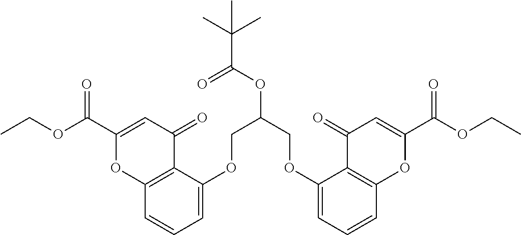 [SMILES: CCOC(=O)c1cc(=O)c2c(OCC(COc3cccc4oc(C(=O)OCC)cc(=O)c34)OC(=O)C(C)(C)C)cccc2o1] |
| 79 | 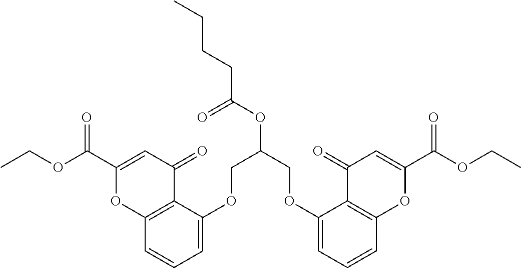 [SMILES: CCCCC(=O)OC(COc1cccc2oc(C(=O)OCC)cc(=O)c12)COc1cccc2oc(C(=O)OCC)cc(=O)c12] |
| 80 | 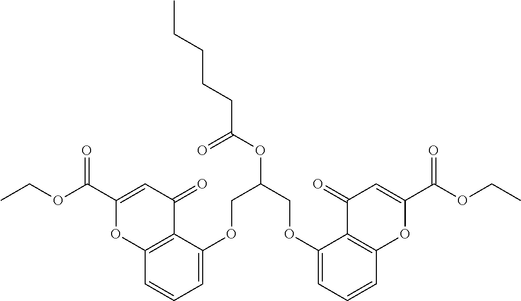 [SMILES: CCCCCC(=O)OC(COc1cccc2oc(C(=O)OCC)cc(=O)c12)COc1cccc2oc(C(=O)OCC)cc(=O)c12] |
| 81 | 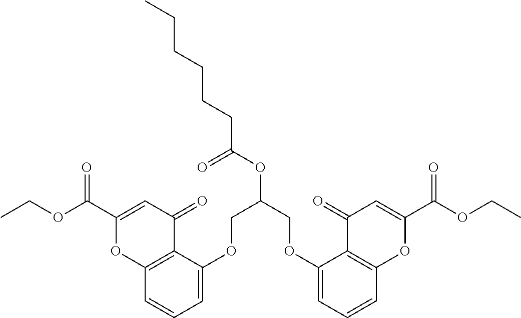 [SMILES: CCCCCCC(=O)OC(COc1cccc2oc(C(=O)OCC)cc(=O)c12)COc1cccc2oc(C(=O)OCC)cc(=O)c12] |
| 82 | 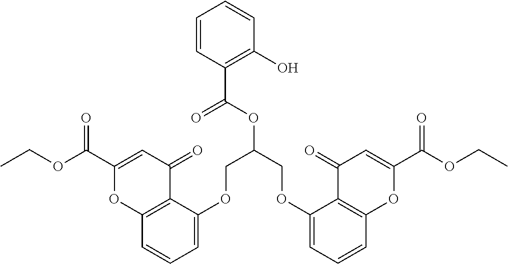 [SMILES: CCOC(=O)c1cc(=O)c2c(OCC(COc3cccc4oc(C(=O)OCC)cc(=O)c34)OC(=O)c3ccccc3O)cccc2o1] |
| 83 | 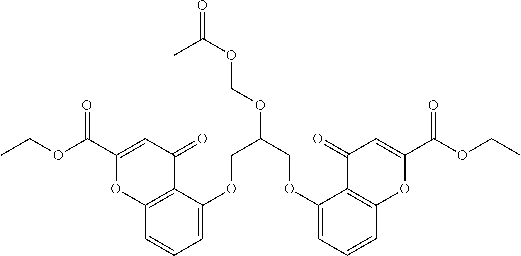 [SMILES: CCOC(=O)c1cc(=O)c2c(OCC(COc3cccc4oc(C(=O)OCC)cc(=O)c34)OCOC(C)=O)cccc2o1] |
| 84 | 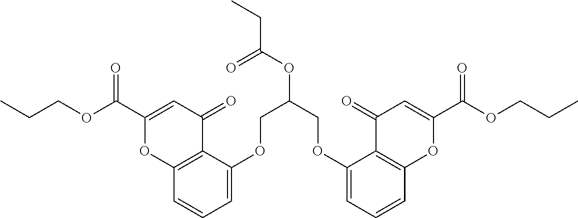 [SMILES: CCCOC(=O)c1cc(=O)c2c(OCC(COc3cccc4oc(C(=O)OCCC)cc(=O)c34)OC(=O)CC)cccc2o1] |

The hydrophobicity of the compounds in Examples 1-48 is illustrated in the following table. Compounds were evaluated using the ACDLabs Freeware software 2021.1.0.

TABLE 2
Hydrophobicity Data.

| | Log P |
|---|---|
| Example | values |
| 1 | 1.26 |
| 2 | 0.46 |
| 3 | 5.27 |
| 4 | 4.24 |
| 5 | 9.42 |
| 6 | 4.04 |
| 7 | −1.08 |
| 8 | 0.36 |
| 9 | 2.52 |
| 10 | 3.92 |
| 11 | 4.20 |
| 12 | 10.44 |
| 13 | 10.31 |
| 14 | 2.36 |
| 15 | 3.09 |
| 16 | 4.02 |
| 17 | 3.51 |
| 18 | 2.53 |
| 19 | 4.84 |
| 20 | 4.44 |
| 21 | 7.39 |
| 22 | 2.89 |
| 23 | 3.17 |
| 24 | 9.41 |
| 25 | 9.28 |
| 26 | 1.32 |
| 27 | 1.50 |
| 28 | 3.81 |
| 29 | 3.41 |
| 30 | 11.04 |
| 31 | 11.33 |
| 32 | 17.56 |
| 33 | 17.43 |
| 34 | 12.39 |
| 35 | 9.48 |
| 36 | 5.67 |
| 37 | 5.95 |
| 38 | 12.19 |
| 39 | 12.06 |
| 40 | 7.02 |
| 41 | 4.10 |
| 42 | 0.54 |
| 43 | 0.82 |
| 44 | 7.06 |
| 45 | 6.88 |
| 46 | 1.89 |
| 47 | −1.02 |
| 48 | 1.58 |
| 49 | 0.47 |
| 50 | 1.53 |
| 51 | 2.59 |
| 52 | 2.23 |
| 53 | 3.66 |
| 54 | 2.92 |
| 55 | 4.72 |
| 56 | 3.53 |
| 57 | 2.30 |
| 58 | 2.96 |
| 59 | 3.37 |
| 60 | 3.18 |
| 61 | 4.06 |
| 62 | 3.90 |
| 63 | 3.71 |
| 64 | 4.43 |
| 65 | 4.24 |
| 66 | 4.06 |
| 67 | 4.96 |
| 68 | 4.78 |
| 69 | 5.49 |
| 70 | 5.31 |
| 71 | 1.01 |
| 72 | 3.42 |
| 73 | 2.10 |
| 74 | 1.54 |
| 75 | 2.07 |
| 76 | 2.6 |
| 77 | 2.42 |
| 78 | 3.3 |
| 79 | 3.13 |
| 80 | 3.66 |
| 81 | 4.19 |
| 82 | 3.44 |
| 83 | 2.66 |
| 84 | 3.13 |

### Pharmacokinetic Studies of Transdermal Administration of the Mutual Prodrug Cetirizine Cromoglicate

Pharmacokinetic (PK) studies are carried out in CD® Hairless rats (Crl: CD-Prss8^{hr}, Charles River Laboratories Inc., Wilmington, MA, USA). The rat model is ideal for dermatology studies and for the safety and efficacy testing of transdermal formulations. The rats are divided into two groups (n=5), one as an intravenous (IV) control and the other as a transdermal delivery group. The animals are anesthetized using ketamine (50 mg/mg) and xylazine (10 mg/kg) via the intraperitoneal route during the study period.

Female CD® Hairless rats weighing 250 to 350 g are housed individually under conditions of controlled temperature (22±2° C.) and relative humidity (45±5%). Animals are fasted overnight before dosing, with free access to water. At least five rats are included for two administration routes: transdermal application and intravenous bolus administration of cetirizine cromoglicate to obtain 100% bioavailability. The experimental animal protocols used in this study are approved by the Institutional Animal Care and Use Committee (IACUC) of Mercer University, according to the National Institutes of Health guidelines (National Institutes of Health Publication Number 85-23, revised 1985) in Principles of Laboratory Animal Care.

To prepare the skin surface for topical cetirizine cromoglicate application, the rat dorsal skin is cleaned using alcohol wipes, and the hair is trimmed 24 h before the application. For transdermal and IV applications, rats are anesthetized by an intraperitoneal injection of a combination of ketamine (50 mg/kg) and xylazine (10 mg/kg).

A cetirizine cromoglicate topical formulation, for example 200 mg/kg, is applied to dorsal rat skin. One can start from basic 50 mg/kg for a first group of animals and double the dosage for a second group of animals (100 mg/kg) and quadruple it (200 mg/kg) for a third group of animals. One group will be receive a placebo only inactive ingredients of topical formulation.

The total application area is 9 cm². The application area of 9 cm² was chosen to obtain plasma concentrations of cetirizine and sodium cromoglicate sufficient for quantification by a validated HPLC-UV method. Upon the induction of anesthesia, the cetirizine cromoglicate topical formulation is applied to the rat dorsal area, followed by a covering of the application area with self-adhesive transparent film Tegaderm™ (3M Health Care, St. Paul, MN) to avoid accidental removal of applied formulation during blood sampling periods.

Whole blood samples are taken during anesthesia and in the conscious state when the rats recover from anesthesia (30-45 min). Blood samples (˜300 μL) are collected from the lateral tail vein and transferred to pre-chilled EDTA-coated tubes at 0.5, 1, 2, 4, 6, 8, 10, and 12 h post-dose. Drug application areas are monitored at each sampling time point for any signs of skin reaction.

At the end of the PK study, the animals are euthanized in a CO₂ chamber, the Tegaderm™ films are removed, and skin areas exposed to topical formulations are collected and snap-frozen using dry ice. Skin tissue samples are analyzed for residues of the mutual prodrug and any hydrolysis products that accumulated in the stratum corneum and viable skin layers epidermis and dermis at the end of the experiment. A benzil (diphenylmethane-1,2-dione) or bis-benzene sulfonamide at a concentration of 50 μM in saline solution is immediately added to the collected skin samples to prevent esterase activity in removed skin. Both compounds have been identified as potent and selective inhibitors of carboxylesterases (CEs) that inhibit carboxylesterases in the low nanomolar range. This inhibition of esterase activity results in a complete halting of carboxylesterase-mediated conversion of the mutual prodrug to its parental prodrugs, allowing for analysis of the ratio of the mutual prodrug to the products of esterase hydrolysis at the end of the transdermal experiment. In addition, determining the total amount of mutual prodrug and parental drugs that still reside in the collected skin allows the correct calculation of the bioavailability of transdermal formulations.

All processed blood and skin tissue samples are stored at −80° C. until further analysis. Blood samples and extracts from skin tissue samples are analyzed using an HPLC-UV method that has been validated for analysis of both cetirizine cromoglicate and its hydrolysis products cetirizine and sodium cromoglicate.

A single dose of cetirizine cromoglicate at 4 mg/kg (2 mg/mL in dimethyl sulfoxide) is administered through lateral tail vein injection following filtration through a 0.22-μm filter. The intravenous doses have been chosen to result in adequate cetirizine and sodium cromoglicate concentrations to quantify and characterize PK. Blood samples are collected from the lateral tail vein pre-dose at 5 min (0.083 h), 0.17, 0.25, 0.5, 1, 2, 4, 8, and 12 h post-dose.

The PK parameters of plasma cetirizine, sodium cromoglicate, and sodium cromoglicate are determined using the noncompartmental analysis (NCA) WinNonlin of Phoenix WinNonlin 8.3 Software (Certara, Inc. Princeton, NJ USA). The Cₘₐₓ and Tₘₐₓ for cetirizine and sodium cromoglicate are determined from the observed values. The terminal elimination rate constant (2z) is estimated by linear regression of the terminal log-linear portion of the plasma concentration-time profiles. The area under the time-concentration curve (AUC) from time zero to the last measurable or sampling time (AUCₗₐₛₜ) is calculated by the logarithmic trapezoidal rule, and the AUC extrapolated to infinity (AUC_{inf}) is obtained as follows:

$${AUC}_{inf} = {AUC}_{last} + \frac{{C}_{last}}{{λ}_{z}}$$

Here, Cₗₐₛₜ is the last sample's concentration. Based on this equation, the AUC_{inf} is extrapolated for a 4 mg/kg intravenous dosage. Transdermal groups using the terminal elimination rate constant (assumed to be universal for all groups) are obtained from the 4 mg/kg intravenous group. The absolute bioavailability (Fab) of the transdermal route is calculated using the following equation, assuming the intravenous 4 mg/kg dose is 100%:

$${F}_{ab} = \frac{{AUC}_{inf , transdermal} \times {Dose}_{iv}}{{AUC}_{inf , iv} \times {Dose}_{transdermal}}$$

The transdermal dose (Dose_{transdermal}) of delivered drugs is an amount of prodrug applied to animal skin after subtraction of an amount of mutual prodrug and parental drugs extracted from skin tissue samples (Dose_{skin residual}) where the transdermal formulation was applied: Dose_{transdermal}=Dose_{applied to the skin}−Dose_{skin residual}). The unit of Dose transdermal is normalized based on the actual average body weight of the rats included. Mutual prodrugs as disclosed herein are expected to be effective in penetrating animal skin and resulting in the sufficient bioavailability and/or dosage of both compounds to support the therapeutic efficacy in the treatment of a skin condition as disclosed herein.

### In-Vitro Membrane Permeation Studies

The vertical PermeGear Franz cell was used to study in vitro transport (diffusion) of cromoglicate sodium and its lipophilic and mutual prodrugs. Shed snakeskin, which is a model membrane to human skin used for preliminary permeability studies due to its similarity in composition to the human stratum corneum, was used as a skin model. The solution in the receiver compartment was maintained at 37° C. and stirred at 500 rpm with a magnetic stirrer. Before each experiment, the shed snakeskin was hydrated in 7.4 pH Dulbecco's Phosphate Buffered Saline (DPBS) containing 0.1% sodium azide solution for 24 hours at room temperature.

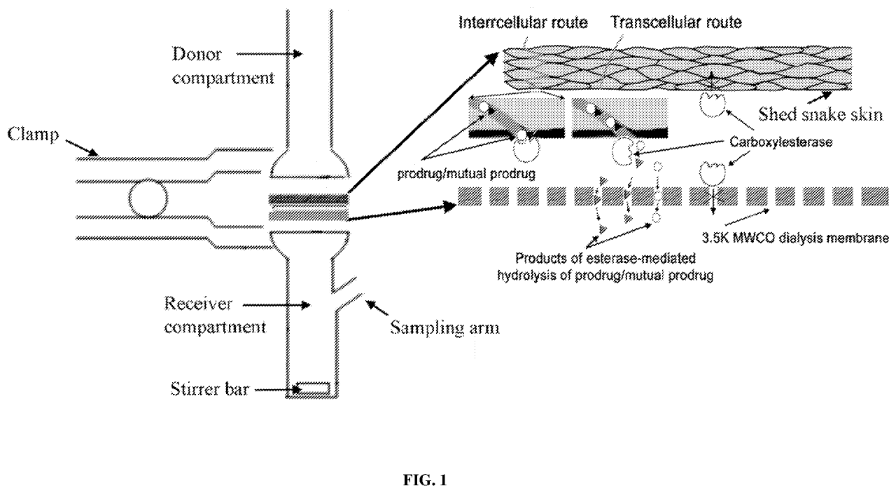

To emulate the esterase activity of human skin, a “sandwich” of shed snakeskin from a black rat snake (Pantherophis obsoletus) and a dialysis membrane with a molecular weight cut-off of 3.5 kDa (Cole-Palmer, Spectra Por S/P 3 EW-02900-04) was designed. Recombinant Human Carboxylesterase 2/CES2 (R&D Systems, #5657-CE) at a concentration 50 μg/mL was added between the snakeskin and the dialysis membrane (FIG. 1). A diffusion area of 0.64 cm² and a volume of 2 mL in the donor and receiver compartments were used. The temperature in Franz cell was controlled via thermocouple probe IT-23 (World Precision Instruments, Sarasota, FL, USA) placed in receiver compartment below the membrane “sandwich” through the sampling arm. All tested compounds were solubilized in in 50% polyethylene glycol 400 (PEG 400)/DPBS at 25 mg/mL. PEG 400 was used to increase the water solubility of diethyl cromoglycate, cetirizine ethyl ester and cetirizine cromoglycate.

The system was allowed to equilibrate for one hour before each experiment. The donor compartment was filled with 2 mL of a single drug suspension (cromoglycate sodium, cetirizine dihydrochloride, prodrugs diethyl cromoglycate and cetirizine ethyl ester or mutual prodrug cetirizine cromoglycate) at a concentration of 25 mg/mL in 50% polyethylene glycol 400/DPBS to ensure constant thermodynamic activity throughout the course of the experiment. The receiver compartment was filled with 2 mL of 50% w/w polyethylene glycol 400 in DPBS to maintain a sink condition in the receiver solution. A sample was manually taken at 1-hour intervals from the receptor compartment and replaced with fresh DPBS at each time point. The concentration of drugs and their cumulative amount was plotted against time. Each experiment with each compound was performed in triplicate. The amount of the drug in the sample was estimated spectrophotometrically (NanoDrop™ UV-Visible spectrophotometer, Thermo Fisher Scientific Inc.). Light absorption was plotted as a function of concentration and fitted with a linear regression equation. The spectrophotometric absorption data for all tested compounds follows a linear regression function (y=mx+b) over a concentration range of 10-60 μg/mL. The concentrations were calculated by using of calibration curves prepared for each individual compound (10 concentrations of standard solutions ranging from 0-60 μg/mL).

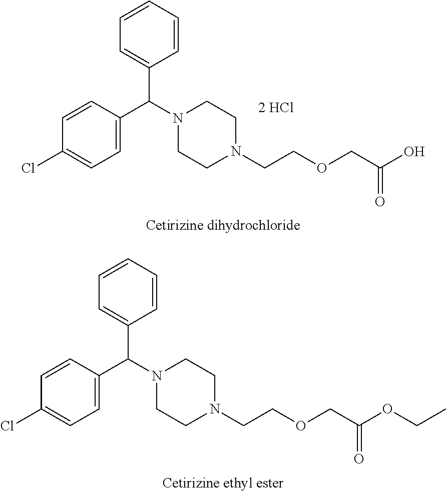 [SMILES: O=C(O)COCCN1CCN(C(c2ccccc2)c2ccc(Cl)cc2)CC1 ; CCOC(=O)COCCN1CCN(C(c2ccccc2)c2ccc(Cl)cc2)CC1]

As shown in Table 3, the permeations of hydrophilic sodium cromoglicate and cetirizine dihydrochloride across the shed snakeskin were 3.5-5 times lower than the permeation of their lipophilic prodrugs diethyl cromoglycate and cetirizine ethyl ester. The transport of the mutual prodrug cetirizine cromoglycate was lower that the permeation of lipophilic prodrugs diethyl cromoglycate and cetirizine ethyl ester, and the mean lag times of cetirizine cromoglycate across shed snakeskin was also longer (0.55-1.15 h) than for cetirizine ethyl ester and diethyl cromoglycate (0.15-0.25 h).

TABLE 3
Membrane Permeation Data

| | Cumulative concentration per unit area (μg/cm²) | | | | |
|---|---|---|---|---|---|
| | | Diethyl | Cetirizine | | |
| | Cetirizine | Cromoglicate | Cromoglicate | Cromoglicate | Cetirizine |
| Time (h) | Ethyl Ester | (Example 50) | (Example 3) | Sodium | Dihydrochloride |
| 0 | 0 | 0 | 0 | 0 | 0 |
| 1 | 54 | 23 | 2 | 5 | 14 |
| 2 | 121 | 55 | 12 | 15 | 22 |
| 3 | 156 | 95 | 28 | 25 | 35 |
| 4 | 212 | 145 | 53 | 30 | 53 |
| 5 | 296 | 195 | 89 | 45 | 73 |
| 6 | 378 | 235 | 112 | 50 | 94 |
| 7 | 468 | 256 | 142 | 73 | 115 |
| 8 | 564 | 320 | 176 | 80 | 130 |
| 9 | 623 | 390 | 215 | 93 | 163 |
| 10 | 695 | 435 | 256 | 98 | 198 |
| 11 | 795 | 470 | 295 | 112 | 215 |
| 12 | 890 | 495 | 350 | 119 | 234 |

While all three compounds have comparable log P values, the molecular weight of cetirizine cromoglycate (MW=839) is 1.6-2 times higher than diethyl cromoglycate (MW=524.5) and cetirizine ethyl ester (MW=417). The absorption of molecules through normal human skin is known to rapidly decline with molecular weights greater than 500 Daltons. Thus, lipophilic molecules like cetirizine cromoglycate can freely diffuse through lipophilic pathways that exist in the lamellar lipid phases. If these molecules do not contain ester bonds they accumulate in the SC, while compounds with ester bonds (such as prodrugs and mutual prodrugs) will be metabolized by epidermis esterases into hydrophilic compounds with lower molecular weights. As such, the hydrolysis of ester bonds in prodrugs and mutual prodrugs facilitates the transport of products of hydrolysis into the viable epidermis.

All references, patents, or applications, US or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

1. A compound of Formula A: 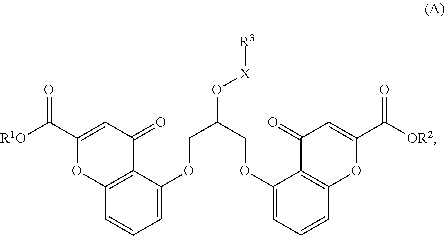
or a salt thereof, wherein:
X is chosen from a bond, —C(O), and -L-C(O)—;
L is chosen from a bond and a linker;
R¹, R², and R³ are independently chosen from hydrogen or alkyl, cycloalkyl, heterocycloalkyl, alkenyl, acetoxy, aryl, heteroaryl, 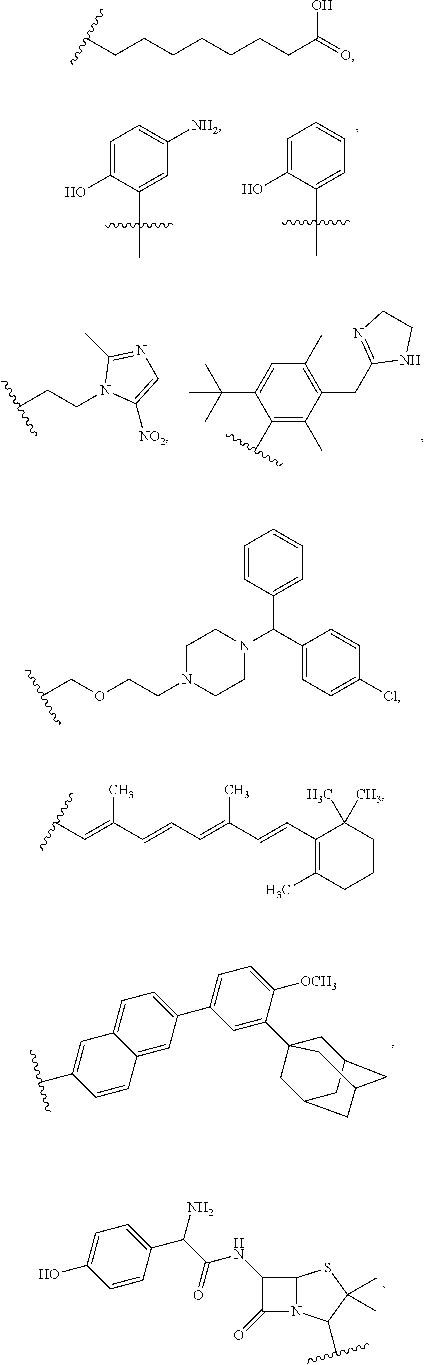 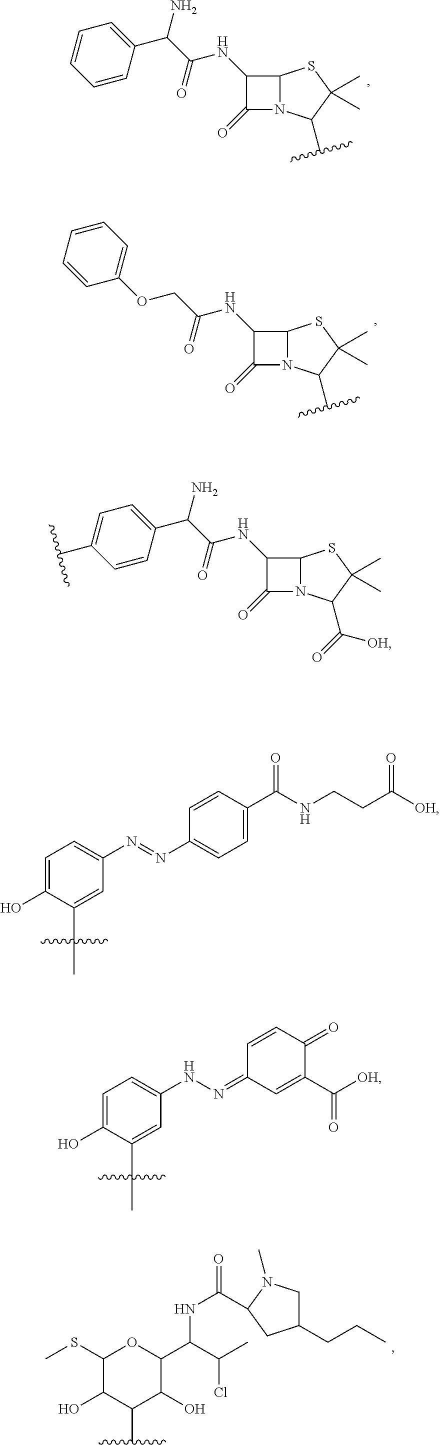 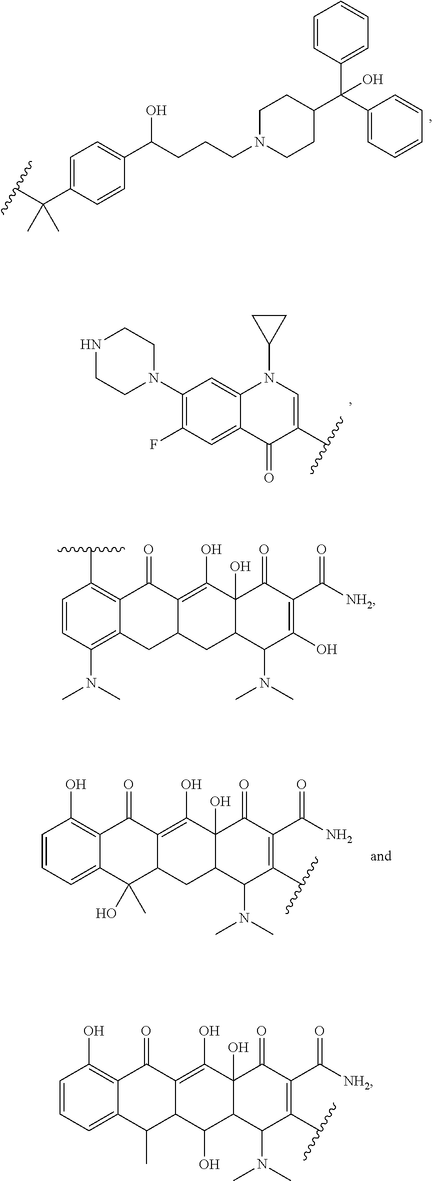 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl; and
at least one of the R¹, R², and R³ is chosen from

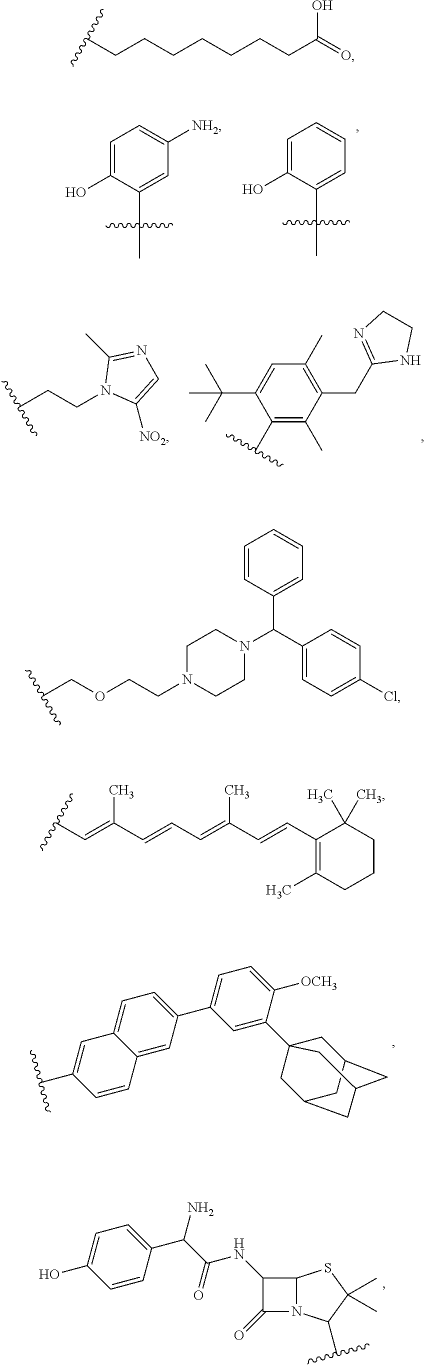

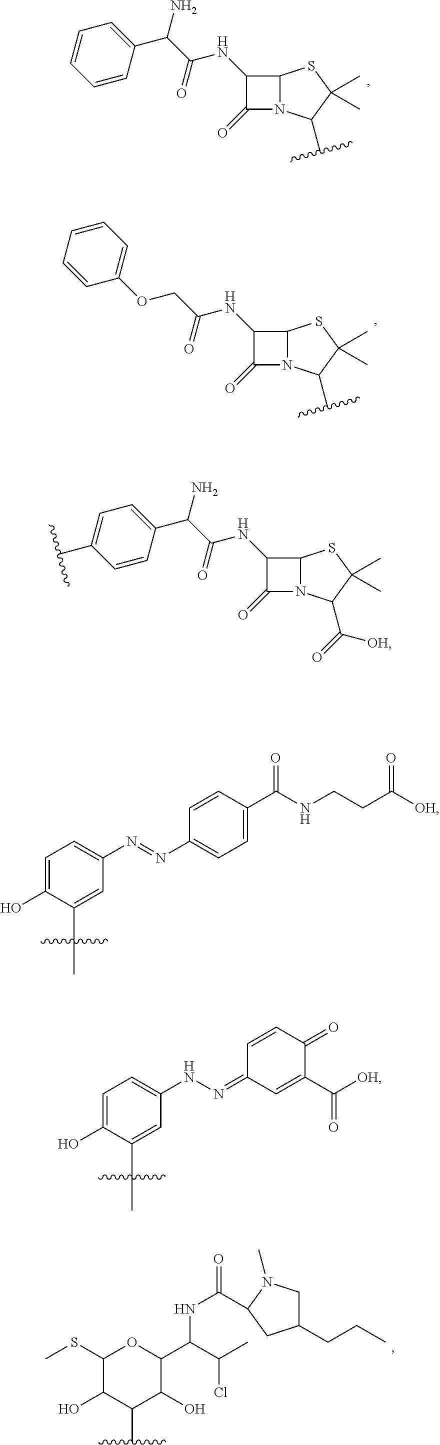

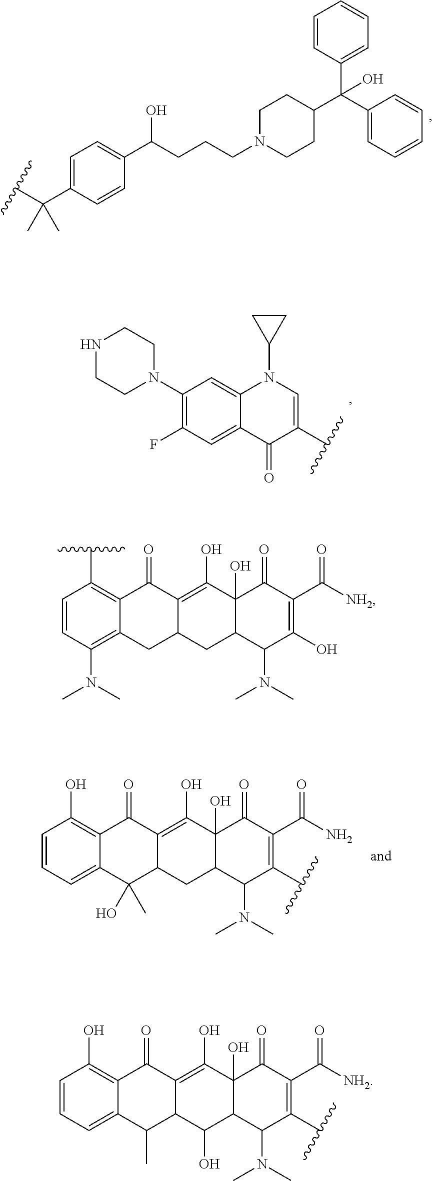 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O ; *c1ccc(N(C)C)c2c1C(=O)C1=C(O)C3(O)C(=O)C(C(N)=O)=C(O)C(N(C)C)C3CC1C2 ; *C1=C(C(N)=O)C(=O)C2(O)C(O)=C3C(=O)c4c(O)cccc4C(C)(O)C3CC2C1N(C)C ; *C1=C(C(N)=O)C(=O)C2(O)C(O)=C3C(=O)c4c(O)cccc4C(C)C3C(O)C2C1N(C)C]

2. The compound of claim 1, wherein the compound is a compound of Formula I: 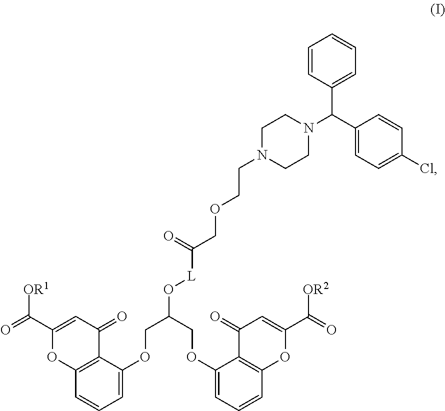
or a salt thereof, wherein:
L is chosen from a bond and a linker; and
R¹ and R² are independently chosen from hydrogen or alkyl, cycloalkyl, heterocycloalkyl, alkenyl, acetoxy, aryl, heteroaryl, 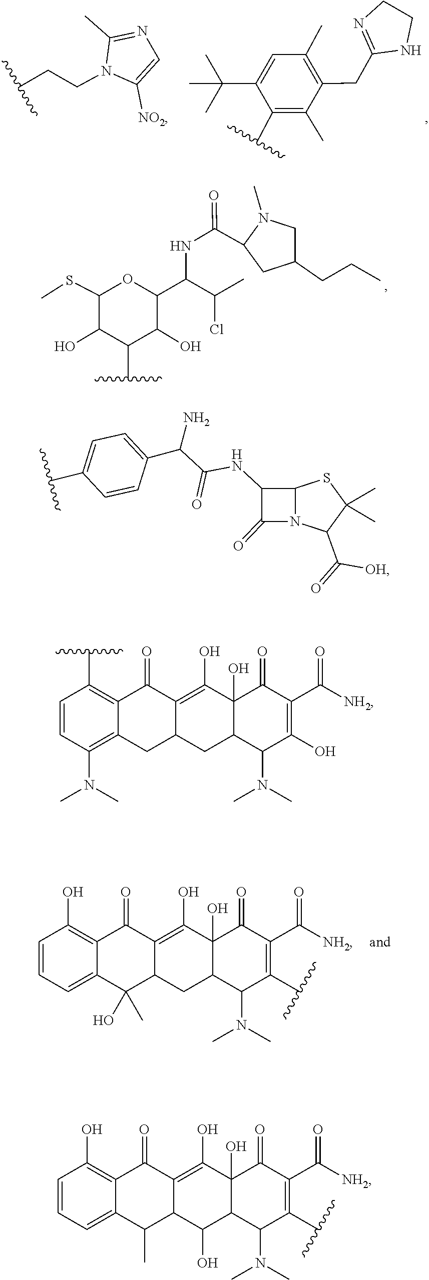
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

3. The compound of claim 1, or a salt thereof, wherein L is a bond.

4. The compound of claim 1, or a salt thereof, wherein L is a linker.

5. The compound of claim 4, or a salt thereof, wherein L is chosen from —C(O)—(CH₂)ₙ—O—, and —C(O)—(CH₂CH₂O)ₙ—, wherein n is an integer chosen from 1 to 12.

6-10. (canceled)

11. The compound of claim 1, or a salt thereof, wherein R¹ and R² are chosen from

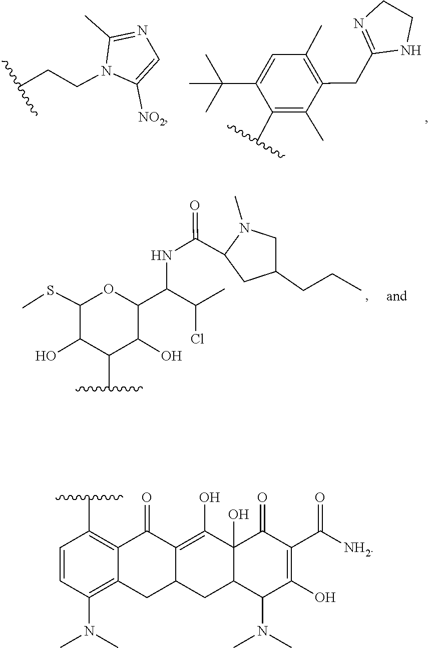

12. The compound of claim 2, wherein the compound of Formula I is chosen from: 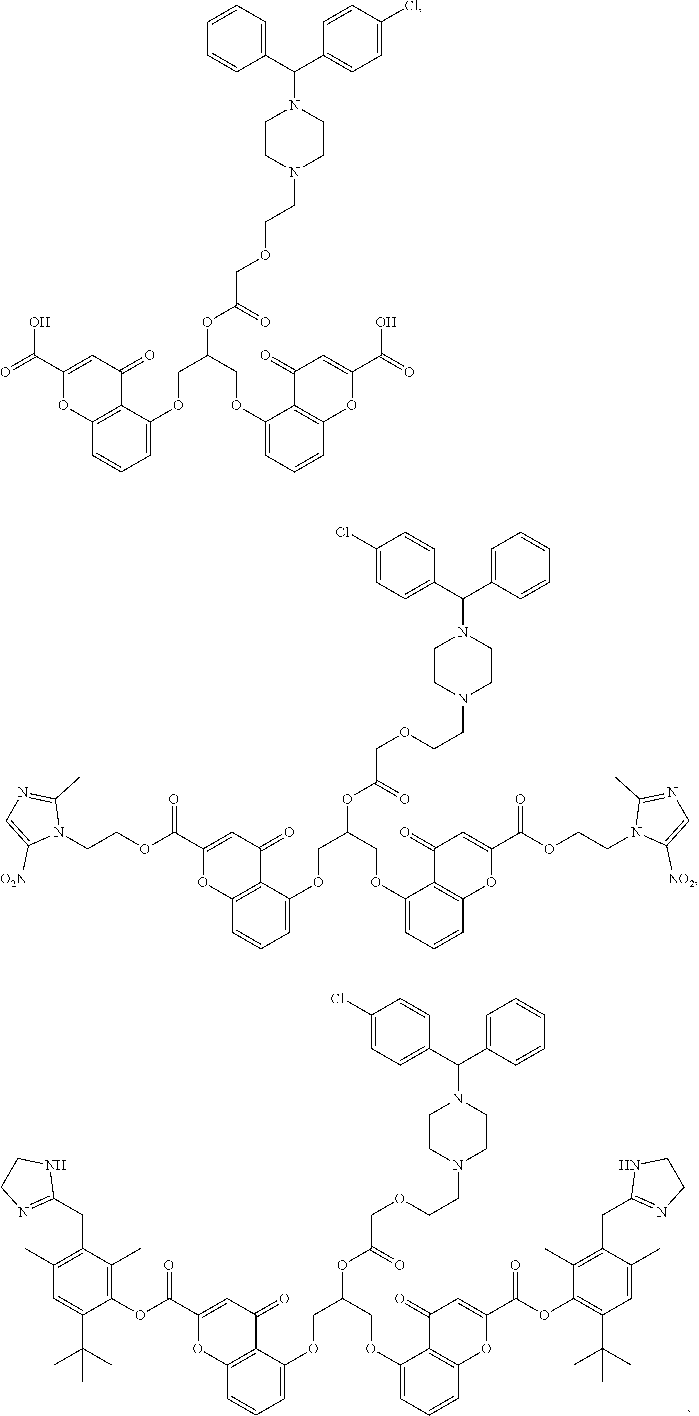 [SMILES: O=C(COCCN1CCN(C(c2ccccc2)c2ccc(Cl)cc2)CC1)OC(COc1cccc2oc(C(=O)O)cc(=O)c12)COc1cccc2oc(C(=O)O)cc(=O)c12 ; Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)COCCN3CCN(C(c4ccccc4)c4ccc(Cl)cc4)CC3)cccc2o1 ; Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)COCCN4CCN(C(c5ccccc5)c5ccc(Cl)cc5)CC4)cccc3o2)c(C)c1CC1=NCCN1] 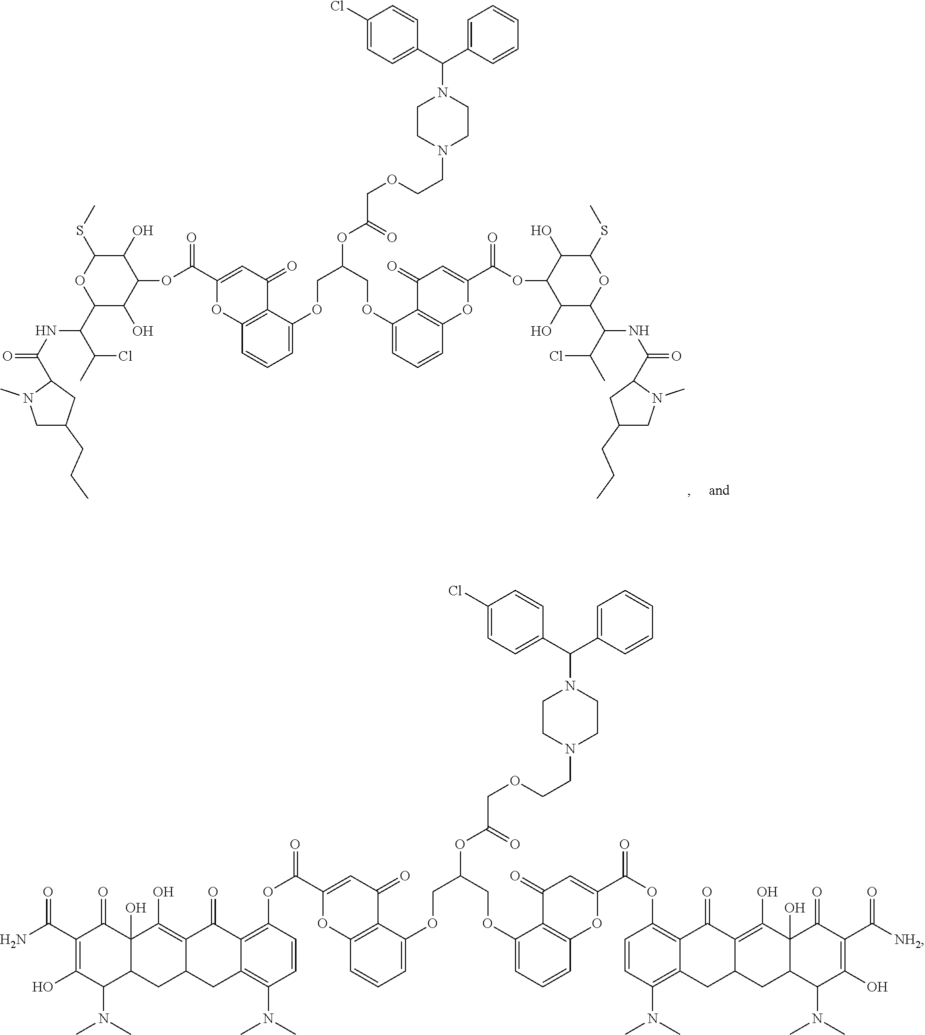
or a salt thereof.

13. The compound of claim 1, wherein the compound is a compound of Formula II: 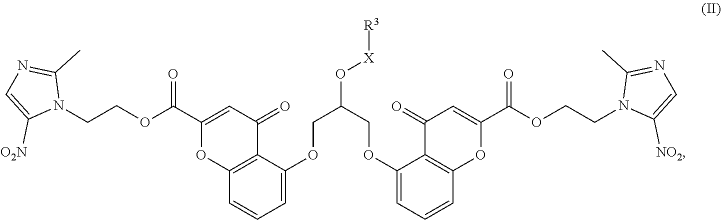
or a salt thereof, wherein:
X is —C(O)— or a bond; and
R³ is hydrogen or is chosen from alkyl, cycloalkyl, heterocycloalkyl, alkenyl, alkoxy, aryl, heteroaryl, 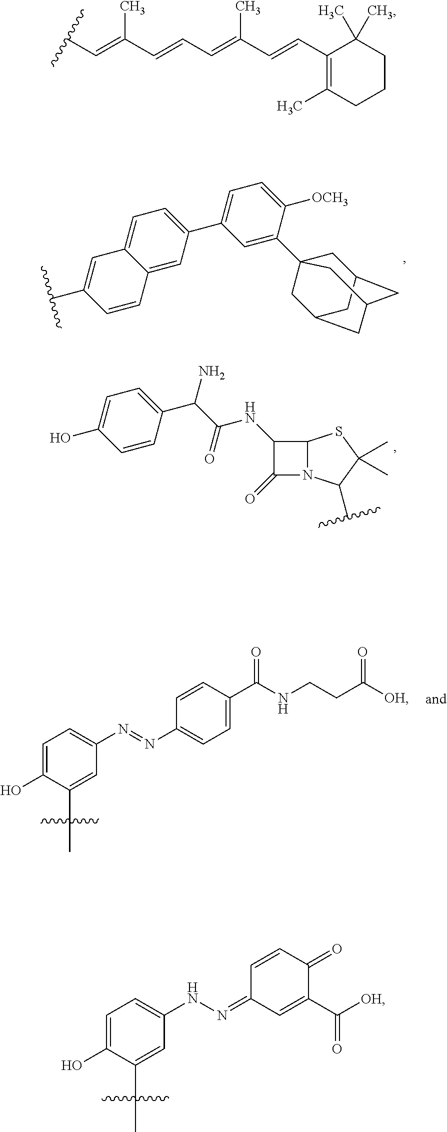 [SMILES: */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *c1cc(/N=N/c2ccc(C(=O)NCCC(=O)O)cc2)ccc1O ; *c1cc(N/N=C2\C=CC(=O)C(C(=O)O)=C2)ccc1O]
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

14. The compound of claim 1, wherein the compound is a compound of Formula III: 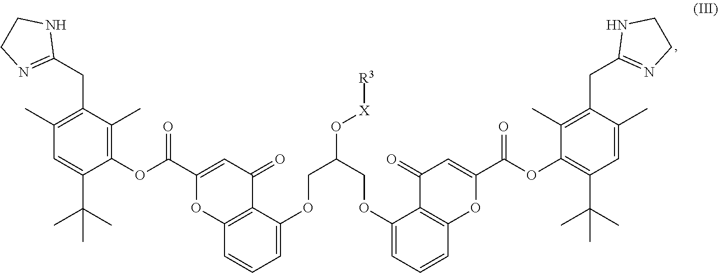
or a salt thereof, wherein:
X is —C(O)— or a bond; and
R³ is hydrogen or is chosen from alkyl, cycloalkyl, heterocycloalkyl, alkenyl, alkoxy, aryl, heteroaryl, 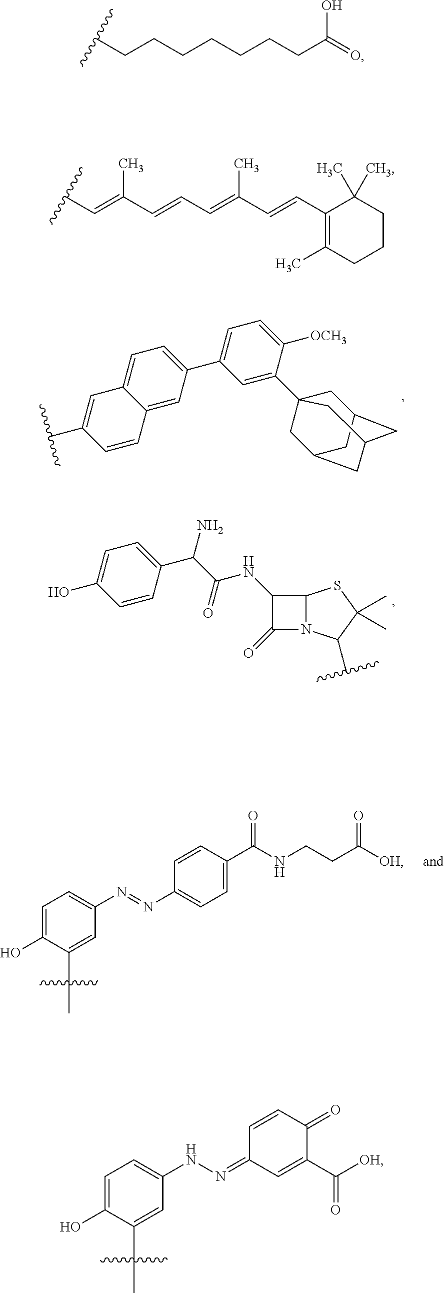 [SMILES: *CCCCCCCC(=O)O ; */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *c1cc(/N=N/c2ccc(C(=O)NCCC(=O)O)cc2)ccc1O ; *c1cc(N/N=C2\C=CC(=O)C(C(=O)O)=C2)ccc1O]
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

15. The compound of claim 1, wherein the compound is a compound of Formula IV: 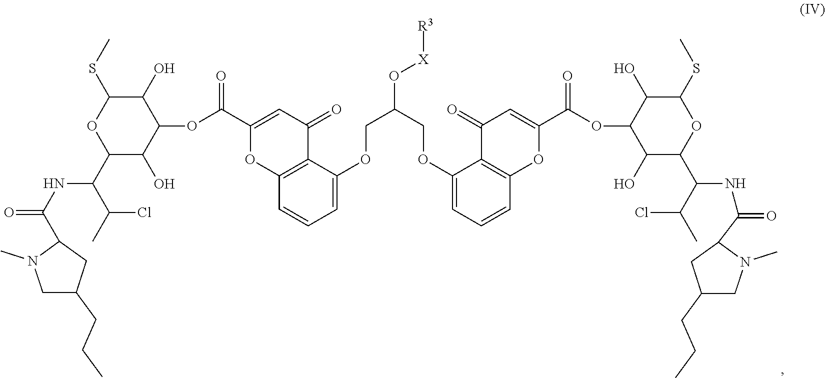
or a salt thereof, wherein:
X is —C(O)— or is a bond; and
R³ is hydrogen or is chosen from alkyl, cycloalkyl, heterocycloalkyl, alkenyl, alkoxy, aryl, heteroaryl, 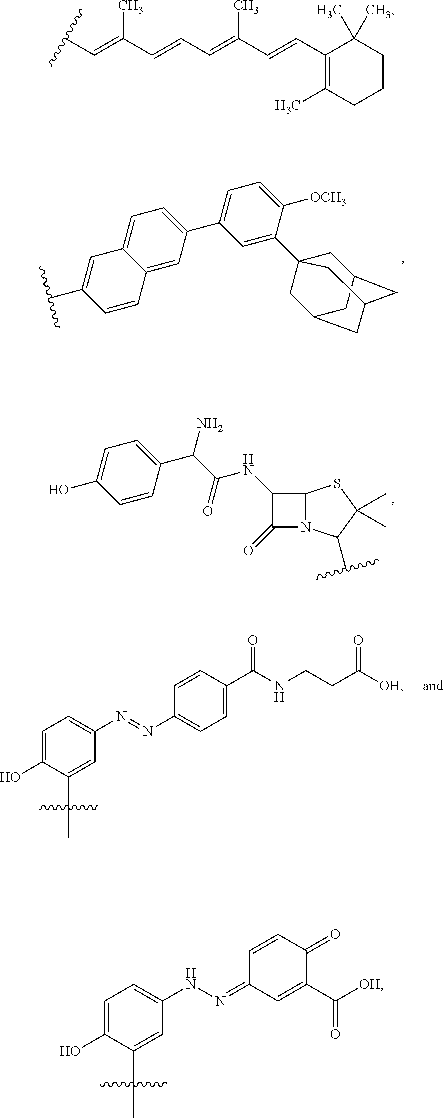 [SMILES: */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *c1cc(/N=N/c2ccc(C(=O)NCCC(=O)O)cc2)ccc1O ; *c1cc(N/N=C2\C=CC(=O)C(C(=O)O)=C2)ccc1O]
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

16. The compound of claim 1, wherein the compound is a compound of Formula V: 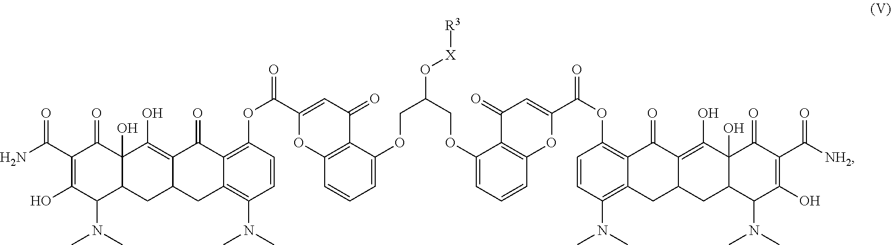
or a salt thereof, wherein:
X is —C(O)— or is a bond; and
R³ is hydrogen or is chosen from alkyl, cycloalkyl, heterocycloalkyl, alkenyl, alkoxy, aryl, heteroaryl, 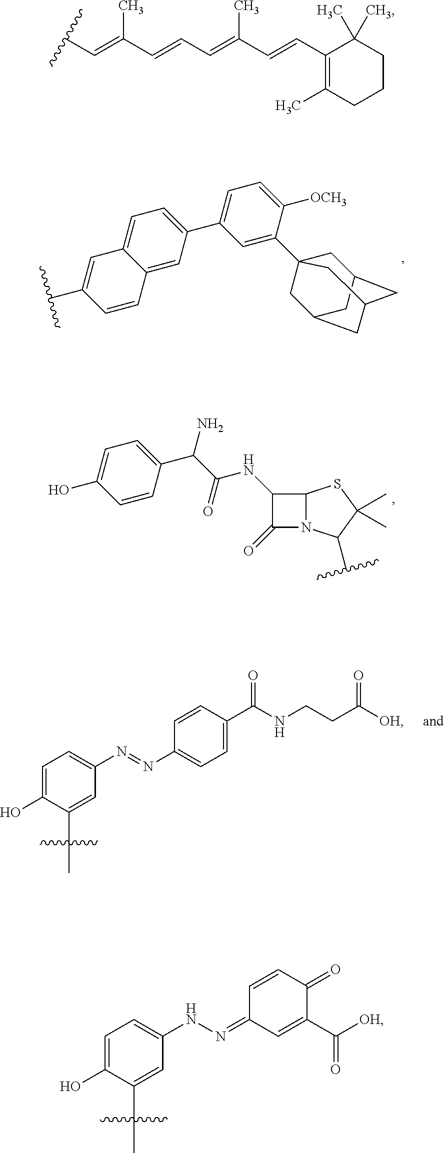 [SMILES: */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *c1cc(/N=N/c2ccc(C(=O)NCCC(=O)O)cc2)ccc1O ; *c1cc(N/N=C2\C=CC(=O)C(C(=O)O)=C2)ccc1O]
any of which may be optionally substituted by one or more R⁴,
each R⁴ being independently chosen from alkyl, cycloalkyl, heterocycloalkyl, alkenyl, alkynyl, aryl, heteroaryl, amino, amido, hydroxyl, alkoxy, halo, haloalkyl, haloalkoxy, and carboxyl.

17-25. (canceled)

26. The compound of claim 1, wherein the compound is a compound of Formula VI: 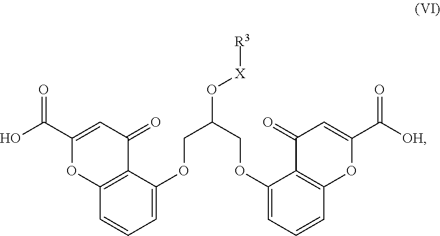
or a salt thereof, wherein:
R¹ is chosen from

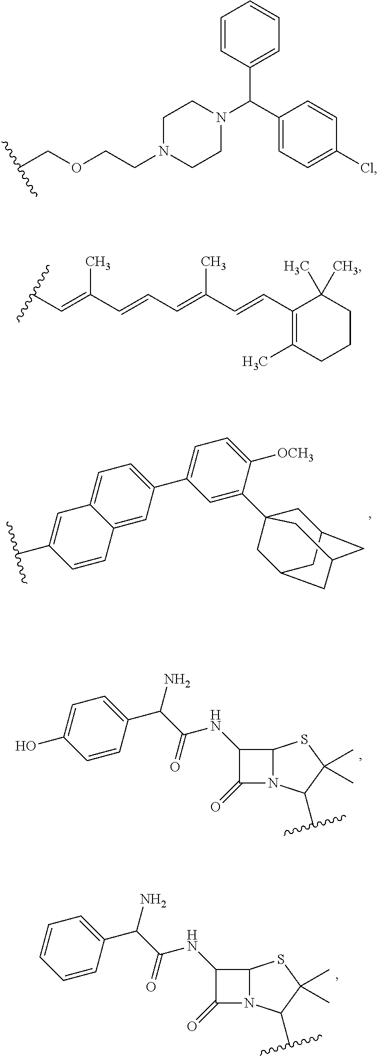 [SMILES: *COCCN1CCN(C(c2ccccc2)c2ccc(Cl)cc2)CC1 ; */C=C(C)/C=C/C=C(C)/C=C/C1=C(C)CCCC1(C)C ; *c1ccc2cc(-c3ccc(OC)c(C45CC6CC(CC(C6)C4)C5)c3)ccc2c1 ; *C1N2C(=O)C(NC(=O)C(N)c3ccc(O)cc3)C2SC1(C)C ; *C1N2C(=O)C(NC(=O)C(N)c3ccccc3)C2SC1(C)C]

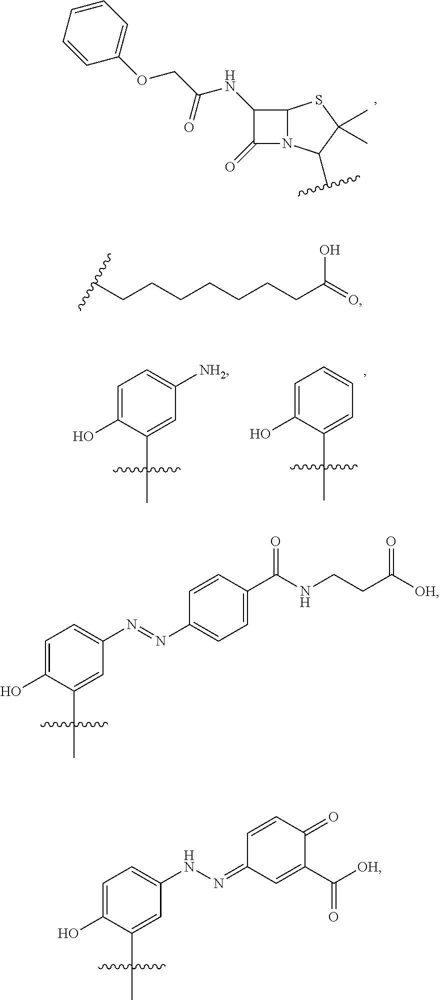 [SMILES: *C1N2C(=O)C(NC(=O)COc3ccccc3)C2SC1(C)C ; *CCCCCCCC(=O)O ; *c1cc(N)ccc1O ; *c1ccccc1O ; *c1cc(/N=N/c2ccc(C(=O)NCCC(=O)O)cc2)ccc1O ; *c1cc(N/N=C2\C=CC(=O)C(C(=O)O)=C2)ccc1O]

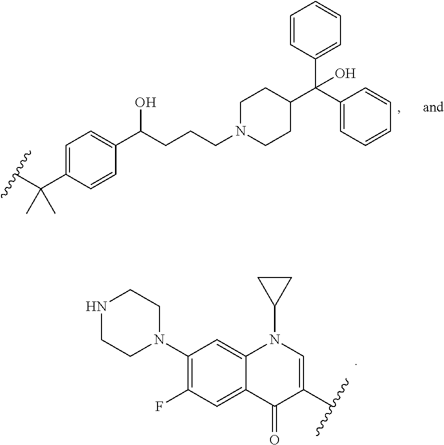 [SMILES: *C(C)(C)c1ccc(C(O)CCCN2CCC(C(O)(c3ccccc3)c3ccccc3)CC2)cc1 ; *c1cn(C2CC2)c2cc(N3CCNCC3)c(F)cc2c1=O]

27. The compound of claim 1, wherein the compound is chosen from 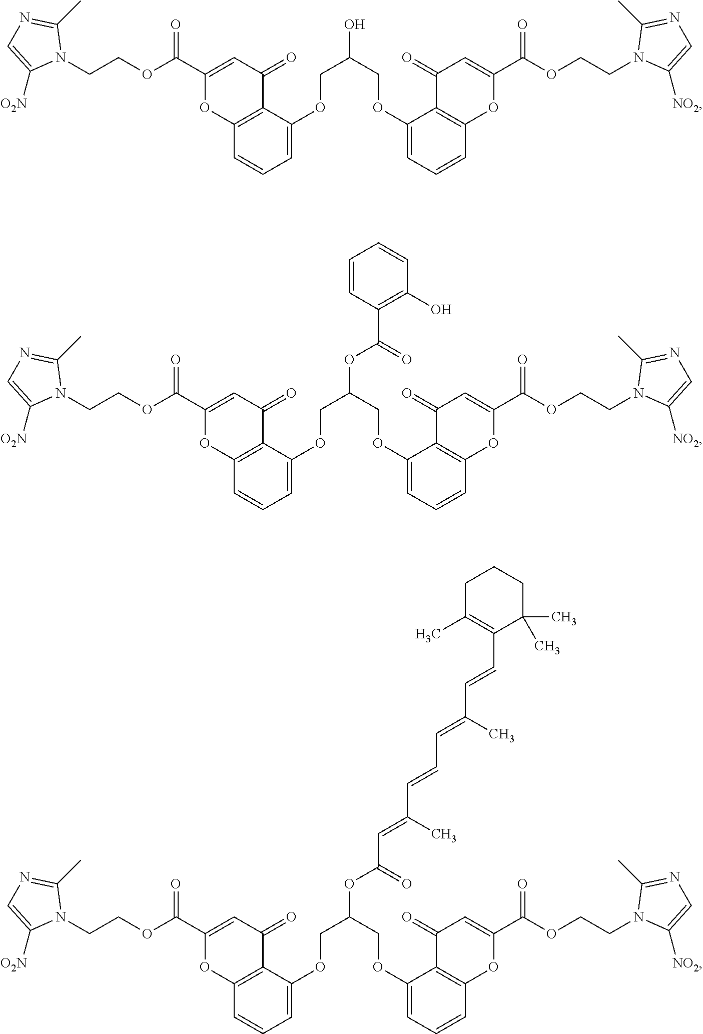 [SMILES: Cc1ncc([N+](=O)[O-])n1CCOC(=O)c1cc(=O)c2c(OCC(O)COc3cccc4oc(C(=O)OCCn5c([N+](=O)[O-])cnc5C)cc(=O)c34)cccc2o1 ; Cc1ncc([N+](=O)[O-])n1CCOC(=O)c1cc(=O)c2c(OCC(COc3cccc4oc(C(=O)OCCn5c([N+](=O)[O-])cnc5C)cc(=O)c34)OC(=O)c3ccccc3O)cccc2o1 ; CC1=C(/C=C/C(C)=C/C=C/C(C)=C/C(=O)OC(COc2cccc3oc(C(=O)OCCn4c([N+](=O)[O-])cnc4C)cc(=O)c23)COc2cccc3oc(C(=O)OCCn4c([N+](=O)[O-])cnc4C)cc(=O)c23)C(C)(C)CCC1] 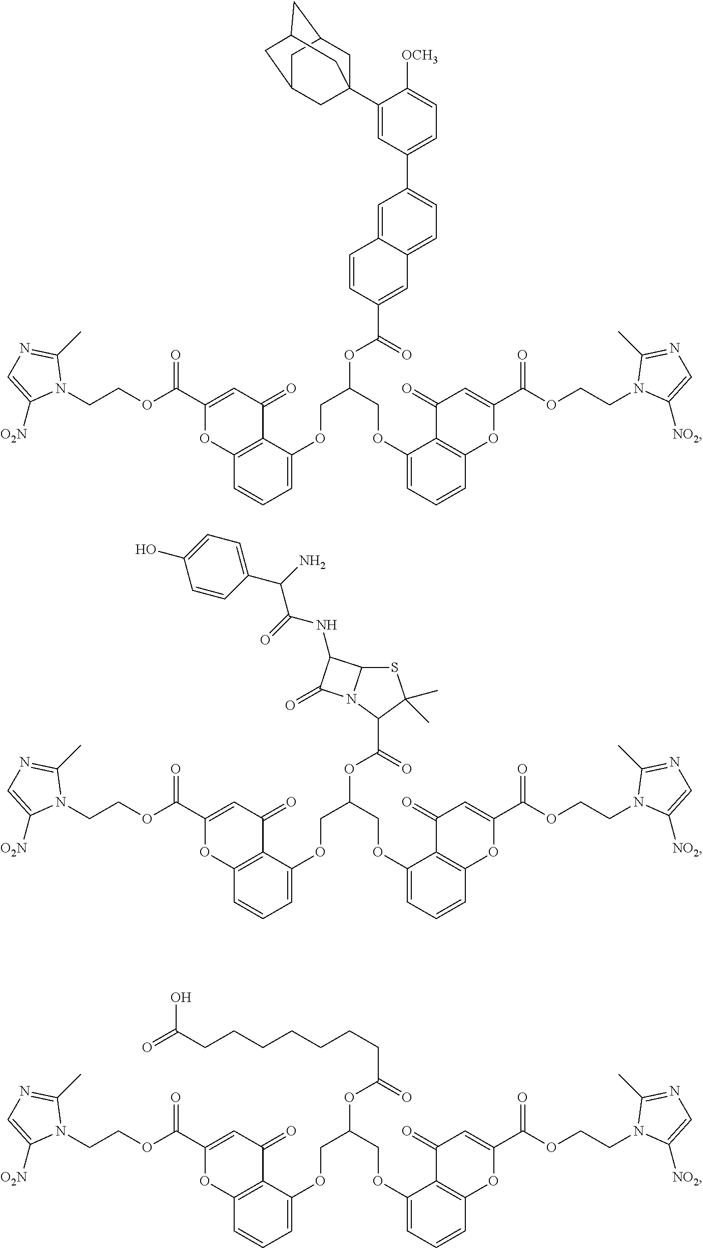 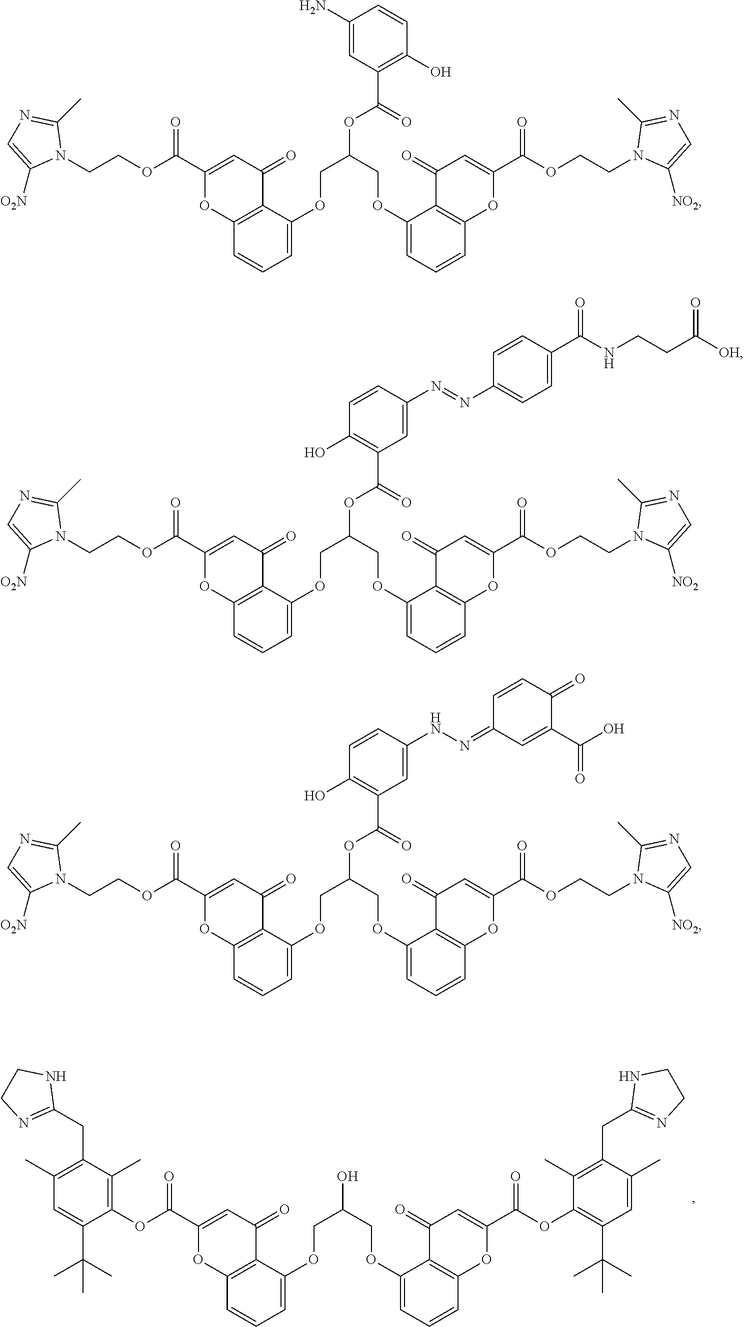 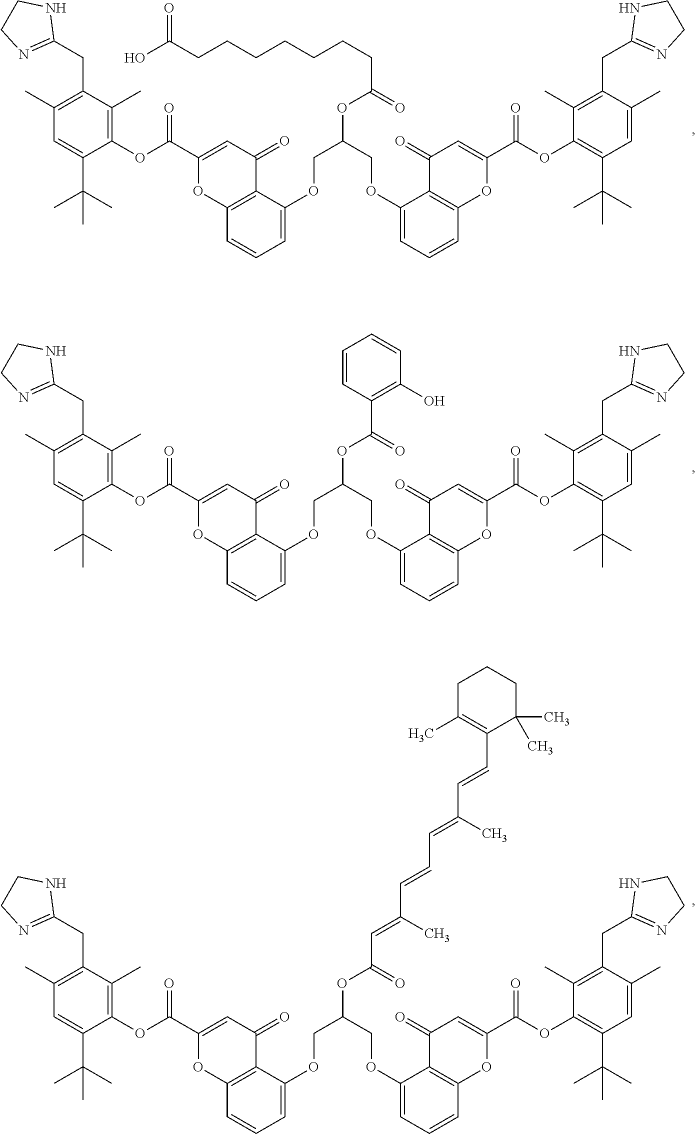 [SMILES: Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)CCCCCCCC(=O)O)cccc3o2)c(C)c1CC1=NCCN1 ; Cc1cc(C(C)(C)C)c(OC(=O)c2cc(=O)c3c(OCC(COc4cccc5oc(C(=O)Oc6c(C(C)(C)C)cc(C)c(CC7=NCCN7)c6C)cc(=O)c45)OC(=O)c4ccccc4O)cccc3o2)c(C)c1CC1=NCCN1 ; CC1=C(/C=C/C(C)=C/C=C/C(C)=C/C(=O)OC(COc2cccc3oc(C(=O)Oc4c(C(C)(C)C)cc(C)c(CC5=NCCN5)c4C)cc(=O)c23)COc2cccc3oc(C(=O)Oc4c(C(C)(C)C)cc(C)c(CC5=NCCN5)c4C)cc(=O)c23)C(C)(C)CCC1] 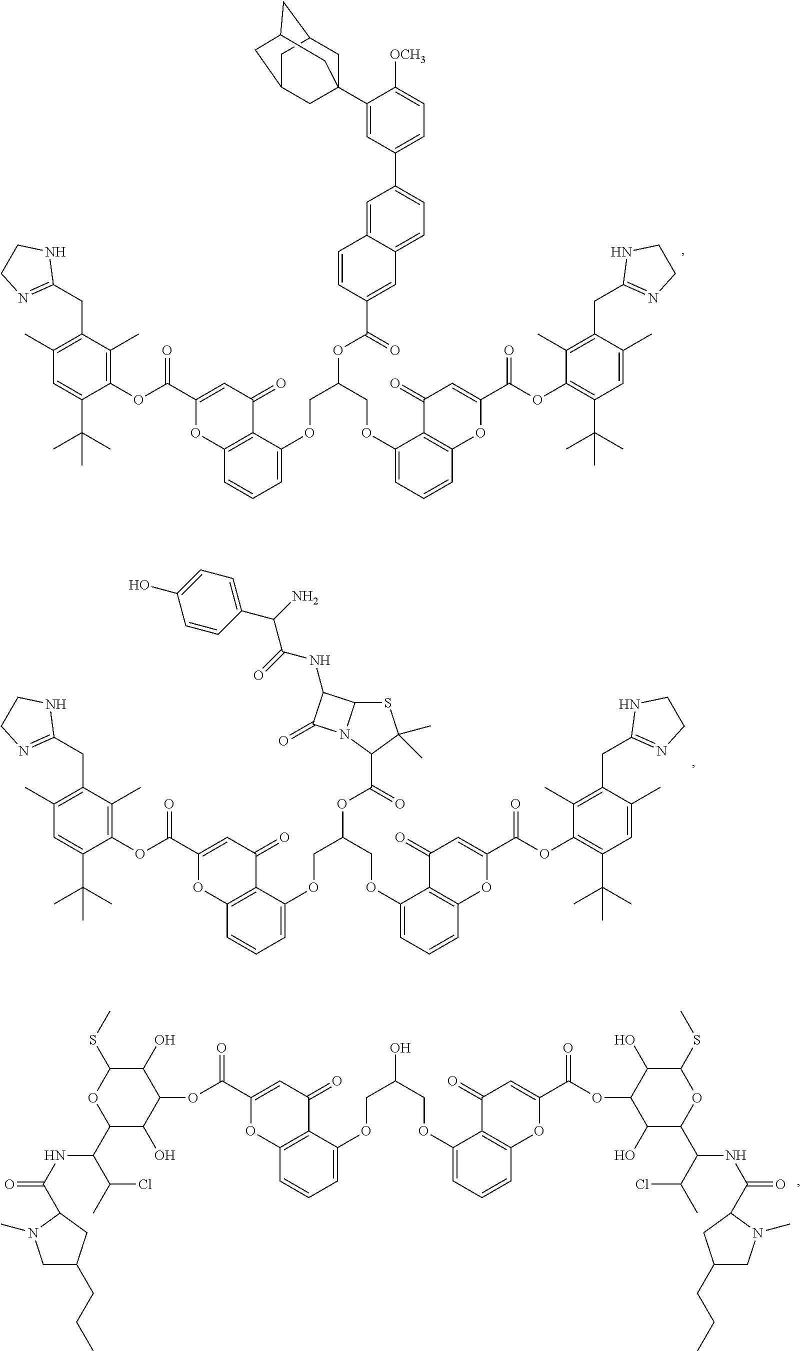 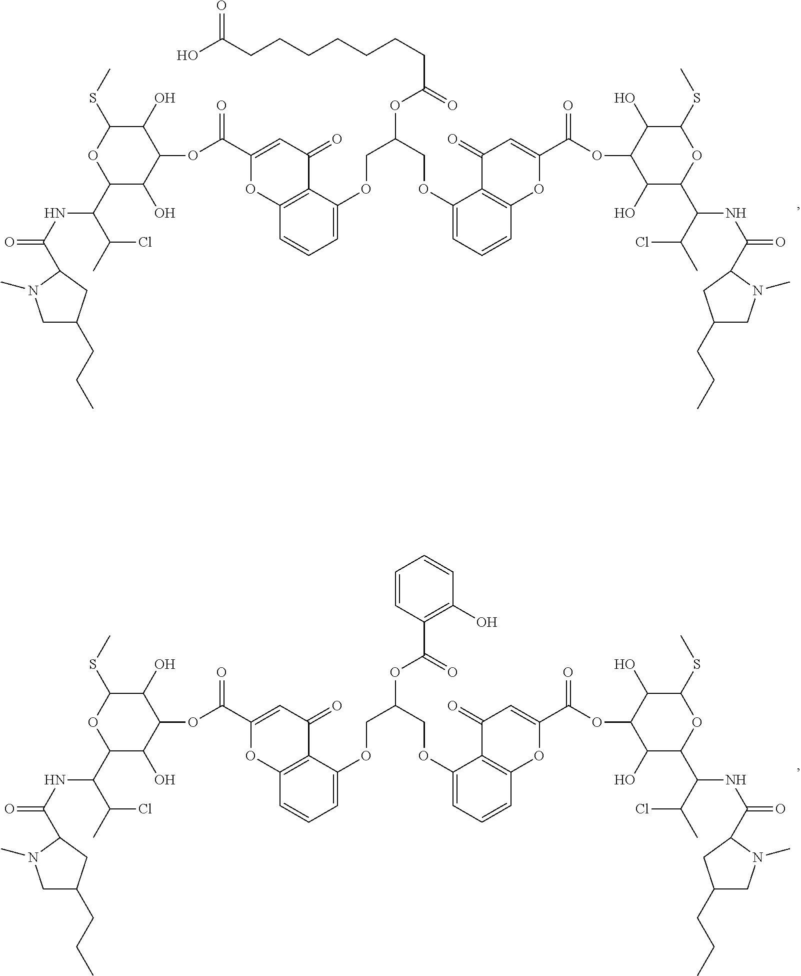 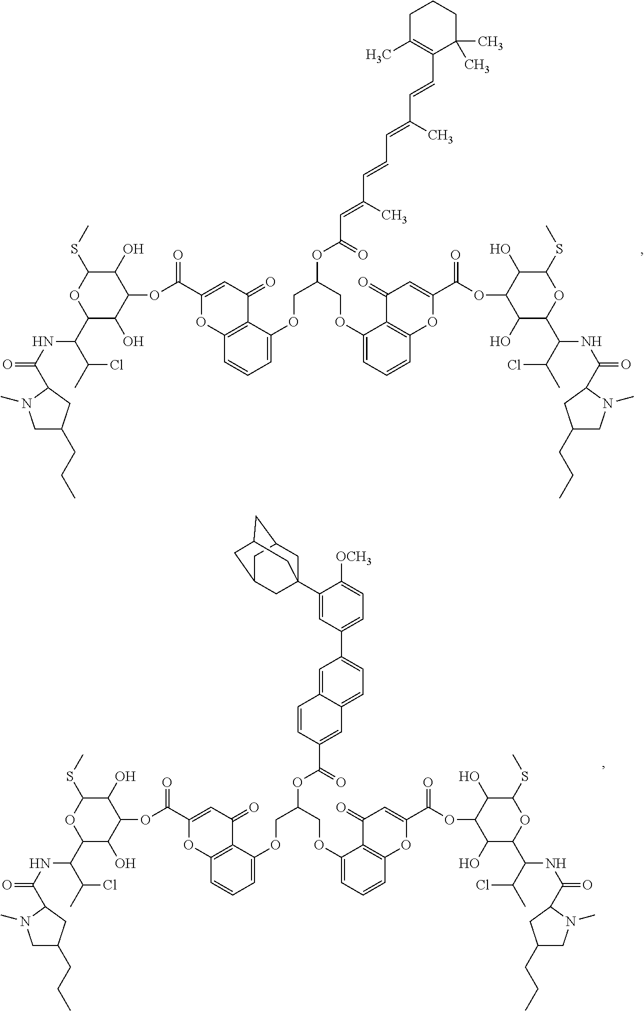 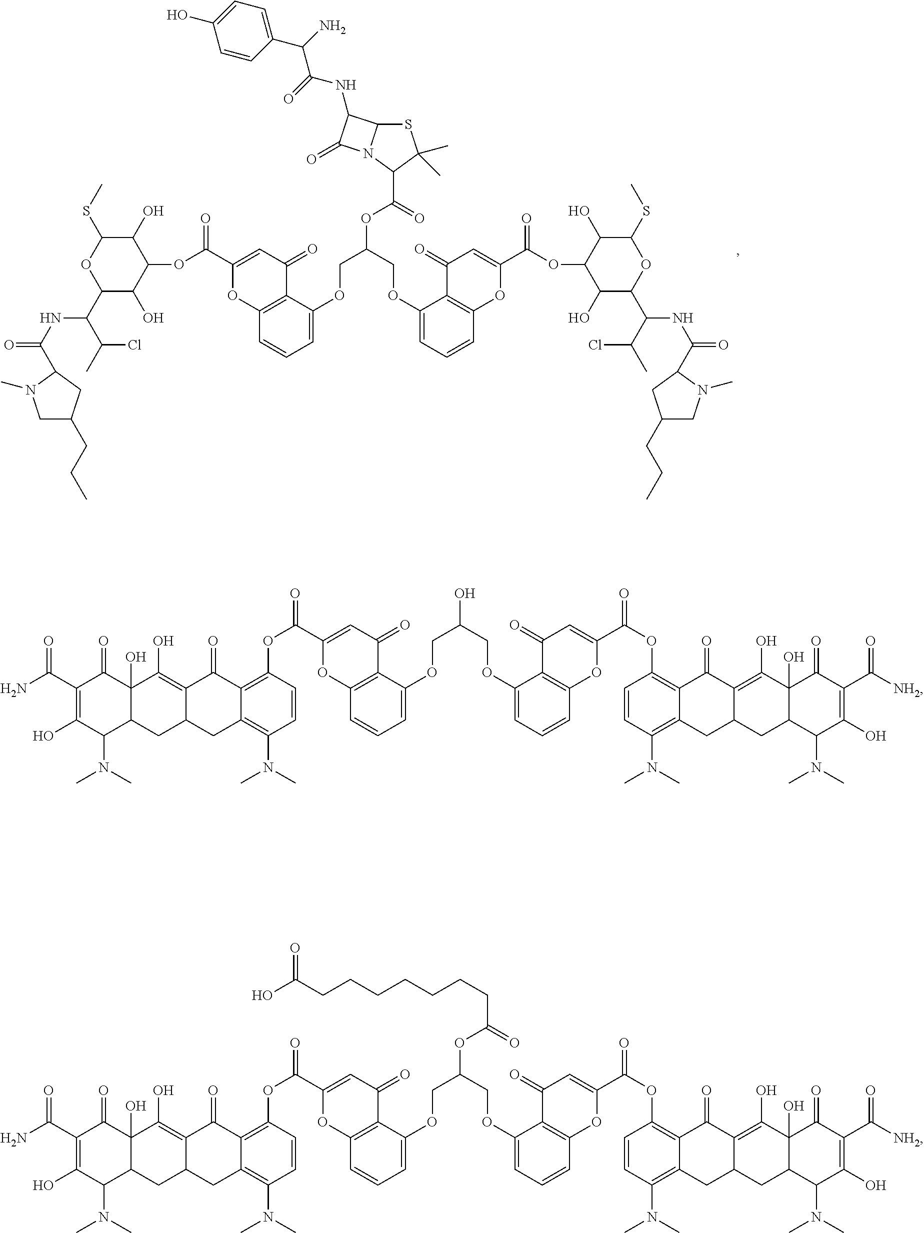 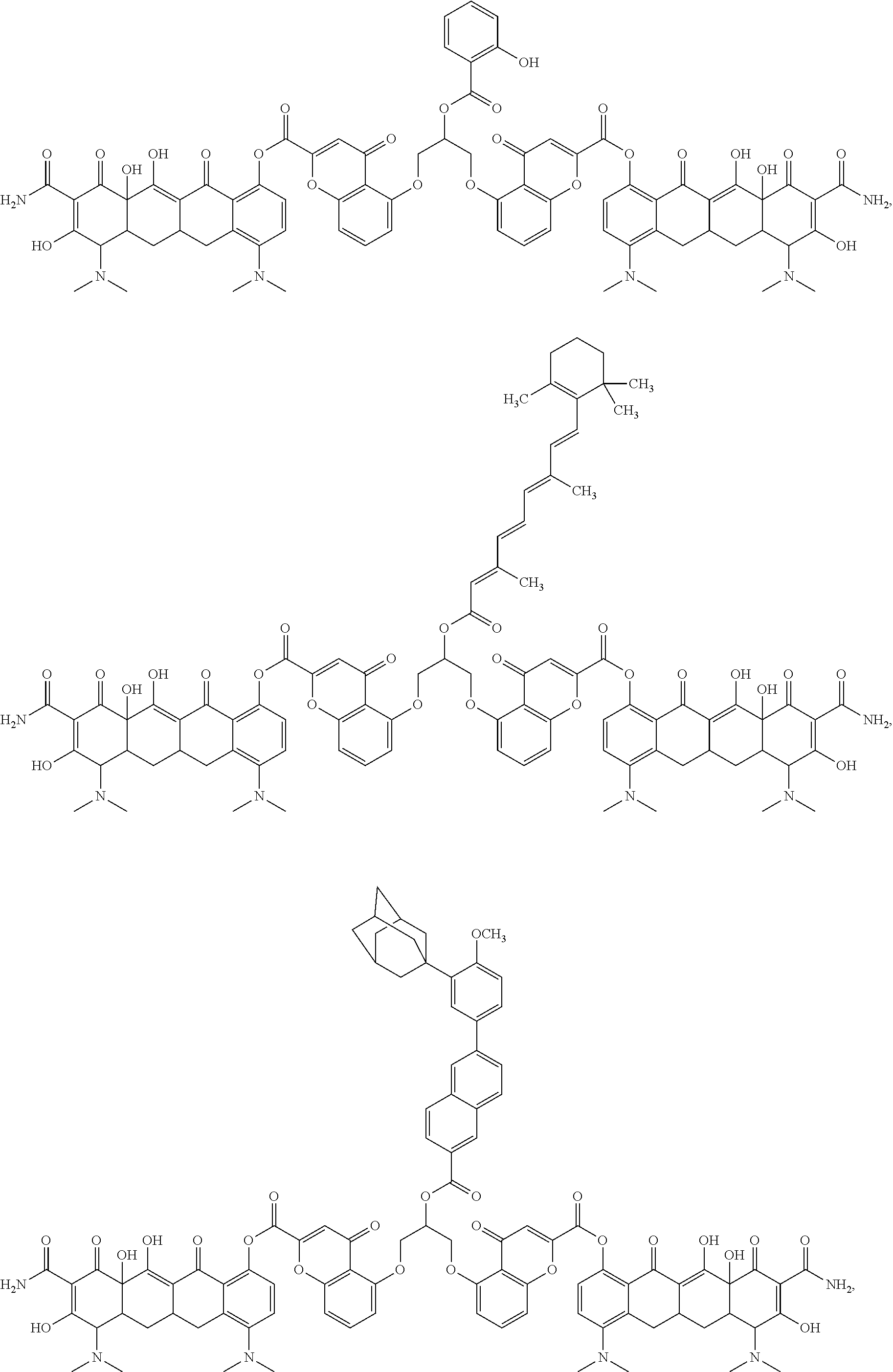 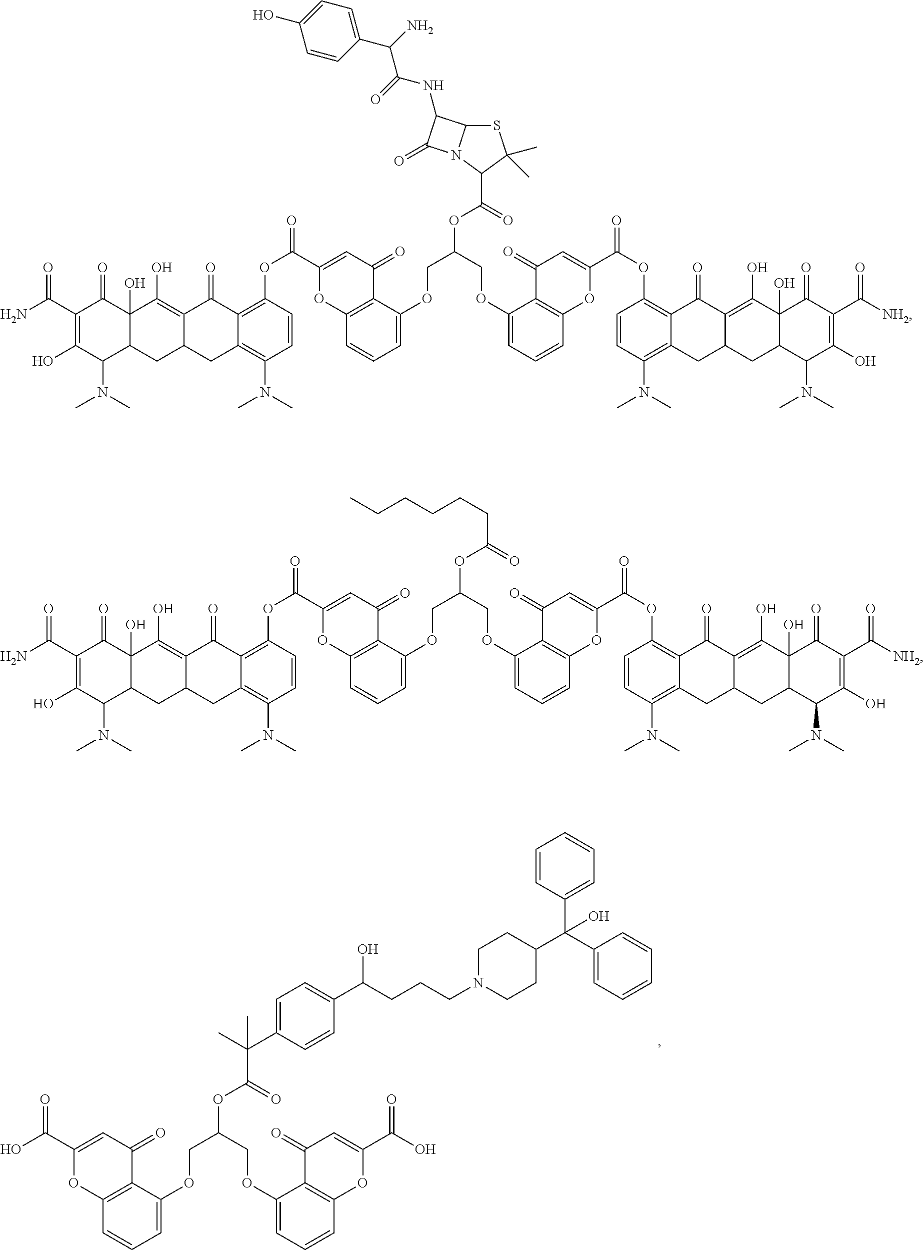 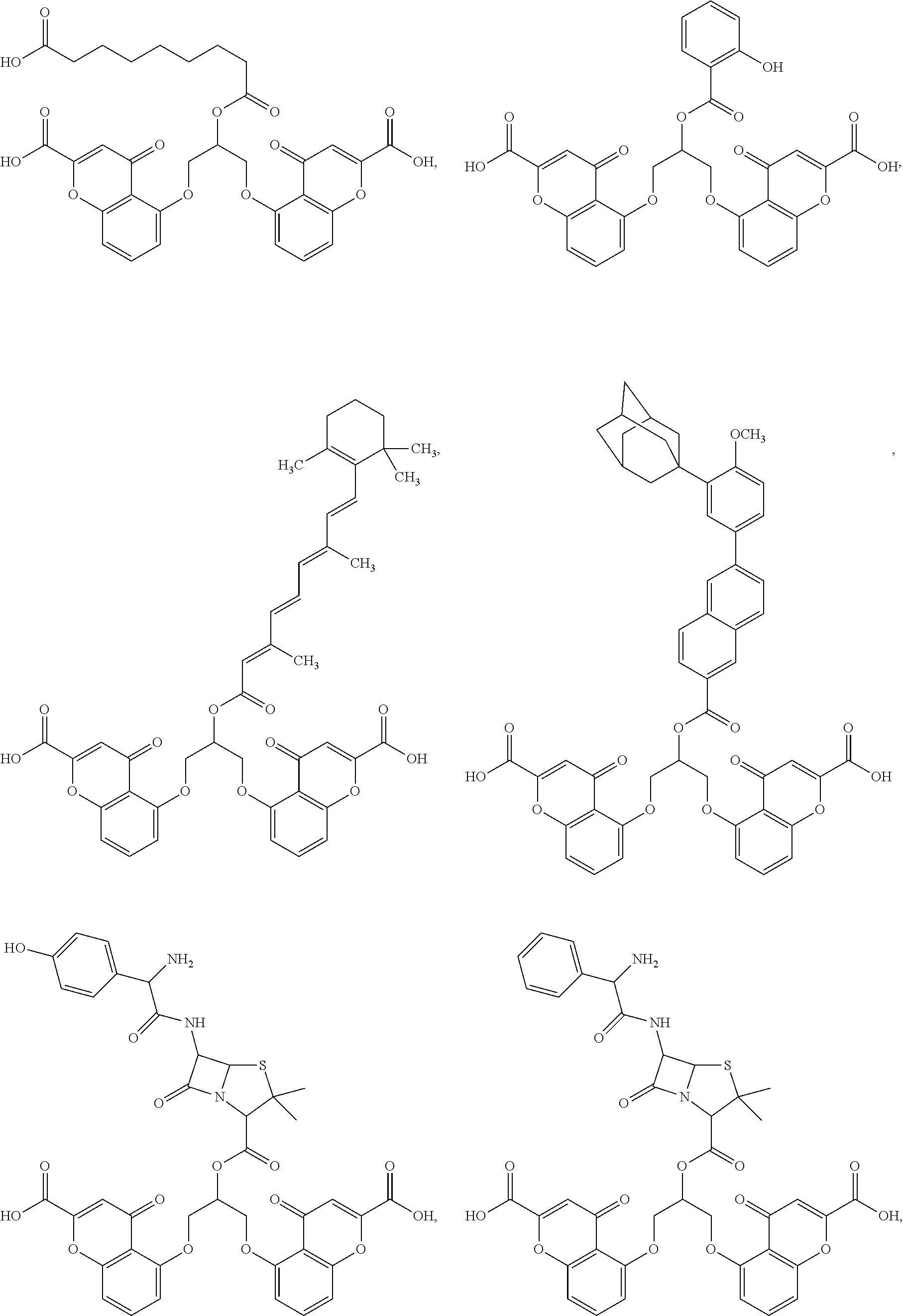 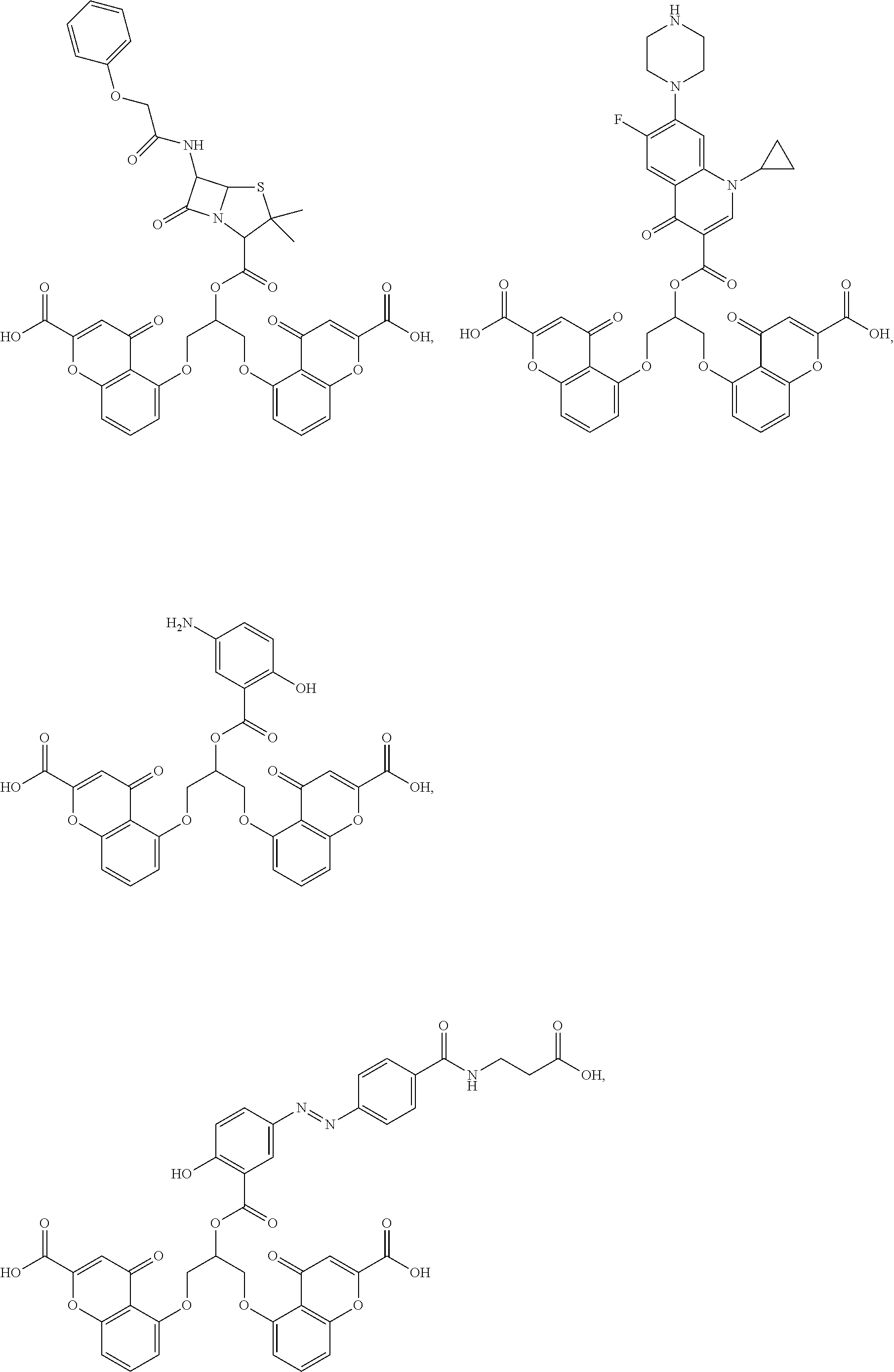 [SMILES: CC1(C)SC2C(NC(=O)COc3ccccc3)C(=O)N2C1C(=O)OC(COc1cccc2oc(C(=O)O)cc(=O)c12)COc1cccc2oc(C(=O)O)cc(=O)c12 ; O=C(O)c1cc(=O)c2c(OCC(COc3cccc4oc(C(=O)O)cc(=O)c34)OC(=O)c3cn(C4CC4)c4cc(N5CCNCC5)c(F)cc4c3=O)cccc2o1 ; Nc1ccc(O)c(C(=O)OC(COc2cccc3oc(C(=O)O)cc(=O)c23)COc2cccc3oc(C(=O)O)cc(=O)c23)c1 ; O=C(O)CCNC(=O)c1ccc(/N=N/c2ccc(O)c(C(=O)OC(COc3cccc4oc(C(=O)O)cc(=O)c34)COc3cccc4oc(C(=O)O)cc(=O)c34)c2)cc1] 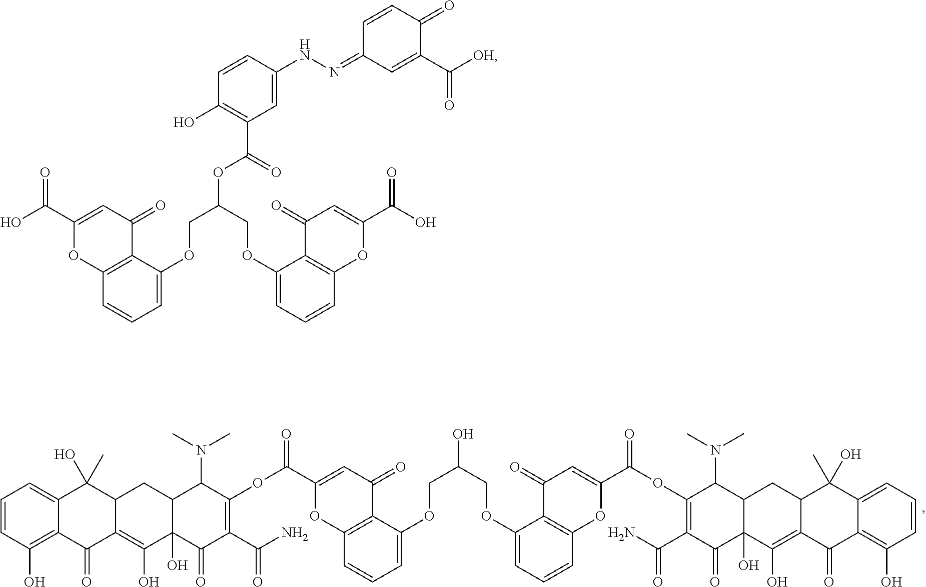 [SMILES: O=C(O)C1=C/C(=N/Nc2ccc(O)c(C(=O)OC(COc3cccc4oc(C(=O)O)cc(=O)c34)COc3cccc4oc(C(=O)O)cc(=O)c34)c2)C=CC1=O ; CN(C)C1C(OC(=O)c2cc(=O)c3c(OCC(O)COc4cccc5oc(C(=O)OC6=C(C(N)=O)C(=O)C7(O)C(O)=C8C(=O)c9c(O)cccc9C(C)(O)C8CC7C6N(C)C)cc(=O)c45)cccc3o2)=C(C(N)=O)C(=O)C2(O)C(O)=C3C(=O)c4c(O)cccc4C(C)(O)C3CC12] 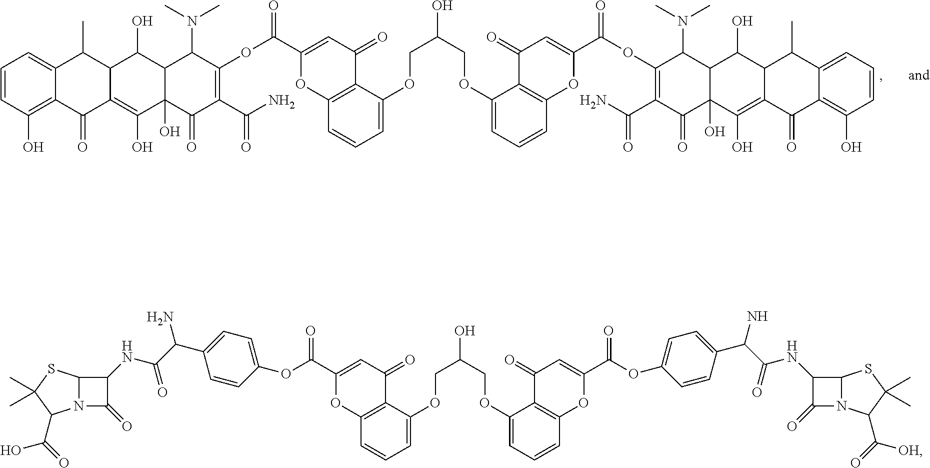
or a salt thereof.

28. A pharmaceutical formulation comprising a compound of claim 1, or a salt thereof, together with a pharmaceutically acceptable carrier.

29-36. (canceled)

37. A method of treatment of a skin condition, comprising administering a therapeutically effective amount of a compound of claim 1, or a salt thereof, to a patient in need thereof.

38. The method of claim 37, wherein the skin condition is characterized by elevated levels of mast cells, basophils, eosinophils, histamine, bradykinin, leukotriene C₄, prostaglandin D2, or a combination thereof.

39. The method of claim 38, wherein the skin condition is characterized by elevated levels of mast cells.

40. The method of claim 39, wherein the skin condition is urticaria.

41. (canceled)

42. The method of claim 37, wherein the skin condition is chosen from atopic dermatitis, rosacea, prurigo nodularis, and acne vulgaris.

43. The method of claim 37, wherein the administration is topical.

44. (canceled)
